(19) United States
(12) Patent Application Publication
(10) Pub. No.: US 2024/0279328 A1
(43) Pub. Date: Aug. 22, 2024

(54) METHODS TO GLYCOENGINEER PROTEINS

(71) Applicant: THE JOHNS HOPKINS UNIVERSITY, Baltimore, MD (US)

(72) Inventors: Kevin J. Yarema, Woodstock, MD (US); Christian Agatemor, Baltimore, MD (US); Christopher T. Saeui, Rockville, MD (US); Matthew Buettner, Baltimore, MD (US); Jamie SPANGLER, Baltimore, MD (US); Seth Ludwig, Baltimore, MD (US); Riddhima Dennar Chitre, Baltimore, MD (US); Kris Dammem-Brower, Baltimore, MD (US); Zachary BERNSTEIN, Baltimore, MD (US)

(21) Appl. No.: 18/013,864

(22) PCT Filed: Jul. 2, 2021
(86) PCT No.: PCT/US21/40262

§ 371 Date: Dec. 29, 2022

Related U.S. Application Data
(60) Provisional application No. 63/047,358, filed on Jul. 2, 2020.

(51) Int. Cl. C07K 16/24; A61K 39/00; A61P 35/00; C12N 9/10; C12N 9/24; C12N 9/88; C12N 9/90; C12N 15/52

(52) U.S. Cl. CPC C07K 16/246; A61P 35/00; C12N 9/1081; C12N 9/1085; C12N 9/2402; C12N 9/88; C12N 9/90; C12N 15/52; A61K 2039/505; C07K 2317/14; C07K 2317/41; C12Y 204/01; C12Y 205/01057; C12Y 302/01018; C12Y 401/03003; C12Y 501/03008

(57) ABSTRACT

Compositions for producing glycoengineered proteins, e.g. antibodies, include host cells which lack the ability to produce enzymes that modulate sialic acid metabolic flux.

### CROSS-REFERENCE TO RELATED APPLICATIONS

This application is a 35 U.S.C. § 371 national phase entry of International Patent Application No. PCT/US2021/040262 filed Jul. 2, 2021, which claims priority to U.S. Provisional Application No. 63/047,358 filed Jul. 2, 2020, the disclosures of which are incorporated herein by reference in their entirety.

### SEQUENCE LISTING

This application contains a Sequence Listing which has been submitted electronically in ASCII format and is hereby incorporated by reference in its entirety. Said ASCII copy, created on Aug. 23, 2021, is named Sequence_Listing_348358-07801.txt, and is 49,887 bytes in size.

### FIELD OF THE DISCLOSURE

This disclosure relates proteins and immunoglobulins modified with N-glycans groups. Uses include conjugation of functional and non-functional groups with the N-glycans, providing therapeutic proteins with new bioactivities, and improving their pharmacological properties.

### BACKGROUND

Chinese hamster ovary (CHO) cells are the “workhorse” cell line for biomanufacturing, currently accounting for ˜90% of therapeutic antibody production (and similar levels for other recently-developed therapeutic proteins). Despite their widespread usage, CHO cells do not provide optimal glycosylation, which is now widely recognized to be critical for the safety, bioactivity, pharmacological properties, and overall efficacy of protein-based therapeutics.

### SUMMARY

The disclosure provides for glycoengineered proteins, host cells for producing the glycoengineered proteins, and methods of producing these glycoengineered proteins.

We also now provide an integrated platform that combines several distinct aspects of glycoengineering to optimize the glycosylation patterns of existing proteins in order to create superior molecular therapeutics.

In a first aspect, an improved technology platform is provided herein for the biomanufacturing of improved therapeutic proteins with better safety, pharmacokinetic, and pharmacodynamic properties that can be achieved together with new therapeutic efficacy and activity modalities. These objectives are achieved by combining three complementary “glycoengineering” approaches. Chinese hamster ovary (CHO) cells are the “workhorse” cell line for biomanufacturing, currently accounting for ˜90% of therapeutic antibody production (and similar levels for other recently-developed therapeutic proteins). Despite their widespread usage, CHO cells do not provide optimal glycosylation, which is now widely recognized to be critical for the safety, bioactivity, pharmacological properties, and overall efficacy of protein-based therapeutics. This disclosure describes genetic modification to the host production cells that builds in the expression of key proteins involved in glycoprotein production that are lacking in wild-type CHO cells OR knocks out other key genes that interfere with optimal glycosylation.

In a second aspect, the genetic modification of the host cells was designed to be closely complementary with hexosamine analogs used to supplement the production of engineered proteins. For example, wild-type CHO cells lack α2,6-sialyltransferase and thus cannot produce therapeutic proteins with α2,6-sialylation, which is important for the pharmacokinetic/pharmacodynamic PK/PD behavior of proteins in humans. It was found that expression of α2,6-sialyltransferase alone does not significantly improve sialylation but co-supplementation with a ManNAc analog designed to increase flux through the sialic acid biosynthetic pathway resulting in synergistic improvements. In another iteration of this technology, non-natural monosaccharide analogs can be used in the genetically modified host cells to introduce chemical functional groups into the engineered proteins. These chemical functional groups can be used via bioorthogonal ligation reactions to derivatize the proteins with additional chemical moieties—e.g., in the context of antibody drug conjugates with small molecule therapeutics—in ways that overcome limitations of current conjugation methods.

In a third aspect, the platform comprises genetic modification of the engineered protein by introducing consensus sequons that add (or delete) an N-glycan attachment site. By using this method, two or more glycans can be added to a protein (e.g., a therapeutic antibody) without affecting the protein's normal biological activity. In some iterations, this approach (when combined with the use of genetically modified host cells and the appropriate monosaccharide analog) can dramatically improve PK/PD. In a second iteration, addition of N-glycans can increase the number of sites for conjugation; for example, two additional N-glycans per IgG chain increases the potential drug to antibody ratio from 4:1 to 22:1. These objectives can be achieved by the combined use of this three-pronged platform.

Accordingly, in certain embodiments, a method of producing a modified protein or peptide comprises transfecting a host cell with an expression vector encoding an protein or peptide comprising one or more genetic mutations, wherein the one or more genetic mutations encode for one or more N-glycans; thereby, producing a modified or peptide.

In these and other embodiments, the host cell does not produce or encode one or more enzymes that modulate sialic acid metabolic flux. In certain embodiments, the method further comprises contacting the host cell with one or more hexosamine analogs. For example, the levels of non-natural analog incorporation into the protein or peptide is enhanced by the knock-out of the one or more enzymes that modulate sialic acid metabolic flux such as, for example, GNE (UDP-N-acetylglucosamine 2-epimerase/N-acetylmannosamine kinase), RENBP (Renin-binding protein; GlcNAc 2-epimerase) in the host cell. GNE and RENBP are the two enzymes that naturally regulate flux into the sialic acid biosynthetic pathway.

In certain embodiments a modified host cell comprises one or more nucleic acid sequences encoding one or more enzymes that modulate N-glycan branching, sialylation or a combination thereof. In certain embodiments, the host cell is transformed with one or more vectors comprising one or more nucleic acid sequences encoding for one or more enzymes are expressed by a vector enzymes that modulate N-glycan branching, sialylation or combinations thereof. The one or more enzymes that modulate N-glycan branching comprise N-acetylglucosaminyltransferases. The one or more enzymes that modulate sialylation comprise sialyltransferases.

In these and other embodiments, the host cell genome is manipulated so that the host cell does not produce or encode one or more enzymes that modulate sialic acid metabolic flux or recycling/degradation of sialylated proteins. In certain embodiments the nucleic acid sequences encoding for one or more enzymes that modulate sialic acid metabolic flux are inactivated or excised from the host cell's genome. For example, the host cells are contacted with a gene-editing agent which inactivates regulatory or other components that encode for these enzymes or by using a multiplex approach whereby the gene editing agent is targeted at both ends of the nucleic acid segment to be excised. In certain embodiments, the one or more enzymes that modulate sialic acid metabolic flux comprise GNE (UDP-N-acetylglucosamine 2-epimerase/N-acetylmannosamine kinase), RENBP (Renin-binding protein; N-GlcNAc 2-epimerase), NPL (N-acetylneuraminic acid lyase), NEU1 (Neurimindase 1), NEU2 (Neurimindase 2), NEU3 (Neurimindase 3), NEU4 (Neurimindase 4) or combinations thereof.

In certain embodiments, the host cell lacking the one or more enzymes that modulate sialic acid metabolic flux is transformed with one or more vectors encoding for one or more N-acetylglucosaminyltransferases, sialyltransferases or a combination thereof. For example, a GNE knock-out host cell also over expresses human sialyltransferase (e.g., ST6GAL1) and branching enzymes. Accordingly, once the GNE enzyme is knocked out of host cells, endogenous flux of ManNAc into the sialic acid biosynthetic pathway is eliminated. As a result, flux into the sialic acid biosynthetic pathway is dominated by the supplementation of the culture medium with sialic acid precursors.

In certain embodiments, the host cells are transformed to encode for selective substrate preferences of certain sialyltransferases. Accordingly, these host cells selectively install natural or non-natural sialic acids on particular glycoproteins, and at specific sites on a glycoprotein.

In these and other embodiments, the host cell comprises: primary cells, BSC cells, HeLa cells, HepG2 cells, LLC-MK cells, CV-1 cells, COS cells, VEROc ells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, CHO cells, NS-1 cells, MRC-5 cells, WI-38 cells, BHK cells, 3T3 cells, 293 cells, RK cells, Per.C6 cells or chicken embryo cells. In certain embodiments, the host cells are CHO cells, HEK293 cells, or BHK cells. In certain embodiments, the CHO host cells are CHO-K1 cells.

In certain embodiments, an engineered protein comprises one or more modified amino acid sequences, wherein the modified amino acid sequences comprise one or more N-glycan consensus sequences. In certain embodiments, the N-glycan consensus sequence comprises Asn-X-Ser/Thr, where X is any amino acid except proline (Pro). In certain embodiments, the engineered protein comprises one or more hexosamine analogs. In certain embodiments, the engineered protein further comprises one or more functional groups and/or non-functional groups. In certain embodiments, a functional group comprises: an azido group, thiol group, alkyne, alkyl, alkenyl, alkynyl, carboxamido, aldehydes or combinations thereof.

In these and other embodiments, the engineered protein is an immunoglobulin or another protein. In certain embodiments, the protein comprises: cytokines, immunogenic peptides, viral immunogenic peptides, interferons, immune regulatory proteins, mimetics, hormones, enzymes, receptor or combinations thereof. In certain embodiments, the immunoglobulin or therapeutic protein comprises one or more labels, compounds or combinations thereof.

The engineered proteins can be conjugated to a group or agent, for example, antibody-drug conjugates. In these and other embodiments, the one or more agents are conjugated to the functional groups. In certain embodiments, the one or more agents comprise therapeutic agents, toxins, labels or combinations thereof.

In certain embodiments, a modified immunoglobulin comprises one or more N-glycan groups. In certain embodiments, the immunoglobulin comprises at least one amino acid sequence comprising SEQ ID NO: 1-12.

In certain embodiments, a method of treating cancer comprising administering to a subject in need thereof, a pharmaceutical composition comprising a therapeutically effective amount of the engineered protein embodied herein.

In certain embodiments, a method of treating an autoimmune disease, comprising administering to a subject in need thereof, a pharmaceutical composition comprising a therapeutically effective amount of the engineered protein embodied herein.

In certain embodiments, a method of treating an inflammatory disease comprising administering to a subject in need thereof, a pharmaceutical composition comprising a therapeutically effective amount of the engineered protein embodied herein.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

Antibody “effector functions” refer to those biological activities attributable to the Fc region (a native sequence Fc region or amino acid sequence variant Fc region) of an antibody. Examples of antibody effector functions include Clq binding, complement dependent cytotoxicity (CDC), Fc receptor binding, antibody-dependent cell-mediated cytotoxicity (ADCC), etc. In one embodiment, the antibody herein essentially lacks effector function.

The term “antibody-dependent cellular cytotoxicity (ADCC)” refers to lysis of human target cells by an antibody in the presence of effector cells. The term “complement-dependent cytotoxicity (CDC)” denotes a process initiated by binding of complement factor Clq to the Fc part of most IgG antibody subclasses. Binding of Clq to an antibody is caused by defined protein-protein interactions at the so called binding site. Such Fc part binding sites are known in the state of the art. Such Fc part binding sites are, e.g., characterized by the amino acids L234, L235, D270, N297, E318, K320, K322, P331, and P329 (numbering according to EU index of Kabat). Antibodies of subclass IgG1, IgG2, and IgG3 usually show complement activation including Clq and C3 binding, whereas IgG4 does not activate the complement system and does not bind Clq and/or C3.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the terms “fucose,” “core fucose,” and “core fucose residue” are used interchangeably and refer to a fucose in α-1,6-position linked to the N-acetylglucosamine.

As used herein, the term “glycan” refers to a polysaccharide, oligosaccharide or monosaccharide. Glycans can be monomers or polymers of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, the terms “N-glycan”, “N-linked glycan”, “N-linked glycosylation”, “Fc glycan” and “Fc glycosylation” are used interchangeably and refer to an N-linked oligosaccharide attached by an N-acetylglucosamine (GlcNAc) linked to the amide nitrogen of an asparagine residue in a Fc-containing polypeptide. The term “Fc-containing polypeptide” refers to a polypeptide, such as an antibody, which comprises an Fc region.

As used herein, the term “glycoengineered” when used herein refers to N-glycan on a protein or antibody has been altered or engineered either enzymatically or chemically. The term “glycoengineering” as used herein refers to the methods embodied herein to produce the glycoengineered Fc.

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloic-derived phagocytes. “Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

To “treat” a disease as the term is used herein, means to reduce the frequency or severity of at least one sign or symptom of a disease or disorder experienced by a subject.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic representation illustrating glycoengineering of the doubly-modified 602-M18 variant of the 602 antibody with emphasis on the four newly-installed N-glycans (as indicated on the right side of the graphic).

FIG. 2 is a schematic representation illustrating some examples of non-natural hexosamine analogs used for conjugation applications for IgG antibodies (and other therapeutic proteins).

FIG. 3 is an image of a gel showing the production and quantification of the glycoengineered antibodies. Antibodies were expressed in HEK293 or wild-type CHO cells supplanted with the azido-analog 1,3,4-O-Bu₃ManNAz. The antibodies were separated using PAGE and the gels were analyzed by “far-western” blotting to quantify antibodies modified with azido-sialic acids.

FIG. 4 is a schematic representation illustrating an embodiment of the novel method for antibody purification. Azido-tagged IgG is ligated to alkyne-conjugated resins containing a photolabile linker. UV irradiation at 365 nm specifically releases the IgG leaving behind non-specifically bound contaminants.

FIG. 5 is a schematic representation illustrating an embodiment of the novel method for antibody purification. Vinyl phenyl tagged IgG is ligated to tetrazine-conjugated resin. The tetrazine triggers self-immolation specifically releases the IgG leaving behind non-specifically bound contaminants.

FIG. 6 is a schematic representation illustrating the components of an integrated production platform developed for the design and biomanufacturing of therapeutic proteins.

FIG. 7 is a schematic and series of depicting the glycosylation of the glycoengineered 602 variants. FIG. 7A: Threaded computational model of the heavy chain (HC) and light chain (LC) variable domains of the 602 antibody in complex with the IL-2 cytokine (generated using the iTASSER server), overlaid with the heterotrimeric IL-2 receptor (PDB ID: 2B5I). Complementarity-determining loops of the HC (H1, H2, and H3) and LC (L1, L2, and L3) are indicated (inset). Green spheres indicate the location of the asparagine residue in the engineered N-glycan site. FIG. 7B: Reducing SDS-PAGE analysis of 6 glycovariants of the 602 antibody with inserted N-linked glycosylation sites in the HC (M1 and M9) or LC (M2, M4, M8). FIG. 7C: Con-A lectin blot demonstrating glycosylation of 602 variants. Con-A Glycophorin A (GlyA) and ovalbumin (OVA) were used as controls for O-linked and N-linked glycosylation, respectively.

FIG. 8 is a series of plots depicting the binding and functional properties of the 602 glycovariants. FIG. 8A: Biolayer interferometry (BLI) studies depicting the interaction between immobilized human IL-2 and soluble antibody (either 602 or glycovariants thereof). FIGS. 8B,C: BLI-based competitive IL-2 binding studies between 602 or glycovariants thereof and IL-2 receptor subunits. Equilibrium binding of a saturating concentration of IL-2 (600 nM) to immobilized IL-2Rα (FIG. 8B) or IL-2Rβ (FIG. 8C) in the presence of titrated amounts of 602 antibody is shown. FIGS. 8D,E: IL-2 signaling pathway activation induced by a 1:1 molar ratio of 602:IL-2 complexes on human YT-1 human NK cells with (FIG. 8D) and without (FIG. 8E) IL-2Rα expression. STAT5 phosphorylation was detected via flow cytometry. Error bars represent s.d. FIG. 8F: Ratio of YT−:YT+EC₅₀ values for signaling activation by IL-2 or various IL-2/antibody complexes.

FIG. 9 is a gel (left) and a blot (right) demonstrating the increased glycosylation of the 602-M18 doubly-glycosylated antibody as indicated by ConA staining.

FIG. 10 is an in vivo pharmacokinetic study in C57BL/6J mice depicting the serum persistence of WT 602 antibody versus the M18 variant, prepared with (+) or without (−) 1,3,4-O-Bu₃ManNAc. Peripheral blood antibody concentration is plotted at various time points following retroorbital injection (n=3).

FIG. 11 is a photograph of culture plates showing transformants selection using LB agar plates containing ampicillin. FIG. 11A: Transformants with unligated PX458 plasmid. There were no obvious colonies observed. FIG. 11B: Transformants with uncut PX458 plasmid. Transformants containing undigested PX458 plasmids were used as a positive control due to the successful expression of the ampicillin resistant gene. FIG. 11C: Transformants containing the sgRNA 1 inserted vector. FIG. 11D: Transformants containing the sgRNA 2 inserted vector. There was a varied number of colonies containing each sgRNA construct present, due to different starting volumes of cell mixture seeded on each plate.

FIG. 12 is a series of sequences and plots showing the plasmid sequencing results. FIG. 12A: Sequencing results for Plasmid 1. FIG. 12B: Sequence results for Plasmid 2. FIG. 12C: Quality graph for Plasmid 1 sequencing. FIG. 12D: Quality graph for Plasmid 2 sequencing. The blue lines in FIGS. 12A and 12B represent the quality score lines and the dashed lines are the reference lines.

FIG. 13 is a series of images showing the transfection of CHO-K1 cells. FIG. 13A: Transmitted image of control cells. FIG. 13B: GFP fluorescent image of control cells. FIG. 13C: Transmitted image of Plasmid 1 transfected cells. FIG. 13D: GFP fluorescent image of Plasmid 1 transfected cells. FIG. 13E: Transmitted image of Plasmid 2 transfected cells. FIG. 13F: GFP fluorescent image of Plasmid 2 transfected cells. FIG. 13G: Transmitted image of Plasmid 1+Plasmid 2 transfected cells. FIG. 13H: GFP fluorescent image of Plasmid 1+Plasmid 2 transfected cells.

FIG. 14 is a series of FACS data analysis plots showing results from CHO-K1 cells transfected with Plasmid 1. FIG. 14A: The first gate (R1) is based on selecting cells with appropriate cell morphology. FIG. 14B: The second gate (R2) selects for single cells, as opposed to aggregates of cells. FIG. 14C: This plot depicts single cells with orange fluorescence on the y-axis and green fluorescence on the x-axis. FIG. 14D: The third gate (R3) selects for GFP positive and propidium iodide negative cells, which represent the live, transfected cells. It should be noted that the total single cell population for this condition (203,305 cells) was 10× higher in comparison to the other conditions.

FIG. 15 is a series of FACS data analysis plots showing results from CHO-K1 cells transfected with Plasmid 2. FIG. 15A: The first gate (R1) is based on selecting cells with appropriate cell morphology. FIG. 15B: The second gate (R2) selects for single cells, as opposed to aggregates of cells. FIG. 15C: This plot depicts single cells with orange fluorescence on the y-axis and green fluorescence on the x-axis. FIG. 15D: The third gate (R3) selects for GFP positive and propidium iodide negative cells, which represent the live, transfected cells. The total single cell population is 35,994 cells.

FIG. 16 is a series of FACS data analysis plots showing results from CHO-K1 cells transfected with Plasmid 1+Plasmid 2. FIG. 16A: The first gate (R1) is based on selecting cells with appropriate cell morphology. FIG. 16B: The second gate (R2) selects for single cells, as opposed to aggregates of cells. FIG. 16C: This plot depicts single cells with orange fluorescence on the y-axis and green fluorescence on the x-axis. FIG. 16D: The third gate (R3) selects for GFP positive and propidium iodide negative cells, which represent the live, transfected cells. The total single cell population is 33,595 cells.

FIG. 17 is a graph demonstrating the transfection efficiency in CHO-K1 cells. The transfection efficiency was calculated from the collected FACS data for each of the following conditions: Plasmid 1, Plasmid 2 and Plasmid 1+Plasmid 2.

FIG. 18 is a series of gels and plots showing the results obtained from gel electrophoresis. FIG. 18A: Visualization of PCR samples on 1% agarose gel. The left part of the image depicts the results of the DNA fragments containing sgRNA 1 or sgRNA 2 targeted region. The right part of the image depicts the DNA fragments containing the combination of the sgRNA 1 and the sgRNA 2 targeted region. FIG. 18B: Distance migrated by the DNA fragment from a 1 kb DNA ladder in 1% agarose gel corresponding to the left part of the image shown in FIG. 18A. Log₁₀(size)=−0.003458(migration distance)+4.802, R2=0.9947. FIG. 18C: Distance migrated by the DNA fragment from a 1 kb DNA ladder in 1% agarose gel corresponding to the right part of the image shown in FIG. 18A. Log₁₀(size)=−0.003448(migration distance)+4.727, R2=0.9821.

FIG. 19 is a series of plots and a table demonstrating the genomic editing efficacy of sgRNA 1. FIG. 19A: Point mutations for Plasmid 1 samples. FIG. 19B: Point mutations for combined Plasmid 1+Plasmid 2 samples. FIG. 19C: Deletion (red), insertion (blue) and mismatch (yellow) locations for sgRNA 2 samples. FIG. 19D: Representative quality of Sanger sequencing (sample 1 shown) with the target region outlined in red.

FIG. 20 is a series of plots and a table demonstrating the genomic editing efficacy of sgRNA 2. FIG. 20A: Point mutations for Plasmid 2 samples. FIG. 20B: Point mutations for combined Plasmid 1+Plasmid 2 samples. FIG. 20C: Deletion (red), insertion (blue) and mismatch (yellow) locations for sgRNA 2 samples. N88 is an 88-nucleotide insertion. FIG. 20D: Representative quality of Sanger sequencing (sample 5 shown) with the target region outlined in red.

FIG. 21 provides genetic analysis of indel generation in CHO^{Gne-} cells. The region on exon 2 and exon 4 of Gne targeted by the gRNA was amplified and cloned into an empty vector. Plasmid was isolated from 20 bacteria colonies (10 per gRNA target site) and Sanger sequenced. (A) For the exon 2 site, all plasmids showed deletion of one thymine residue five base pairs upstream of the PAM motif. (B) For the exon 4 site, all plasmids showed insertion of one adenine residue five base pairs upstream of the PAM motif. The quality score graph and chromatogram are shown for one representative plasmid for each target site and the MUSCLE alignment of all plasmids with Gne is show on the bottom. The gRNA is highlighted in yellow, the PAM sequence is highlighted in green and the gene edit is highlighted in red.

FIG. 22 provides genetic analysis of indel generation in CHO^{Renhp-} cells. The region on exon 3 and exon 6 of Renbp targeted by the gRNA was amplified and cloned into an empty vector. Plasmid was isolated from 20 bacteria colonies (10 per gRNA target site) and Sanger sequenced. (A) For the exon 3 site, nine plasmids showed insertion of one thymine residue while one plasmid showed insertion of a cytosine residue at the same location. (B) For the exon 6 site, nine plasmids showed insertion of one thymine residue, while one plasmid showed insertion of a cytosine residue at the same location. The quality score graph and chromatogram are shown for one representative plasmid for each target site and the MUSCLE alignment of all plasmids with Renbp is show on the bottom. The gRNA is highlighted in yellow, the PAM sequence is highlighted in green and the gene edit is highlighted in red.

FIG. 23 shows the glycosylation and sialylation of secreted proteins from CHO-K1 and CHO^{Gne-} cells. (left panel) Commassie blue stained gel showing proteins secreted from the two CHO cell lines in a 24 hour period after transfer to serum free medium. (center panel) Con A lectin blot showing the overall glycosylation of the secreted proteins. (right panel) Mal II lectin blot showing the α2,3-sialylation of the proteins. The samples were alternatively treated (+) or not treated (−) with 1,3,4-O-Bu₃ManNAz, as indicated at the top of the gels.

FIG. 24 shows the expression of ST6GAL1 in CHO^{Gne-} cells. The parent CHO-K1 cells express endogenous Gne (left lane) but not ST6GAL1 (which is a human gene); the second lane show successful knock out of Gne expression in the CHO^{Gne-} cells. The third lane shows neo-expression of ST6GAL1 in the parent cells and the rightmost lane shows ST6GAL expression in the CHO^{Gne-} cells.

FIG. 25 is a schematic and a series of gels depicting the glycosylation of the glycoengineered F5111 variants. FIG. 25A: Crystallographic structure of the heavy chain (HC) and light chain (LC) variable domains of the F5111 antibody in complex with the IL-2 cytokine (PDB ID: 5UTZ), overlaid with the heterotrimeric IL-2 receptor (PDB ID: 2B5I). Complementarity-determining loops of the F5111 HC (H1, H2, and H3) and LC (L1, L2, and L3) are indicated (inset). Green spheres indicate the location of the asparagine residue in the engineered N-glycan site. FIG. 25B: Reducing SDS-PAGE analysis of six glycovariants of the F5111 antibody with inserted N-linked glycosylation sites in the HC (M1, M2, and M3) or LC (M4, M5, and M6). FIG. 25C: Con-A lectin blot demonstrating glycosylation of F5111 variants.

FIG. 26 is a series of plots depicting the binding and functional properties of the F5111 glycovariants. FIG. 26A: Biolayer interferometry (BLI) studies depicting the interaction between immobilized human IL-2 and soluble antibody (either F5111 or glycovariants thereof). FIGS. 26B-C: Competitive BLI-based IL-2 binding studies between F5111 or glycovariants thereof and IL-2 receptor subunits. Equilibrium binding of a saturating concentration of IL-2 (600 nM) to immobilized IL-2Rα (FIG. 21B) or IL-2Rβ (FIG. 26C) in the presence of titrated amounts of F5111 antibody. FIGS. 26D,E: IL-2 signaling pathway activation induced by a 1:1 molar ratio of F5111:IL-2 complexes on human YT-1 human NK cells with (FIG. 26D) and without (FIG. 26E) IL-2Rα expression. STAT5 phosphorylation was detected via flow cytometry. Error bars represent s.d. (FIG. 26F) Comparison of YT−:YT+EC₅₀ values for signaling activation by IL-2 or various IL-2/antibody complexes.

FIG. 27 shows schematic representations showing the structural considerations for protein design. FIG. 27A: ENPP1 glycosylation sites. Known glycosylation sites map onto random coil regions (Green spheres=N-acetylglucosamine) of PDB 4GTW. One additional glycosylation site is located in the α helix shown as a green Asn residue and subsequently confirmed in PDB 6AEK. There are five additional consensus sequences (Asn Leu Thr) found in human ENPP1 whose glycosylation status is unknown. Calcium atom in orange; 2 zinc atoms are in Cyan; ATP is in Yellow; residue in red is located in the catalytic domain and corresponds to V246D mutation in ASJ mice; catalytic domain in blue and endonuclease domain in purple. FIG. 27B: Loss of function mutations resulting in GACI. The same model as in FIG. 27a, without glycan spheres, showing the locations of GACI point mutations in green. FIG. 27C: RaptorX model of ENPP1-Fc Cyan residues: N-terminal signal sequence from hENPP7; Green residues: somatomedin B domain-1; Yellow residues: somatomedin B domain-2; Blue residues: catalytic domain; Purple residues: endonuclease domain; Red residues: Fc domain (from human IgG1). In this model the green spheres represent known glycans chains, including the known glycan in the Fc domain, and the pink spheres represent the novel glycan at residue Asn254 which bestows enhanced pharmacokinetic properties on the enzyme.

FIG. 28 is a series of graphs, plots and a schematic representation demonstrating the pharmacokinetic effects of additional N-Glycans. FIG. 28A: Domain structure of the parent clone 770. The 2 somatomedin B domains (green and yellow), catalytic domain (blue) and endonuclease domain (purple) of human ENPP1 was fused N-terminally with the signal sequence of human ENPP7 (blue) and c-terminally with the Fc domain of human IgG1 (red). FIG. 28B: Pharmacokinetic analysis of the parent clone 770, with fractional activity fitted to curve described in Equation 1. The fractional activity of 770 in the blood was sampled in each mouse at four separate times over 200 hours (individual data overlaid in green). Five curves fitted representing the clearance of 770 in each mouse according to Equation 1 are overlayed, from which relevant PK constants are obtained. FIG. 28C: Pharmacokinetic effects of additional N-glycosylation consensus sequences engineered into the parent clone 770 represented by overlays of area under the curve (AUC, left y axis) and half-life (right y-axis). Individual measurements of AUC in each animal is plotted as blue points, with the means represented by cyan bars, with error bars representing standard deviation. Individual half-life measurements are plotted in red, with the means and standard deviations shown. Clone 7, with the I256T mutation, has a marked increase in both AUC and half-life compared with parent clone 770. FIG. 28D: Steady state Michaelis-Menten assays as two separate concentrations comparing the kinetics of 770 (in black) with clones possessing the 1256T mutation (clones 17 in yellow and 19 in red). Each point represents the average of six measurements of each enzyme at various substrate concentrations. Error bars denote standard deviations of the mean.

FIG. 29 is a mass spectrometry analysis of sialic acid content on residue 254. i and iii. Trypsinized ENPP1-Fc clones 770 and 19, respectively, analyzed by LC-MS/MS. ii and iv. detail of the digested peptide fragment ²⁴¹SGTFFWPGSDVEINGTFPDIYK²⁶² demonstrating an abundance of sialylated glycopeptide peaks in ENPP1-Fc clone 19 (panel iv) which are absent in the parent clone 770 (panel ii).

FIG. 30 is a series of graphs and plots demonstrating the pharmacokinetic effects of combining addition of N-glycan consensus sequons, Fc mutations, and Glyco-polishing. FIG. 30A: Pharmacokinetic effects of Fc mutations—overlays of area under the curve (left y axis) and half-life (right y-axis). The data is displayed as in FIG. 28C. Clones containing the Fc-HN mutation are clones 9, 10, 11, 12 and 15, and clones containing the Fc-MST mutation are clones 8, 13, 14, 16, and 17. These are compared with the parent clone (770) and clone 7 containing only the I256T mutation. FIG. 30B: Biological availability of clones 14, 7, and 19, highlighting the AUC to visualize changes induced by protein engineering techniques. Individual data points display the means and standard deviations at the listed time points (individual data displayed in FIGS. 30A and 30C). Clone 7, which contains only the I256T mutation, exhibits the highest initial activity (Cmax) but tapers off quickly (red area). Clone 14, which contains only the Fc MST mutation, has a lower Cmax but longer half-life than clone 7, as seen by shallow slope (gray area). Combining both mutations into a single clone—19(ST)—results in an enzyme with a greater initial activity and a longer half-life (yellow area) FIG. 30C: Pharmacokinetic effects of glycopolishing represented by overlays of area under the curve (AUC, left y axis) and half-life (right y-axis). PK constants of glycoforms expressed in unmodified CHO cells—clone numbers only—are compared to those expressed in CHO cells over-expression in α-2,6-sialytransferase (α-2,6-ST) with or without 1,3,4-O-Bu₃ManNAc supplementation—clone numbers followed by ST or STA, respectively. Data is represented as in FIG. 28C. In general, production in α-2,6-ST-expressing cells was beneficial to half-life and AUC, and growing the clones in sialic acid precursors provided further benefit. For example, clone 9 exhibits a stepwise increase in AUC and half-life following expression in α-2,6-ST-expressing CHO cells and 1,3,4-O-Bu₃ManNAc supplementation (cyan arrows). 1256T bearing glycoforms (red arrows) exhibited a similar progression. FIG. 30D: Anion-Exchange Chromatography with Pulsed Amperometric Detection (HPAE-PAD) of clone 9 grown in CHO K1 cells alone or stably transfected with human α-2,6-ST or a combination of α-2,6-ST and the sialic acid precursor 1,3,4-O-Bu₃ManNAc shows a progressive increase in the percentage N-acetylneuraminic acid content with each treatment. FIG. 30E: Biological availability plots, displayed as in FIG. 28B, comparing clone 770 with glycopolished forms of clone 9 to demonstrate the progressive effects of glycopolishing on AUC. Data points represent the means and standard deviation at the listed times (individual data displayed in FIG. 30C).

FIG. 31 is a series of graphs demonstrating the pharmacokinetic and pharmacodynamic effects of optimized ENPP1-Fc. FIG. 31A: Biological availability plots comparing Fc mutated clones without (clone 14(ST)) and with the 1256T mutation and further glycopolishing (clones 17(ST), 19(ST) and 19(ST)A). Data points represent the means and standard deviations of data individually displayed in FIG. 31C. The AUC for Fc-MST containing clones (shaded area) was enhanced by the 1256T mutation and further increased by 1,3,4-O-Bu₃ManNAc supplementation. FIG. 31B: Biological availability curves comparing the parent clone (770) with the final optimized glycoform (clone 19(ST)A), demonstrating a nearly 18-fold increase in bioavailability in the final product. Data points represent the means and standard deviations of data individually shown in FIG. 30C. FIG. 31C: MALDI-TOF/TOF analysis for N-glycan profiling revealed that the % glycans containing sialic acid is higher in clone 19(ST)A (99.2%) compared to parent clone 770 (78.4%) when calculated based on the structures that contains at least one galactose for transfer of sialic acid. FIG. 31D: The pharmacodynamic effect after a single dose at 0.3 mg/kg of either the parent clone 770 (red squares) or the optimized ENPP1-Fc clone 19(ST) (red circles), as measured by plasma [PPi] (left y-axis) in Enpp1^{asj/asj} mice. Physiological levels of PPi in normal mice (shaded grey) is between 1.5 and 2.5 μM PPi while Enpp1^{asj/asj} mice have nearly undetectable amounts. A single dose of clone 770 restores physiological levels of PPi that return to baseline in less than 89 hours while clone 19(ST) maintained or exceeded physiological levels for 263 hours. Data points represent the mean and standard deviation of 5 animals. FIG. 31E: Plasma [PPi] 770 in Enpp1^{asj/asj} mice before and after dosing animals with 7.5 mg/kg per week with clone 770 in 3 equally divided doses (on Monday, Wednesday, and Friday). Box and violin plots represent individual data points with min to max distribution. **p<0.01, Student's paired T-test.

FIG. 32 is a schematic representation showing a summary of the protein engineering steps on (FIG. 32A) Half Life and (FIG. 32B) Area under the Curve.

### DETAILED DESCRIPTION

The disclosure provides for isolated host cells which lack one or more enzymes that regulate sialic acid metabolic flux enzymes and/or overexpress one or more enzymes that regulate N-glycan branching, e.g. N-acetylglucosaminyltransferases, and/or one or more enzymes that regulate sialylation comprise sialyltransferases. These cells are used as part of an integrated production platform developed for the design and biomanufacturing of glycoengineered proteins. These cells can be cultured in media comprising one or more high-flux sugar analogs.

Biochemically, N-glycan branching beyond the canonical biantennary type N-glycan requires two components. The first is the presence of the requisite GNT4/5 (human) or Gnt4/5 (rodent) enzymes coded by the MGAT4/5 (or Mgat4/5) genes. GNT/Gnt4 installs the GlcNAc residue needed for triantennary N-glycans and GNT/Gnt5 installs the GlcNAc residue needed for tetraantennary N-glycans. A key aspect of GNT4/5 activity is the requirement for higher than naturally-occurring levels of UDP-GlcNAc, which is the substrate for these enzymes. Cellular levels of UDP-GlcNAc typically are 0.1 to 1.0 mM, but the Kₘ for GNT4 is ˜5 mM and for GNT5 is ˜11 mM, meaning that these enzymes are usually minimally active in cells including CHO cells used in biomanufacturing. One way to overcome this pitfall is through supplementation of the culture medium with GlcNAc, however this approach is not practical for biomanufacturing because of the high concentrations (e.g., 10 to 50 mM) of this sugar needed to increase intracellular levels of UDP-GlcNAc. Problems with using such high levels range from scientific (e.g., osmotic shock that decreases cell viability) to the economic.

Accordingly, this disclosure provides for modified host cells which are advantageous for many reasons, including the ability to produce glycoproteins which have been site specifically modified to with N-glycans. In certain embodiments a modified host cell comprises one or more nucleic acid sequences encoding one or more enzymes that modulate N-glycan branching, sialylation or a combination thereof. In certain embodiments, the host cell is transformed with one or more vectors comprising one or more nucleic acid sequences encoding for one or more enzymes are expressed by a vector enzymes that modulate N-glycan branching, sialylation or combinations thereof. The one or more enzymes that modulate N-glycan branching comprise N-acetylglucosaminyltransferases. The one or more enzymes that modulate sialylation comprise sialyltransferases.

Certain embodiments include host cells that have been manipulated to inhibit expression, function or activity of one or more enzymes that modulate sialic acid metabolic flux. This can be accomplished by various methods including gene-editing. For example, the host cells are contacted with a gene-editing agent which inactivates regulatory or other components that encode for these enzymes or by using a multiplex approach whereby the gene editing agent is targeted at both ends of the nucleic acid segment to be excised. In certain embodiments, the one or more enzymes that modulate sialic acid metabolic flux comprise GNE (UDP-N-acetylglucosamine 2-epimerase/N-acetylmannosamine kinase), RENBP (Renin-binding protein; N-GlcNAc 2-epimerase), NPL (N-acetylneuraminic acid lyase), NEU1 (Neurimindase 1), NEU2 (Neurimindase 2), NEU3 (Neurimindase 3), NEU4 (Neurimindase 4), Klotho or combinations thereof.

GNE (UDP-N-acetylglucosamine 2-epimerase N-acetylmannosamine kinase; EC 3.2.1.183) GNE catalyzes the first two “committed” steps in the sialic acid biosynthetic pathway, which are (i) conversion of UDP-GlcNAc to ManNAc and (ii) conversion of ManNAc to ManNAc-1-P. This enzyme has been described as the key regulator of flux into the sialic acid pathway and downstream sialylation. This prevents the flux of natural metabolites (i.e., natural ManNAc) though the sialic acid pathway, which enables an increase in flux of non-natural analogs (e.g., an azido-modified ManNAz analog) through the pathway and into the glycans of therapeutic proteins.

RENBP (Renin-binding protein; N-GlcNAc 2-epimerase; EC 5.1.3.8). RENBP was first characterized to bind to and modulate renin. Later is was found to have GlcNAc-2-epimerase activity; i.e., it interconverts GlcNAc and ManNAc. Since RENBP can produce ManNAc, it can feed this sugar into the sialic acid pathway and competitively inhibit the flux of our non-natural ManNAc analogs through the pathway.

NPL (N-acetylneuraminic acid lyase; FC 4.1.3.3). This enzyme negates sialic acid synthesis (i.e., it converts sialic acid to ManNAc and pyruvate).

NEU1, NEU2, NEU3, and NEU4 (Neurimindase 1, 2, 3 and 4; E.C. 3.2.1.18). Mammals have four main enzymes that remove sialic acid from glycoconjugates (NEU1, NEU2, NEU3, and NEU4) as well as Klotho, a senescence suppressing protein that also has neuraminidase activity.

Gene-editing agents: Embodiments include compositions for the inactivation or deletion of genes encoding for the enzymes which modulate sialic acid metabolic flux. Methods of the invention may be used to remove genetic material from a host organism, without interfering with the integrity of the host's genetic material. A nuclease may be used to target enzymes which modulate sialic acid metabolic flux, thereby interfering with replication or transcription or even excising the desired genetic material from the host genome. Targeting the enzyme nucleic acid sequences can be done using a sequence-specific moiety such as a guide RNA that targets the desired genomic material for destruction by the nuclease and does not target the host cell genome. In some embodiments, a CRISPR/Cas nuclease and guide RNA (gRNA) that together target and selectively edit or destroy nucleic acid sequences encoding for enzymes which modulate sialic acid metabolic flux genomic material is used. The CRISPR (clustered regularly interspaced short palindromic repeats) is a naturally-occurring element of the bacterial immune system that protects bacteria from phage infection. The guide RNA localizes the CRISPR/Cas complex to an enzyme target nucleic acid sequence. Binding of the complex localizes the Cas endonuclease to the genomic target sequence causing breaks in the targeted segment of the genome. Other nuclease systems can be used including, for example, zinc finger nucleases, transcription activator-like effector nucleases (TALENs), meganucleases, or any other system that can be used to degrade or interfere with target nucleic acid without interfering with the regular function of the host's genetic material.

CRISPR Cas Systems: The CRISPR-Cas system includes a gene editing complex comprising a CRISPR-associated nuclease, e.g., Cas9, and a guide RNA complementary to a target sequence situated on a DNA strand, such as a target sequence in one or more sequences encoding enzymes that modulate sialic acid metabolic flux. The gene editing complex can cleave the DNA within the target sequence. This cleavage can in turn cause the introduction of various mutations into the target nucleic acid sequences, resulting in inactivation or silencing of the targeted gene. The mechanism by which such mutations inactivate the target DNA can vary. For example, the mutation can affect gene expression. The mutations may be located in regulatory sequences or structural gene sequences and result in defective production of the enzyme. The mutation can comprise a deletion. The size of the deletion can vary from a single nucleotide base pair to about 10,000 base pairs. In some embodiments, the deletion can include all or substantially all of the target nucleic acid sequence. In some embodiments the deletion can include the entire target nucleic acid sequence. The mutation can comprise an insertion, that is, the addition of one or more nucleotide base pairs to the target sequence. The size of the inserted sequence also may vary, for example from about one base pair to about 300 nucleotide base pairs. The mutation can comprise a point mutation, that is, the replacement of a single nucleotide with another nucleotide. Useful point mutations are those that have functional consequences, for example, mutations that result in the conversion of an amino acid codon into a termination codon or that result in the production of a nonfunctional protein.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems (Burstein, D. et al. New CRISPR-Cas systems from uncultivated microbes. Nature. 2017 Feb. 9; 542(7640):237-241. doi: 10.1038/nature21059. Epub 2016 Dec. 22).

In embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In embodiments, the CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity”S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The disclosure is by no means limited to these variants, and also encompasses all Cas9 variants (Slaymaker, I. M. et al. Science. 2016 Jan. 1; 351(6268):84-8. doi: 10.1126/science.aad5227. Epub 2015 Dec. 1). The present invention also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

Embodiments also include a new type of class 2 CRISPR-Cas system found in the genomes of two bacteria recovered from groundwater and sediment samples. This system includes Cas1, Cas2, Cas4 and an approximately ˜ 980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2017 supra).

Another new class 2 Cas protein is encoded in the genomes of certain candidate phyla radiation (CPR) bacteria. This approximately 1,200 amino acid Cas protein, termed CasY, appears to be part of a minimal CRISPR-Cas system that includes Cas1 and a CRISPR array. Most of the CRISPR arrays have unusually short spacers of 17-19 nt, but one system, which lacks Cas1 (CasY.5), has longer spacers (27-29 nt). Accordingly, in some embodiments of the invention, the CasY molecules comprise CasY.1, CasY.2, CasY.3, CasY.4, CasY.5, CasY.6, mutants, variants, analogs or fragments thereof.

In some embodiments, the CRISPR/Cas-like protein can be derived from a wild type Cas protein or fragment thereof. In other embodiments, the CRISPR/Cas-like protein can be derived from modified Cas proteins. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In some embodiments, the CRISPR-associated endonuclease can be a sequence from another species, for example, other bacterial species, bacteria genomes and archaea, or other prokaryotic microorganisms. Alternatively, the wild type Cas9, CasX, CasY.1, CasY.2, CasY.3, CasY.4, CasY.5, CasY.6, ARMAN 1, ARMAN 4, sequences can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in GENBANK accession numbers KM099231.1 GI:669193757; KM099232.1 GI:669193761; or KM099233.1 GI:669193765. Alternatively, the Cas9, CasX, CasY.1, CasY.2, CasY.3, CasY.4, CasY.5, CasY.6, ARMAN 1, ARMAN 4, sequences can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of GENBANK accession numbers KM099231.1 GI:669193757; KM099232.1 GI:669193761; or KM099233.1 GI:669193765, or Cas9 amino acid sequence of PX330 or PX260 (Addgene, Cambridge, MA).

The wild type Cas9, CasX, CasY.1, CasY.2, CasY.3, CasY.4, CasY.5, CasY.6, ARMAN 1, ARMAN 4, sequences can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The sequences of Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, can be modified to encode biologically active variants, and these variants can have or can include, for example, an amino acid sequence that differs from a wild type by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9, CasX, CasY.1, CasY.2, CasY.3, CasY.4, CasY.5, CasY.6, spCas, eSpCas, SpCas9, ARMAN 1, ARMAN 4 polypeptides. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

Guide RNA: A gRNA includes a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). In the present disclosure, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from U6 or H1-promoted RNA expression vector.

In the compositions of the present invention, each gRNA includes a sequence that is complementary to a target sequence. Examples are shown in Tables 1 and 2.

Guide RNA sequences can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the target sequence. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. Combinations of gRNAs are especially effective when expressed in multiplex fashion, that is, simultaneously in the same cell.

### Interleukin-2 Modulatory Antibodies

Interleukin-2 (IL-2) is a multi-functional cytokine that orchestrates the differentiation, proliferation, survival, and activity of immune cells. Due to its potent activation of the immune response, high-dose IL-2 therapy has been used clinically to stimulate anti-cancer immunity and received FDA approval for treatment of metastatic renal cell carcinoma in 1992 and for metastatic melanoma in 1998 (Liao W, Lin J-X, Leonard W J. Interleukin-2 at the crossroads of effector responses, tolerance, and immunotherapy. Immunity. 2013 Jan. 24; 38(1): 13-25. PMCID: PMC3610532). By activating the patient's own immune system, IL-2 therapy elicits complete and durable responses in 5-10% of patients (Rosenberg S A. Raising the bar: the curative potential of human cancer immunotherapy. Sci Transl Med. 2012 Mar. 28; 4(127): 127ps8. PMID: 22461638). However, IL-2 simultaneously activates both immune effector cells (Effs) and regulatory T cells (T_{RegS}), limiting efficacy and resulting in harmful off-target effects and toxicities, most prominently severe vascular leak syndrome, which can lead to edema, organ failure, and death (Boyman O, Sprent J. The role of interleukin-2 during homeostasis and activation of the immune system. Nat Rev Immunol. 2012 Feb. 17; 12(3): 180-190. PMID: 22343569; Dhupkar P, Gordon N. Interleukin-2: Old and New Approaches to Enhance Immune-Therapeutic Efficacy. Adv Exp Med Biol. 2017; 995:33-51. PMID: 28321811). Furthermore, the vanishingly short serum half-life (<5 min) of IL-2 hinders its clinical performance (Donohue J H, Rosenberg S A. The fate of interleukin-2 after in vivo administration. J Immunol Baltim Md 1950. 1983 May; 130(5):2203-2208. PMID: 6601147). There is thus an unmet clinical need for a therapeutic agent that safely and selectively activates immune effector cells directly in patients.

A breakthrough study in 2006 demonstrated that complexes of IL-2 with the anti-IL-2 antibody S4B6 selectively bias cytokine activity toward Effs (i.e. memory phenotype CD8⁺ Eff T cells and natural killer [NK] cells) while concurrently extending the in vivo half-life of IL-2 to elicit targeted, durable immunostimulatory responses (Boyman O, Kovar M, Rubinstein M P, Surh C D, Sprent J. Selective stimulation of T cell subsets with antibody-cytokine immune complexes. Science. 2006 Mar. 31; 311(5769): 1924-1927. PMID: 16484453). The IL-2/S4B6 complex structure was determined, elucidating the molecular mechanism through which S4B6 capitalizes on differential immune cell sensitivity to IL-2 to bias cytokine activity.⁷ IL-2 activates cell signaling through either a high-affinity (K_{D}˜10 pM) heterotrimeric receptor consisting of the IL-2 receptor-α (IL-2Rα), IL-2Rβ, and common gamma (γ_{c}) chains, or an intermediate-affinity (K_{D}˜1 nM) heterodimeric receptor consisting of only the IL-2Rβ and Ye chains. Consequently, IL-2 responsiveness is determined by the IL-2Rα subunit, which is highly expressed on T_{RegS}, but virtually absent from naïve Effs, rendering T_{RegS} 100-fold more sensitive to IL-2 (Boyman, O et al., 2012; Malek T R. The Biology of Interleukin-2. Annu Rev Immunol. 2008; 26(1):453-479. PMID: 18062768; Spangler J B, Moraga I, Mendoza J L, Garcia K C. Insights into Cytokine-Receptor Interactions from Cytokine Engineering. Annu Rev Immunol. 2015 Mar. 21; 33(1):139-167). S4B6 binding sterically blocks the IL-2/IL-2Rα interaction but also allosterically enhances the IL-2/IL-2Rβ interaction, eliminating the IL-2 sensitivity advantage of T_{RegS} and simultaneously potentiating IL-2 activity to preferentially expand Eff versus T_{Reg} cells and mount an immunostimulatory response (Spangler J B, et al. Antibodies to Interleukin-2 Elicit Selective T Cell Subset Potentiation through Distinct Conformational Mechanisms. Immunity. 2015 May; 42(5): 815-825).

Antibody-mediated immune bias presents an exciting opportunity for targeted cytokine therapy. Indeed, IL-2/S4B6 complexes induce potent anti-tumor activity in mice in the absence of adverse effects typically associated with systemic IL-2 administration (Verdeil G, et al., Adjuvants targeting innate and adaptive immunity synergize to enhance tumor immunotherapy. Proc Natl Acad Sci USA. 2008 Oct. 28; 105(43):16683-16688. PMCID: PMC2575480; Jin G-H, Hirano T, Murakami M. Combination treatment with IL-2 and anti-IL-2 mAbs reduces tumor metastasis via NK cell activation. Int Immunol. 2008 Jun. 1; 20(6):783-789; Krieg C, et al., Improved IL-2 immunotherapy by selective stimulation of IL-2 receptors on lymphocytes and endothelial cells. Proc Natl Acad Sci. 2010 Jun. 29; 107(26):11906-11911. PMID: 20547866). However, S4B6 recognizes mouse IL-2 (mIL-2) and has limited cross-reactivity with human IL-2 (hIL-2). Recently, an anti-hIL-2 analog of S4B6 (denoted 602) was isolated, and it was reported that treatment with hIL-2/602 antibody complexes also preferentially expands immune effector cells and inhibits tumor growth in mice; thus, antibody 602 has potential as an anti-cancer therapeutic. Based on its potential therapeutic uses, in this disclosure we use antibody 602 as a lead example of how our glycoengineering platform can improve therapeutic proteins but emphasize that this technology broadly applies to virtually all proteins; for example additional instances described below include a second IL-2 modulatory antibody (F5111 in Example 4) and a therapeutic enzyme (ENPP-1 in Example 5).

### Glycoengineering of Therapeutic Proteins

Sugar chains of glycoproteins are roughly divided into two types, namely a sugar chain which binds to asparagine (N-glycoside-linked sugar chain) and a sugar chain which binds to other amino acid such as serine, threonine (O-glycoside-linked sugar chain), based on the binding form to the protein moiety.

The sugar chain terminus which binds to asparagine is called a reducing end, and the opposite side is called a non-reducing end. It is known that the N-glycoside-linked sugar chain includes a high mannose type in which mannose alone binds to the non-reducing end of the core structure; a complex type in which the non-reducing end side of the core structure has at least one parallel branches of galactose-N-acetylglucosamine (hereinafter referred to as “Gal-GlcNAc”) and the non-reducing end side of Gal-GlcNAc has a structure of sialic acid, bisecting N-acetylglucosamine or the like; a hybrid type in which the non-reducing end side of the core structure has branches of both of the high mannose type and complex type.

The sugar chain structure play important roles for the structure, function, size, circulatory half-life, and pharmacokinetic behavior of glycoprotein drugs. Moreover, the sugar structure plays a remarkably important role in the effector function of antibodies and differences are observed in the sugar chain structure of glycoproteins expressed by host cells, development of a host cell which can be used for the production of an antibody having higher effector function is desired.

Monoclonal antibodies (MAbs) have emerged as effective biopharmaceuticals for cancer and other chronic diseases due to the specificity of these drugs toward target antigens, for example, by activating the immune system to kill tumor cells, blocking the signal transduction of tumor cells to proliferate, carrying drugs to tumor cells or as radiation targets. The glycosylation of immunoglobulins influences both their physiochemical properties, and their cell-mediated effector functions such as complement binding and activation. These biological functions may be dependent not only on the presence or absence of N-linked oligosaccharides but also on the specific structure of the oligosaccharides. Furthermore, N-glycosylation of antibodies is routinely characterized in the manufacturing of biopharmaceuticals. In particular, the glycan profile of a monoclonal antibody is sometimes defined as a critical quality attribute, since it can be a measure of efficacy, immunogenicity, and manufacturing conditions.

Accordingly, in certain embodiments, an engineered protein comprises one or more modified amino acid sequences, wherein the modified amino acid sequences comprise one or more N-glycan consensus sequences. In certain embodiments, the N-glycan consensus sequence comprises Asn-X-Ser/Thr, where X is any amino acid except proline (Pro). In certain embodiments, the engineered protein comprises one or more hexosamine analogs. In certain embodiments, the engineered protein further comprises one or more functional groups and/or non-functional groups. In certain embodiments, a functional group comprises: an azido group, thiol group, alkyne, alkyl, alkenyl, alkynyl, carboxamido, aldehydes or combinations thereof.

In certain embodiments, the host cells transformed with a vector encoding the engineered protein of interest. The protein of interest can be various types of protein, and in particular proteins that benefit from being expressed as glycoproteins. Examples include, erythropoietin (EPO), α1-antitrypsin, a recombinant blood factor, such as factor VIII, factor IX, factor XIII A-subunit, thrombin, factor VIIa. Other examples, include a recombinant thrombolytic, anticoagulant or another blood-related product, such as tissue plasminogen activator (tPA), hirudin, antithrombin, plasmin, plasma kallikrein inhibitor, and activated protein C. Another example is arecombinant hormone, such as insulin, insulin degludec, human growth hormone, somatropin, pegvisomant, follicle-stimulating hormone, follitropin alfa, corifollitropin alfa, follitropin beta, metreleptin, liraglutide, parathyroid hormone, lutropin, teriparatide, nesiritide, and glucagon. Another example, a recombinantgrowth hormone, such as, EPO, filgrastim, sargramostim, mecaserim, and palifermin. The agents can be cytokines, such as, interferons, interleukins, tumor necrosis factor and the like. The interferoncan include pegylated interferons.

In certain embodiments, the protein is a fusion protein comprising an Fc region is a composition in which an antibody comprising the Fc region of an antibody or the antibody fragment is fused with a protein such as an enzyme, a cytokine or the like.

In certain embodiments, the protein is an antibody, an antibody fragment, a chimeric antibody, a fusion protein comprising anFc region, and the like. A human chimeric antibody is an antibody which comprises an antibody heavy chain variable region (hereinafter referred to as “VH”, the heavy chain being “H chain”) and an antibody light chain variable region (hereinafter referred to as “VL”, the light chain being “L chain”), both of an animal other than human, a human antibody heavy chain constant region (hereinafter also referred to as “CH”) and a human antibody light chain constant region (hereinafter also referred to as “CL”). As the animal other than human, any animal such as mouse, rat, hamster, rabbit or the like can be used, so long as a hybridoma can be prepared therefrom.

In certain embodiments, the antibody is an IgG or other class (e.g., IgE or IgM) antibody or anyone of the subclasses belonging to the IgG class, such as IgG1, IgG2, IgG3 and hgG4, can be used.

In certain embodiments, the antibody is an antibody which recognizes a tumor-related antigen, an antibody which recognizes an allergy- or inflammation-related antigen, an antibody which recognizes circulatory organ disease-related antigen, an antibody which recognizes an autoimmune disease-related antigen or an antibody which recognizes a viral or bacterial infection-related antigen.

In one embodiment, the antibody or antibody composition has a high antibody-dependent cell-mediated cytotoxic activity (ADCC) activity as compared to an unmodified antibody counterpart. ADCC activity is a cytotoxic activity in which an antibody bound to a cell surface antigen on a tumor cell in the living body activate an effector cell through an Fc receptor existing on the antibody Fc region and effector cell surface and thereby obstruct the tumor cell and the like. An antibody having potent antibody-dependent cell-mediated cytotoxic activity is useful for preventing and treating various diseases including cancers, inflammatory diseases, immune diseases such as autoimmune diseases, allergies and the like, circulatory organ diseases or viral or bacterial infections.

In the case of cancers, namely malignant tumors, cancer cells grow. General anti-tumor agents inhibit the growth of cancer cells. In contrast, an antibody having potent antibody-dependent cell-mediated cytotoxic activity can treat cancers by injuring cancer cells through its cell killing effect, and therefore, it is more effective as a therapeutic agent than the general anti-tumor agents.

Cancer to be treated can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

In immune diseases such as inflammatory diseases, autoimmune diseases, allergies and the like, in vivo reactions of the diseases are induced by the release of a mediator molecule by an immune effector cell.

Exemplary inflammatory or autoimmune disorders or diseases that may be treated with the present compositions include for instance systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

Pharmaceutical compositions comprising the antibody or protein composition can be administered as a therapeutic agent alone, but generally, it is preferable to provide it as a pharmaceutical formulation produced by an appropriate method well known in the technical field of manufacturing pharmacy, by mixing it with at least one pharmaceutically acceptable carrier.

It is desirable to select a route of administration which is most effective in treatment. Examples include oral administration and parenteral administration, such as buccal, tracheal, rectal, subcutaneous, intramuscular, intravenous or the like. In an antibody preparation, intravenous administration is preferable.

The dosage form includes sprays, capsules, tablets, granules, syrups, emulsions, suppositories, injections, ointments, tapes and the like.

Examples of the pharmaceutical preparation suitable for oral administration include emulsions, syrups, capsules, tablets, powders, granules and the like.

Liquid preparations, such as emulsions and syrups, can be produced using, as additives, water; saccharides, such as sucrose, sorbitol, fructose, etc.; glycols, such as polyethylene glycol, propylene glycol, etc.; oils, such as sesame oil, olive oil, soybean oil, etc.; antiseptics, such as p-hydroxybenzoic acid esters, etc.; flavors, such as strawberry flavor, peppermint, etc.; and the like.

Capsules, tablets, powders, granules and the like can be produced using, as additive, fillers, such as lactose, glucose, sucrose, mannitol, etc.; disintegrating agents, such as starch, sodium alginate, etc.; lubricants, such as magnesium stearate, talc, etc.; binders, such as polyvinyl alcohol, hydroxypropylcellulose, gelatin, etc.; surfactants, such as fatty acid ester, etc.; plasticizers, such as glycerine, etc.; and the like.

Examples of the pharmaceutical preparation suitable for parenteral administration include injections, suppositories, sprays and the like.

Injections may be prepared using a carrier, such as a salt solution, a glucose solution, a mixture of both thereof or the like. Also, powdered injections can be prepared by freeze-drying the antibody or protein composition in the usual way and adding sodium chloride.

Suppositories may be prepared using a carrier such as cacao butter, hydrogenated fat, carboxylic acid or the like.

Also, sprays may be prepared using the antibody or peptide compositions as such, or using a carrier which does not stimulate the buccal or airway mucous membrane of the patient and can facilitate absorption of the antibody or protein composition by dispersing it as fine particles.

Examples of the carrier include lactose, glycerol and the like. Depending on the properties of the antibody or protein composition and the carrier, it is possible to produce pharmaceutical preparations such as aerosols, dry powders and the like. In addition, the components exemplified as additives for oral preparations can also be added to the parenteral preparations.

Although the clinical dose or the frequency of administration varies depending on the objective therapeutic effect, administration method, treating period, age, body weight and the like, it is usually 10.mu.g/kg to 20 mg/kg per day and per adult.

### EXAMPLES

### Example 1: Combined Genetic and Metabolic Methods to Glycoengineer Antibodies for Conjugation

To generate the modified antibodies, the primary amino acid sequences were modified to allow for new sites of N-glycosylation to be installed. In tandem, production of the antibodies in the presence of metabolic precursors for non-natural sialic acids allows for “chemical handles” (e.g., click chemistry functional groups) to be site-specifically displayed on the antibody (or other therapeutic protein). Click ligation reactions can be used to conjugate imaging agents or drugs to the antibody or for purification of the antibody (or other therapeutic protein). Example 1 illustrates the requirement to combine multiple glycoengineering strategies as put forward in this invention to improve glycoprotein properties.

Conjugation technology can be a key determinant in bioconjugate composition, activity, and stability. Because of this, considerable interest has surrounded technologies that provide greater degree of homogeneity than that achieved through random modification of amino acid side chains. While this has been accomplished through chemical reactions using natural or unnatural amino acids, both approaches can suffer limitations and complications. For example, modification of mutated natural amino acids is complicated by the presence of multiple copies of the same residue elsewhere in the protein structure, and the use of unnatural amino acids requires bioengineered cell lines and novel expression systems. Metabolic incorporation of unnatural carbohydrate units is an alternative to existing technologies, and has become increasingly utilized in studies for the specific labeling of proteins.

The application of metabolic glycoengineering to antibody-drug conjugates (ADCs) prior to the invention herein, had shortcomings that included a maximum valency of only two per IgG antibody (i.e., each Fc chain glycan had only a single fucose when this monosaccharide was used for conjugation [Okeley, N. M., Toki, B. E., Zhang, X., Jeffrey, S. C., Burke, P. J., Alley, S. C. & Senter, P. D. Metabolic engineering of monoclonal antibody carbohydrates for antibody-drug conjugation. Bioconjug. Chem. 24, 1650-1655 (2013)]) and the high cost and low efficiency of the precursor fucose analogs. Furthermore, Fc glycans are poorly sialylated, typically <2%, preventing sialic acid from being used for ADC design; even with 100% sialylation a maximum valency of four can be achieved because almost all Fc N-glycans are biantennary. As a result the maximum antibody to drug ratio using this approach would be four, which is similar to conventional methods.

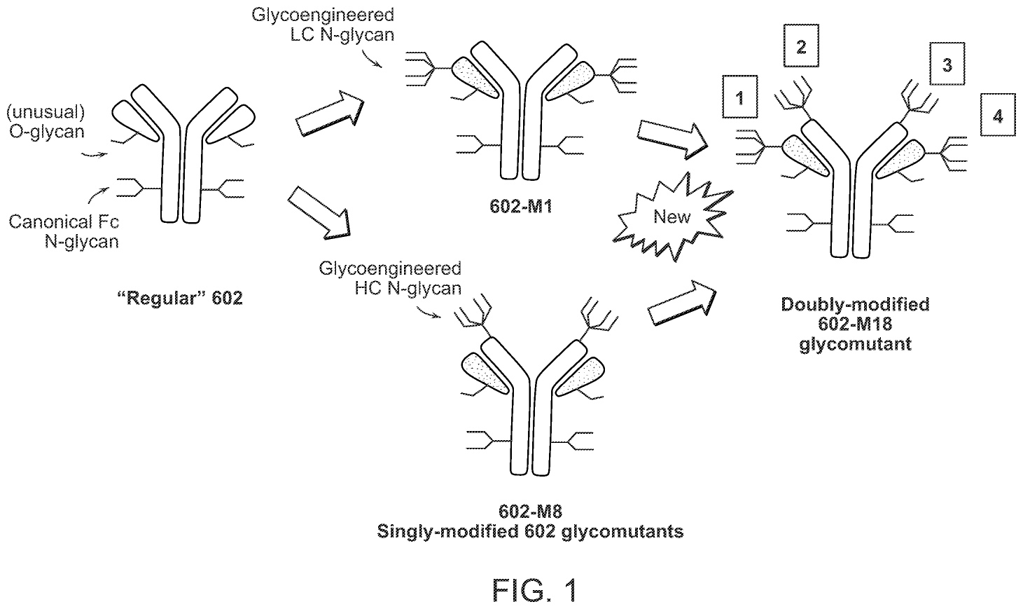

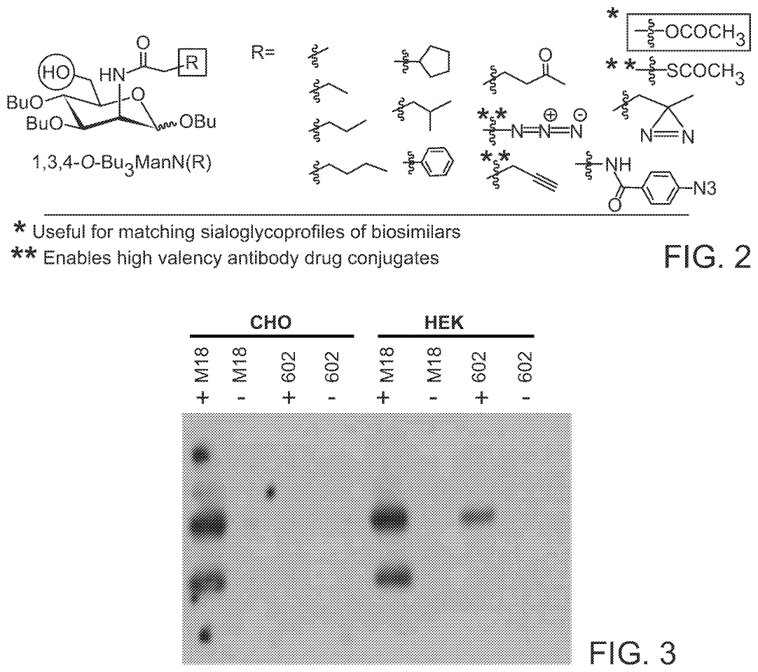

The work described here is the first example of antibody carbohydrate engineering for the purposes of making high valency, site-selectively modified ADCs by exploiting built-in, non-canonical N-glycans. The benefits and novelty of the approach described herein, includes, for example (i) the use of high flux analogs that facilitate the incorporation of non-natural, chemically-tagged monosaccharides into a therapeutic protein's glycans through cell-based metabolic glycoengineering, (ii) glycoengineered antibodies (or other therapeutic proteins) with increased valency through newly introduced N-glycan sites, and (iii) the use of genetically-engineered (e.g., GNE knock out CHO cells lines that further increase the incorporation of the non-natural sugar analogs) to mention a few.
(i) High flux analogs enable cell-based metabolic glycoengineering: A key aspect of use of sugar analogs is the “high flux” nature of butyrate-modified metabolic precursors for replacing natural sialic acid we have developed (Aich, U., Campbell, C. T., Elmouelhi, N., Weier, C. A., Sampathkumar, S. G., Choi, S. S. & Yarema, K. J. Regioisomeric SCFA attachment to hexosamines separates metabolic flux from cytotoxicity and MUCI suppression. ACS Chem. Biol. 3, 230-240 (2008), doi: 10.1021/cb7002708). These compounds enable the production of azido-modified (or otherwise modified) sialic acids on IgG antibodies (and other proteins) using living cells rather than the cumbersome and expensive chemoenzymatic production method reported by others. The position of the “built in” N-glycans (describe next, below) are designed to optimize and otherwise customize the incorporation of hexosamine analogs containing chemical tags (e.g., azide, alkyne, thiol groups and other non-natural functional groups; FIG. 2) into specific, targeted glycan structures. The basis for this technology is that the “1,3,4-O-Bu₃ManN(R) analogs enter a cell, become “deprotected” (i.e., non-specific esterases remove the ester-linked butyrate groups), and the non-natural “core” ManNAc analog enters the sialic acid biosynthetic pathway. The non-natural form of the analog replaces a proportion of the natural flux through the pathway, replacing natural sialic acids found on cell surface and secreted glycans. The analogs give rise to non-natural sialic acid on secreted IgG antibodies. These functional groups can be used for various purposes such as antibody purification, imaging agent conjugation, or drug conjugation.
(ii) Glycoengineering antibodies creates novel sites for chemical modification: To enhance glycosylation of 602, we generated a structural model of the IL-2/602 cytokine/antibody complex, and used this model to rationally design an antibody variant denoted M18. M18 represents a combination of two mutants which modify one amino acid in the heavy chain (HC) to create a new N-glycosylation site (this glycovariant is called M1) or two amino acids to create a new N-glycosylation site (M8) in the light chain (LC). Collectively, the combined mutations in the “M18” antibody introduce four new N-linked glycosylation sites in dimeric IgG. A detailed description of the design and implementation process used to create the M18 antibody is provided in Example 2, below. As illustrated in FIG. 1, the glycoengineered M18 antibody has up to 22 glycan termini, each of which can be installed with a non-natural sialic acid suitable for drug conjugation. Clearly, even if efficiency lags 100%, the potential level of incorporation exceeds conjugation levels available prior to this invention either through targeting drugs to Fc glycans or through conventional methods where typical drug to antibody ratios are ≤3-4. The number of potential sites of sialylation per dimeric IgG antibody can be increased from ˜4 to ˜22 because the M18 glycovariant has two “built-in” N-glycans per monomeric HC/LC (or 4 newly-installed glycans, overall, in the doubly-modified M18 glycomutant; FIG. 1). Even higher levels of conjugation are possible based on other classes of antibodies that have up to 6 N-glycans/chain (e.g., IgE antibodies). Note that the glycoengineering of the 602 antibody as shown in FIG. 1 is generalizable to any IgG or other class of antibody. To illustrate the benefits of using genetically modified IgG with increased N-glycan consensus sequons, pilot experiments provide proof-of-principle validation of this invention by expressing the glycoengineered M18 antibody in human HEK293 cells and wild-type Chinese hamster ovary (CHO) cells achieving a substantial gain-of-signal compared to the parent 602 antibody, based on analysis of azido-sialic acid incorporation (FIG. 3).
(iii) Increasing analog incorporation and modulating glycosylation using CHO cells with genetically modified glycosylation-modifying genes: The efficiency of analog incorporation is facilitated by complementary genetic engineering of host cells for antibody (or other recombinant) protein production that overexpress enzymes that modulate N-glycan branching (e.g. N-acetylglucosaminyltransferase), sialylation (e.g., sialyltransferases), knockout of enzymes (e.g., GNE) responsible for sialic acid metabolic flux, or knockout of enzymes that compromise glycan integrity (e.g., neuraminidases such as Neu3). The use of cells that over-express both human sialyltransferase and the GNT4/5 branching enzymes ensures increased levels of tetraantennary N-glycans. Furthermore, use of CHO GNE/RENBP-knock out cells with decreased natural flux through the sialic acid pathway promote incorporation of azido-modified sialic acids derived from 1,3,4-O-Bu₃ManNAz (or other analog containing a non-natural functional group suitable for a conjugation application). A further iteration of complementary CHO cells will express human sialyltransferases identified to selectively incorporate specific non-natural ManNAc analogs. A detailed description of the production of CHO cell lines with genetically-modified glycosylation modifying enzymes is provided in Example 3.

Applications of glycoengineered antibodies modified with non-natural sialic acids: The foundation of the technology described above, as demonstrated for the 602 antibody in its glycoengineered M18 form, can be applied to any IgG antibody, antibodies of other classes, and broadly to any therapeutic proteins. For example: antibody-drug conjugates, antibody-imaging agent conjugates, antibody purification, etc.

As described above, the introduction of genetic mutations in the primary sequence of an IgG antibody to introduce additional sites of N-glycosylation can dramatically increase the number of non-natural, “chemically-tagged” sialic acids on the antibody (FIG. 1). Each of these non-natural sialic acids can be conjugated with a fluorophore (or other imaging-enabling agent) using commercial linkers to thiols, azides, or alkynes, all of which can be introduced into N-glycans using sugar analogs (e.g., as shown in FIG. 2).

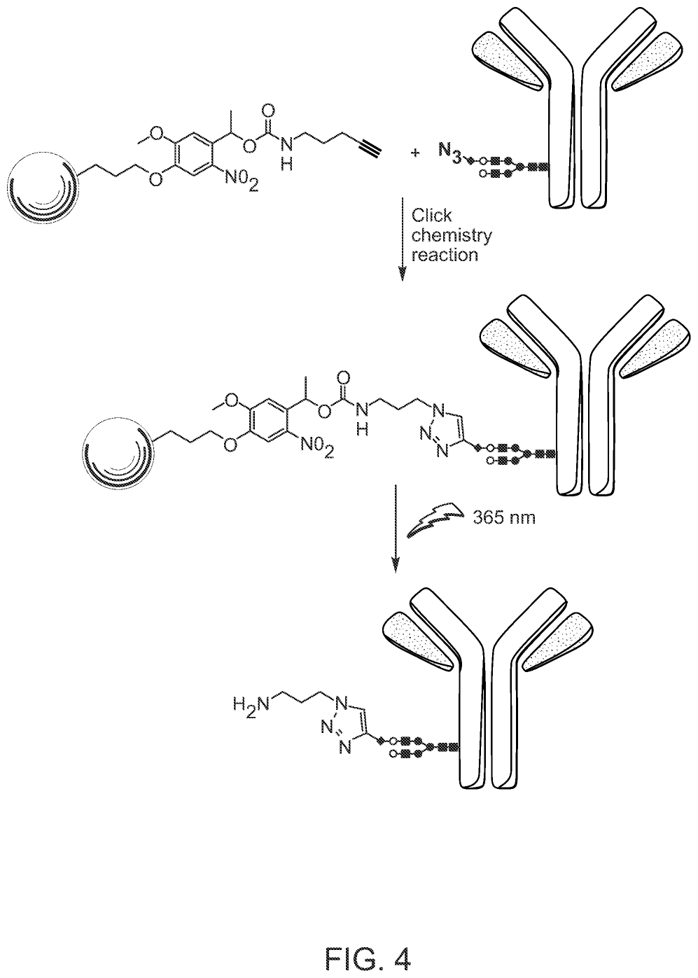

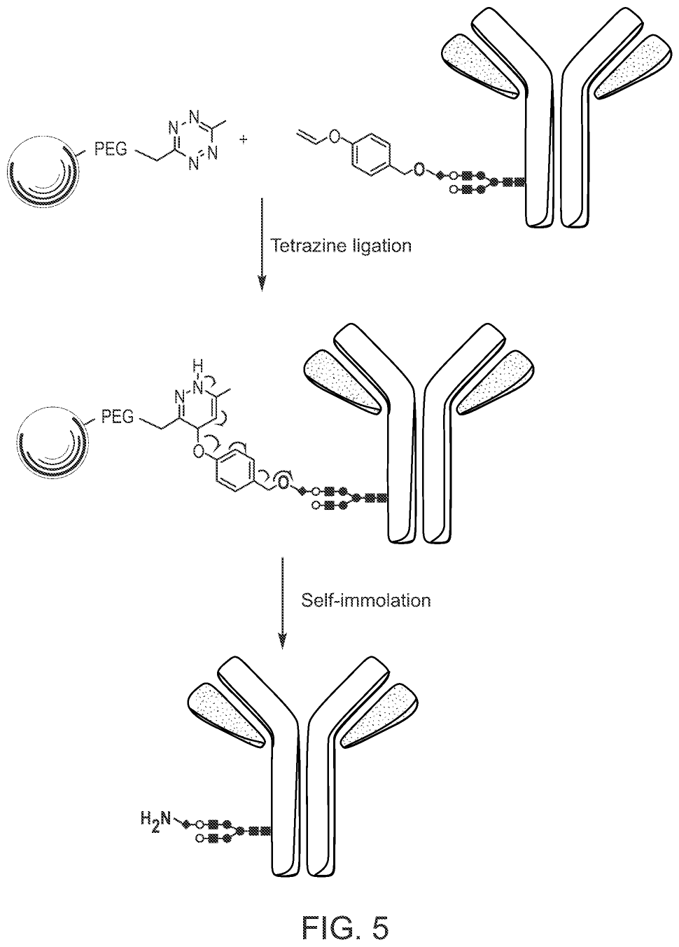

Results shown in FIG. 3, demonstrated this approach by conjugating alkyne-linked biotin to metabolically-installed azido-sialic acids (which was in turn imaged using fluorophore-tagged streptavidin). The ˜2 to 5-fold gain of signal in the M18 samples for biotin labeling provides a foundation for creating antibody-drug conjugates with enhanced valencies when the biotin used in this pilot experiment is replaced with a drug of choice. This approach can be applied using any of the other myriad commercially available “click” chemistry reagents to conjugate drugs or other compounds to the protein of interest (FIGS. 4-5). In addition to being used to design antibody-drug conjugates, this approach to creating antibody-imaging agent conjugates can be used to create research tools or as diagnostic agents.

Antibody Purification: The incorporation of “chemical tags” into the glycoengineered antibodies can be exploited to capture the antibodies on resin (etc.) and purified through controlled release (e.g., reversible thiol or “click” reactions). The state-of-the-art technology for antibody purification employs bacterial proteins A, G, and L. A limitation of this approach has been that even a trace contamination from these proteins can provoke an immune response in the host. The technology disclosed herein, tags IgG with reactive functional handles (azide or vinyl phenyl ether) that undergo chemoselective chemistry reaction with functionalized resins (alkyne containing photolabile linker or tetrazine) enabling the efficient and inexpensive selective release of the IgG using near UV (365 nm) lamp or through self-immolation (FIGS. 4 and 5). This release approach specifically liberates only bound IgG leaving behind non-specifically bound contaminants as well as contaminants from the resin.

### Example 2: Method to Glycoengineer IgG Antibodies with Non-Fc N-Glycans while Maintaining Bioactivity and Improving Pharmacokinetics

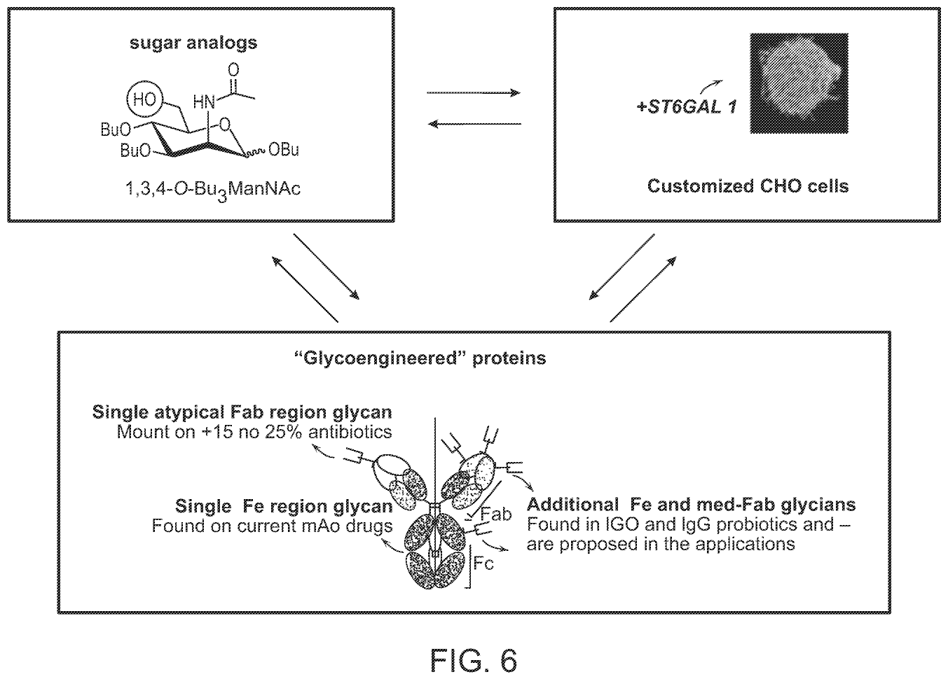

This example discloses in detail one step of our integrated design and production platform for producing glycoengineered antibodies using antibody 602 as an example of the process. Specifically the production platform consists of three distinct but interrelated components (FIG. 6) with this example focusing on the method of introducing additional, non-Fc region glycans into IgG antibodies; this is shown schematically in the lower box of the figure. This example is organized into three subsections: (a) A description of glycodesign and production process for creating glycovariants of antibody 602 is given, (b) assays demonstrating no loss of bioactivity for the glycoengineered antibodies are described, and (c) assays showing improved pharmacokinetic properties for the glycovariants are given.

### (a) Glycodesign and Production of Glycoengineered Forms of Antibody 602

Design process and criteria for “building-in” glycans into IgG antibodies, includes screening the primary DNA sequence to determine sites where the N-glycan consensus sequence can be created through the smallest number of mutations possible.

Sites for N-glycosylation can be “built in” to an IgG antibody by converting existing codons to code for the Asn-X-Ser/Thr consensus sequence, where X is any amino acid except proline (Pro). This will be done by changing a single amino to avoid steric or allosteric changes to the antibody. Online computational tools are used to predict the likelihood of glycosylation at potential sites of built-in N-glycans. Prediction tools (e.g., the NetNGlyc 1.0 Server) are used to estimate the likelihood that the built-in sequon is successfully glycosylated. Ideally (in most cases) the built-in sequon will have minimal or negligible effects on the structure of the antibody to avoid altering the antigen recognition or other functions of the antibody. In specialized cases, however, perturbations to the antibody structure maybe desired. Either scenario can be predicted using computational tools such as Rosetta or Pymoland to model the protein structure and the spatial position of the built-in glycan. The position of representative glycans built-in to the antibody are estimated using computational tools to (i) avoid steric interference with the antibody itself, (ii) avoid steric interference with antigen recognition, (iii) avoid steric interference with other proteins that form complexes with the antibody, and [iv] promote multiprotein complex formation.

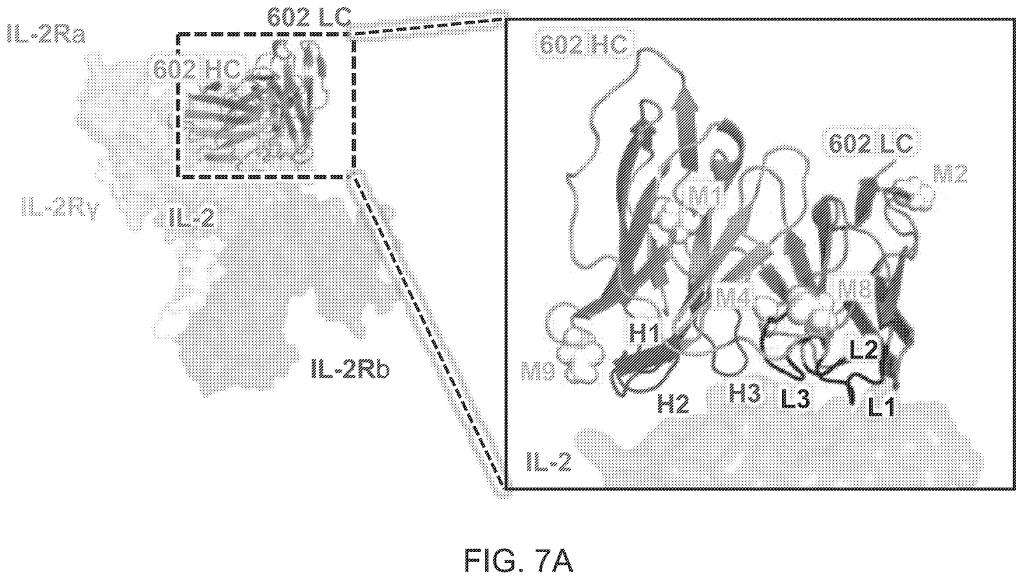

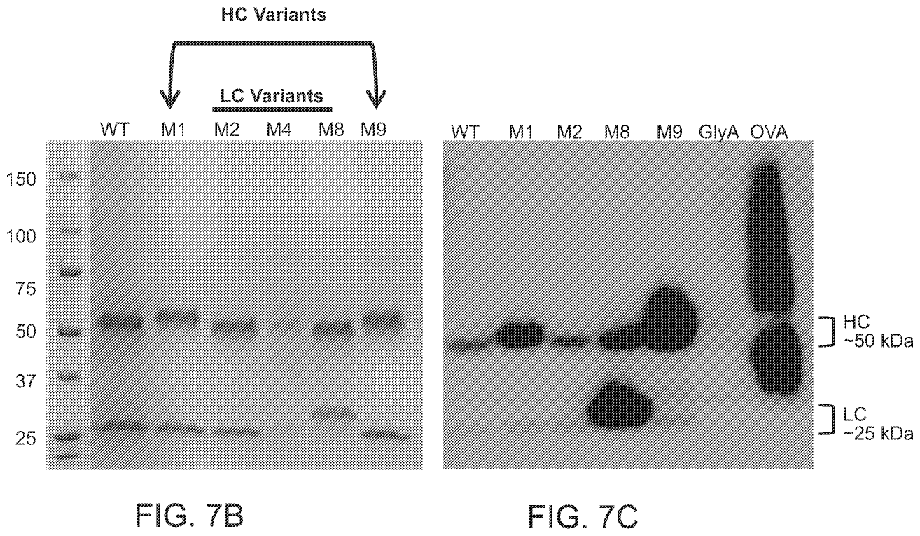

The process described was used to scan the amino acid sequence of the 602 antibody (FIG. 1). Of the numerous possibilities theoretically available, five mutants were selected (FIG. 7A). Of these five potential glycovariants, four were robustly expressed in HEK 293 cells (FIG. 7B). Amongst the four well-expressed glycovariants, three showed evidence of glycosylation based on expected shifts in the HC (M1 and M9) or LC (M8), whereas the M2 glycovariant did not have detectable glycosylation in the LC (FIG. 7B). Con-A lectin blots verified glycosylation of our engineered glycovariants (FIG. 7C). Mutants 1 and 8, one with a N-glycan built in to the HC and one built into the LC, respectively (FIG. 7A), were combined. The resulting 602 antibody variant, denoted M18, exhibited superior qualities compared to the wild type 602 antibody using the criteria described below.

Genetic modification to mutate the DNA sequence. Commercial mutagenesis kits/protocols were used to create plasmid vectors containing mutated DNA sequences coding the IgG glycovariants.

Standard expression of the mutated IgG glycovariant antibodies. In one iteration of the method, standard biomanufacturing expression systems were used to produce the mutated antibodies. Examples include HEK293 cells and Chinese hamster ovary (CHO) cell production platforms to produce therapeutic antibodies and other proteins.

Expression of the mutated IgG glycovariant antibodies in specialized host cells. Specialized host cells that can “dial in” desired glycopatterns—i.e., different glycoprofiles of N-glycans at the built-in sites of glycosylation are used.

Expression of the mutated IgG glycovariant antibodies using “high-flux” hexosamine analogs. Hexosamine analogs have various functions such as (i) complementing the specialized host cells e.g., by increasing flux for branching enzymes with 1,3,4-O-Bu₃GlcNAc or increasing flux for sialyltransferases with 1,3,4-O-Bu₃ManNAc supplementation. In another iteration, (ii) analogs with non-natural functional groups (e.g., azide, thiol, or alkynes) can be used to install “chemically-tagged” glycans into the antibodies that can be used for conjugation. Another benefit of the analogs is increased production yield of the protein, which lowers manufacturing costs.

Glycoengineered variant of the 602 anti-IL-2 antibody exhibits enhanced heavy and light chain glycosylation. expressing metabolically glycoengineered “M18” glycovariant of the 602 antibody. The M1 and M8 mutations were combined into a single, doubly-modified form of the 602 antibody (FIG. 9), treated with the 1,3,4-O-Bu₃ManNAc sugar analog. The expression of this glycoengineered antibody in HEK-293 versus CHO-K1 cells is shown by SDS-PAGE analysis in the left panel of FIG. 9 (with equal loading of all samples). A lectin blot (right) shows increased glycan levels in both the HC and LC for the double “M18” mutant, as anticipated.

### Sequences

SEQ ID NO: 1 602 and M8 Antibody Heavy Chain
Signal sequence - F5111 V_{H} - Human IgG1 C_{H}l, C_{H}2, and C_{H}3
METDTLLLWVLLLWVPGSTGDEVQLQESGPGLVAPSQSLSITCTVSGFSLTNYDISWIR
QPPGKGLEWLGVIWTGGGTNYNSGFMSRLSITKDNSKSQVFLKMNSLQTDDTAIY
YCVRQGRTPYWGQGTLVTVSAAKTTAPSVYPLAPVCGDTTGSSVTLGCLVKGYFPEP
VTLTWNSGSLSSGVHTFPAVLQSDLYTLSSSVTVTSSTWPSQSITCNVAHPASSTKVDKK
IEPRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPIER
TISKPKGSVRAPQVYVLPPPEEEMTKKQVTLTCMVTDFMPEDIYVEWTNNGKTELNYK
NTEPVLDSDGSYFMYSKLRVEKKNWVERNSYSCSVVHEGLHNHHTTKSFSRTPGK
SEQ ID NO: 2 602 and M1 Antibody Light Chain
Signal sequence - 602 V_{L} - Kappa C_{L}
MRVPAQLLGLLLLWLPGARCAGSDIQVTQSPSSLSVSLGDRVTITCKASKDIYNRLAWY
QQKPGNAPRLLISGATSLETGVPSRFSGSGSGKDYTLTITSLQTEDVATYYCQQFW
GTPYTFGGGTKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTSTSPIVKSEN
RNEC
SEQ ID NO: 3 M1 Heavy Chain
Signal sequence - F5111 V_{H} - Human IgG1 C_{H}1, C_{H}2, and C_{H}3
Highlighting denotes inserted N-linked glycosylation site
METDTLLLWVLLLWVPGSTGDEVQLQESGPGLVAPSQSLSITCTVSGFSLTNYDISWIR
QPPGKGLEWLGVIWTGGGTNYNSGFMSRLSITKDNSKSQVFLKMNSLQTDDTAIY

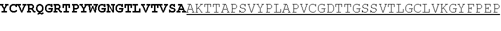

VTLTWNSGSLSSGVHTFPAVLQSDLYTLSSSVTVTSSTWPSQSITCNVAHPASSTKVDKK
IEPRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPIER
TISKPKGSVRAPQVYVLPPPEEEMTKKQVTLTCMVTDFMPEDIYVEWTNNGKTELNYK
NTEPVLDSDGSYFMYSKLRVEKKNWVERNSYSCSVVHEGLHNHHTTKSFSRTPGK
SEQ ID NO: 4 M8 Light Chain
Signal sequence - 602 V_{L} - Kappa C_{L}
Highlighting denotes inserted N-linked glycosylation site

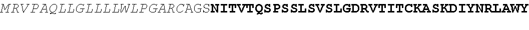

QQKPGNAPRLLISGATSLETGVPSRFSGSGSGKDYTLTITSLQTEDVATYYCQQFW
GTPYTFGGGTKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTSTSPIVKSEN
RNEC

### (b) Bioactivity of Glycoengineered Forms of Antibody 602

Binding and functional characterization studies

Cell lines: HEK 293F cells (Thermo Fisher Scientific) were cultivated in Freestyle 293 Expression Medium (Thermo Fisher Scientific) supplemented with 2 U/mL penicillin-streptomycin (Gibco). CHO-S cells (Thermo Fisher Scientific) were cultivated in Freestyle CHO Expression Medium (Thermo Fisher Scientific) supplemented with 2 U/mL penicillin streptomycin (Gibco) and 8 mM L-glutamine (Gibco). Unmodified YT-1 and IL-2Rα⁺ YT-1 human natural killer cells were cultured in RPMI complete medium (RPMI 1640 medium supplemented with 10% fetal bovine serum [FBS], 2 mM L-glutamine, 1× minimum non-essential amino acids, 1 mM sodium pyruvate, 25 mM HEPES, and 100 U/mL penicillin-streptomycin [Gibco]). All cell lines were maintained at 37° C. in a humidified atmosphere with 5% CO₂.

Biolayer interferometry binding studies: Binding studies were performed via bio-layer interferometry using an OctetRED96® biolayer interferometry instrument (Sartorius). Biotinylated human IL-2, IL-2Rα, and IL-Rb were immobilized to streptavidin (SA)-coated biosensors (Sartorius) in 0.45 μm-filtered PBSA (phosphate buffered saline [PBS] pH 7.2 containing 0.1% BSA). IL-2 and IL-2Rb were immobilized at a concentration of 50 nM for 120 s and IL-2Rα was immobilized at a concentration of 100 nM for 120 s. Less than 5 signal units (nm) was immobilized for IL-2 and each receptor subunit to minimize mass transfer effects. Once baseline measurements were collected in PBSA, binding kinetics were measured by submerging the biosensors in wells containing serial dilutions of the appropriate analytes for 300 s (association) followed by submerging the biosensor in wells containing only PBSA for 500 s (dissociation). For competitive binding studies, IL-2/antibody complexes were formed by pre-incubating a saturating concentration (600 nM) IL-2 with antibody for 30 minutes at room temperature. These complexes were then serially diluted in 600 nM hIL-2 to maintain saturating levels of cytokine. An irrelevant protein (the monoclonal antibody ipilimumab) was used as a control for nonspecific binding. Tips were regenerated in 0.1 M glycine pH 2.7. Data was processed and kinetic parameters were calculated using the Octet® Data Analysis software version 7.1 (Sartorius), assuming a 1:1 Langmuir binding model. Equilibrium binding was determined by total response measured after 295 s. Equilibrium titration curve fitting and K_{D} value determination was implemented using GraphPad Prism, assuming all binding interactions to be first order. Experiments were reproduced two times with similar results.

YT-1 cell STAT5 phosphorylation studies: Approximately 2×105 IL-2Ra− or IL-2Ra+ YT− 1 human NK cells (YT− and YT+, respectively) were plated in each well of a 96-well plate and resuspended in 20 μL of RPMI-1640 complete medium containing serial dilutions of unbound IL-2 or IL-2/antibody complexes. IL-2/antibody complexes were formed by incubating a 1:1 molar ratio of cytokine to antibody for 30 min at room temperature. Cells were stimulated for 20 min at 37° C. and immediately fixed by addition of paraformaldehyde to 1.5% and 10 min incubation at room temperature. Permeabilization of cells was achieved by resuspension in 200 μL of ice-cold 100% methanol for 30 min at 4° C. Fixed and permeabilized cells were washed twice with PBSA and incubated with a 1:50 dilution of Alexa Fluor 647-conjugated anti-phospho-STAT5 (pY694, BD Biosciences) diluted in 20 μL PBSA for 2 h at room temperature. Cells were then washed twice in PBSA and analyzed on a CytoFLEX flow cytometer (Beckman Coulter). Dose-response curves were fitted to a logistic model and half-maximal effective concentrations (EC50 values) were calculated using GraphPad Prism data analysis software after subtraction of the mean fluorescence intensity (MFI) of unstimulated cells and normalization to the maximum signal intensity. Experiments were conducted in triplicate and performed at least twice with similar results.

Results: 602 Glycovariants Retain the Function and Activity from Parent mAb.

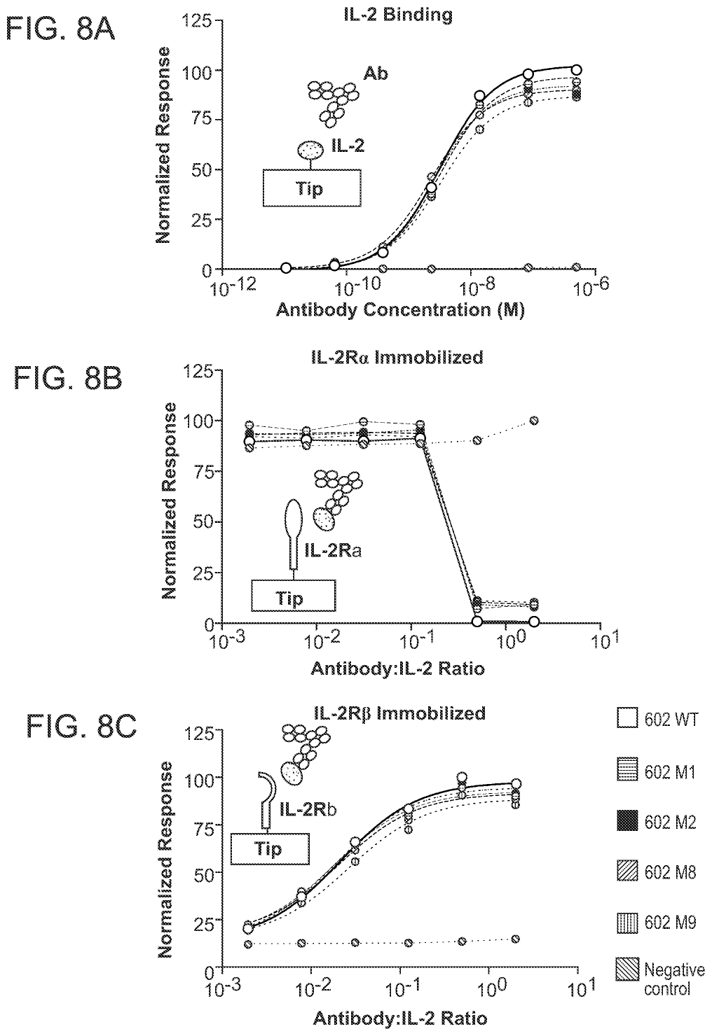

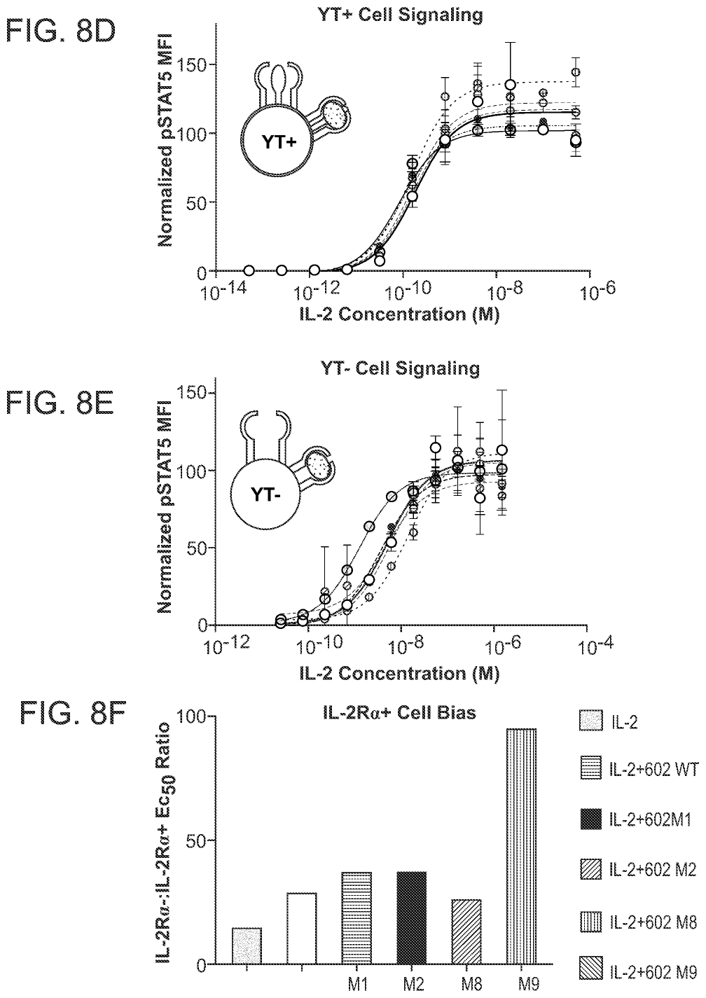

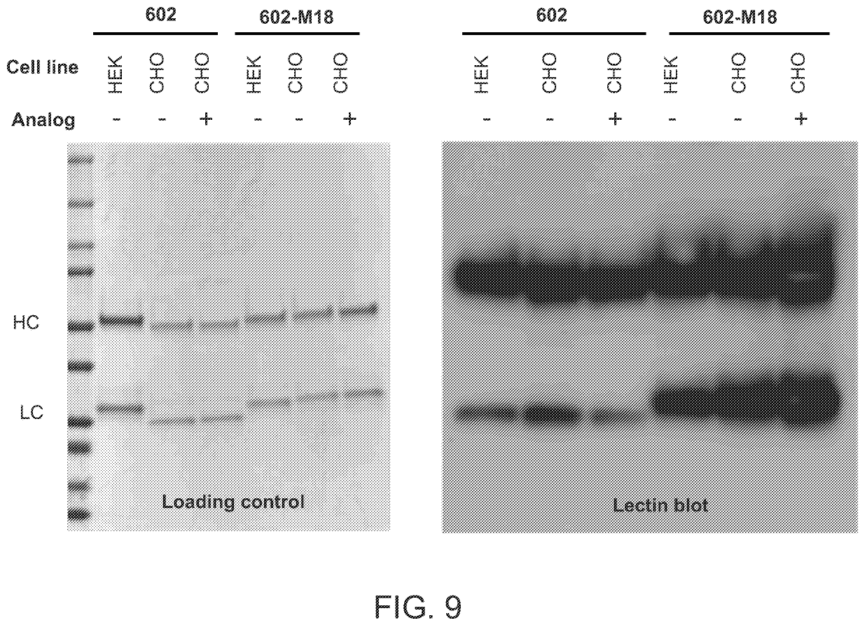

To verify that that the functional activity of 602 glycovariants mimics that of the parent mAb, we evaluated the effects of IL-2/glycovariant antibody complexes on IL-2-mediated binding and signaling. BLI studies against immobilized IL-2 demonstrated that all 602 glycovariants bound IL-2 with similar affinity compared to the parent antibody (FIG. 8A, Table 1). BLI-based competition studies between the IL-2Ra subunit and 602 antibody variants for cytokine binding demonstrated that all 3 validated glycovariants retain the inhibitory properties of the parent antibody (FIG. 8B, Table 1). Moreover, IL-2: IL-2Rb interaction was observed in the presence of the wild type 602 antibody, and this interaction was unaffected by the glycomutants, with the exception of M9, which slightly reduced the cytokine:receptor affinity (FIG. 8C, Table 1). STAT5 phosphorylation studies utilizing YT-1 human NK cell lines with and without IL-2Ra indicated that the IL-2/glycovariant complexes behave similarly to the IL-2/602 complex on both IL-2Ra− and IL-2Ra+ cells (FIGS. 8D-F, Table 1). However, M9 diverged from the other glycovariants, skewing IL-2 signaling further toward IL-2Ra+ (T_{Reg}-like) cells over IL-2Ra− (immune effector-like) cells. These results show that addition of glycans can be achieved without modifying antibody activity, although functional modulation is also possible.

TABLE 1
Fit parameters from binding and signaling pathway
studies with glycoengineered 602 antibodies.

| | kₒₙ | k_{off} | Kinetic K_{D} | Equilibrium K_{D} | YT+ EC₅₀ | YT− EC₅₀ |
|---|---|---|---|---|---|---|
| | (×10⁻⁶ M⁻¹s⁻¹) | (×10³ s⁻¹) | (nM) | (nM) | (pM) | (pM) |
| IL-2 | N.A. | N.A. | N.A. | N.A. | 84 ± 40 | 1300 ± 200 |
| 602 WT | 0.78 ± 0.00 | 1.9 ± 0.0 | 2.5 ± 0.0 | 3.2 ± 1.1 | 180 ± 150 | 5200 ± 4300 |
| 602 M1 | 0.79 ± 0.00 | 2.0 ± 0.0 | 2.5 ± 0.0 | 3.2 ± 1.3 | 160 ± 70 | 6000 ± 3300 |
| 602 M2 | 0.9 ± 0.00 | 1.9 ± 0.0 | 2.2 ± 0.0 | 2.6 ± 1.5 | 98 ± 43 | 3700 ± 2500 |
| 602 M8 | 1.0 ± 0.0 | 1.8 ± 0.0 | 1.8 ± 0.0 | 2.3 ± 0.4 | 170 ± 100 | 4500 ± 4500 |
| 602 M9 | 0.80 ± 0.01 | 2.2 ± 0.0 | 2.8 ± 0.0 | 3.3 ± 0.3 | 150 ± 60 | 14000 ± 8000 |

(c) Glycoengineered Forms of Antibody 602 have Improved Pharmacokinetic Properties

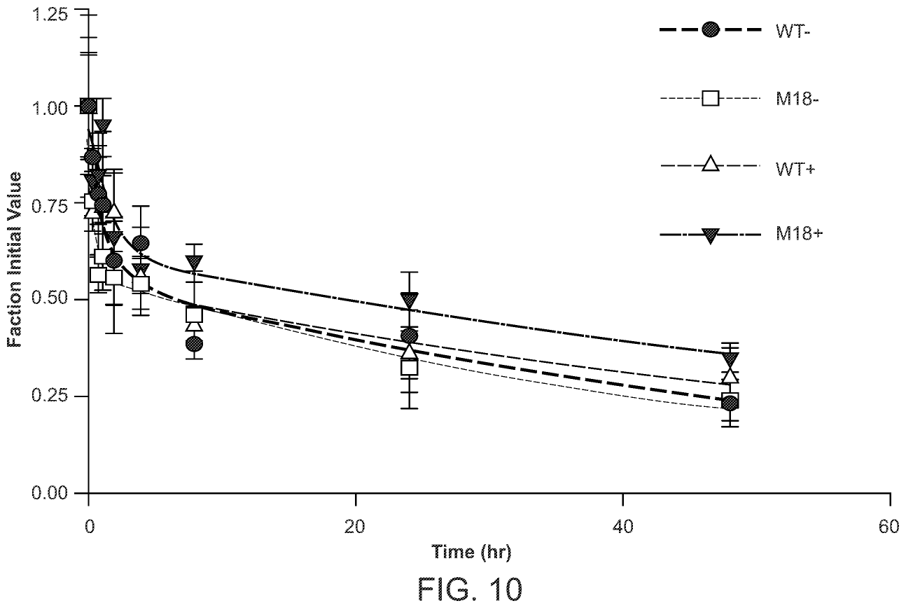

Finally, to evaluate whether the observed increase in sialylation for M18 could alter pharmacokinetic properties, we measured the serum half-life of the M18 and wild type 602 antibodies in mice. We hypothesized that the presence of sialic acid modifications would slow systemic clearance of our proteins, as was observed previously (WO 2012/020065). Mice were administered a bolus dose (10 mg/kg) of fluorescent dye-labeled antibody, and peripheral blood was collected over a 48-hour time period to monitor clearance (FIG. 10). In the absence of sialic acid analog treatment, 602 and M18 showed similar slow decay half-lives (39.5 h and 35.0 h, respectively). Addition of 1,3,4-O-Bu₃ManNAc extended the slow decay half-lives for both the 602 and M18 antibodies. Consistent with its increased levels of glycosylation due to the installed N-linked glycan sites, analog-treated M18 demonstrated a longer in vivo half-life than analog-treated 602 (60.9 h versus 49.6 h, respectively).

### Example 3: Methods to Customize CHO Cells to Improve Metabolic Glycoengineering for the Biomanufacturing of Therapeutic Proteins

Wild type CHO cells have been the “workhorse” cell line for biomanufacturing but have several glycosylation-related genes (or lack of genes) that can be genetically modified to improve the glycosylation of therapeutic proteins. In subsection (a) of Example 3 we demonstrate this concept by using CRISPR/Cas9 gene editing to delete the Neu3 sialidase gene from CHO cells; without this sialidase, glycoproteins produced in the Neu3(−) cells are expected to have higher levels of sialylation. In subsection (b) we demonstrate that this method is broadly applicable by deleting Gne and Renbp from CHO cells and show beneficial functional activity for the Gne(−) cells. Finally, in subsection (c), we demonstrate that overexpression of genes, e.g., human ST6GAL1 in conjunction with the beneficial knock outs can be achieved.

(a) Detailed Procedure for Creating Neu3 Knock Out CHO cells

### Materials and Methods

### Cell Line and Culture

A Chinese Hamster Ovary (CHO)-K1 cell line was used in all experiments. CHO-K1 cells were cultured in Kaighn's Modification of Ham's F-12 medium (F-12K) supplemented with 10% fetal bovine serum (FBS) and 2 U/mL penicillin-streptomycin (Gibco) at 37° C. and 5% CO₂

### Guide RNA Design

Single guide RNAs (sgRNAs) specific to Neu3 gene were designed using CRISPy and Benchling. Two sgRNAs targeting exon 1 and exon 2 of Neu3 gene respectively were selected to mediate CRISPR-Cas9 knockout (KO), the sequences of the two sgRNAs are given in Table 2. The criteria used when designing sgRNA were the minimized exact match and mismatched base pair, highest on-target score, combined with low off-target scores.

TABLE 2
sgRNAs designed forConstruction of Plasmids to Knock Out Neu3

| Targeted | | On-Target | Off-Target |
|---|---|---|---|
| Exon | sgRNA Sequence | Score | Score |
| 1 | 5′-CACCGAGCAGCAGTGGAATCCGGT-3′ | 66.1 | 46.0 |
| | 5′-AAACACCGGATTCCACTGCTGCTC-3′ | | |
| 2 | 5′CACCGCGTGAGAAATAGTAGGCGT-3′ | 66.3 | 32.4 |
| | 5′AAACACGCCTACTATTTCTCACGC-3′ | | |

The sequences of sgRNA 1 and sgRNA 2 are shown from 5′ to 3′ in bold and the base pair complementary to restriction site is in normal text. The sgRNA 1 sequences were used to design Plasmid 1, and the sgRNA 2 sequences were used to design Plasmid 2.

### Bacterial Transformation

Plasmid Construction and Transformation: Oligonucleotides for sgRNA were resuspended in nuclease free H₂O at a final concentration of 100 nM. They were then phosphorylated and annealed by T4 polynucleotide kinase (T4 PNK) in T4 ligation buffer in a thermocycler for 37° C. for 30 minutes, followed by 95° C. for 5 minutes. The temperature in the thermocycler was decreased from 95° C. to 25° C. at 5° C. per minute, and the samples were then taken out. The phosphorylated and annealed oligonucleotides were diluted to 1:200 by adding 1 μL of oligo mix to 100 μL of room temperature double distilled water (ddH₂O) and ligated to a no-insert pSpCas9(BB)-2A-GFP (PX458)-only negative control in a set ligation reaction containing FastDigest BbsI. Incubation of the ligation mixture follows a cycle consisting of 37° C. for 5 minutes and 21° C. for 5 minutes for six repetitions. Uncut PX458 plasmid was employed as the positive control and demonstrated whether transformation occurred or not; additionally, PX458 digested with BbsI was used as the negative control as no colonies should be present without ligation. To achieve transformation, 2 μL of ligated plasmid was added to 20 μL of ice-cold chemical competent was E. coli strain Stbl1 cells. After incubating for 10 minutes, heat shock at 42° C. was conducted for 30 seconds followed by putting the samples on ice for 2 minutes, with the result of enabling the entry of the plasmid into the Stbl1 cells. SOC media (100 μL) was added into the tube containing Stbl1 cells and either 20 μL or 80 μL of mixture was further seeded on two LB plates containing 100 μg/mL ampicillin separately. After overnight incubation at 37° C., 4 colonies with transformed plasmid 1 or 2 were picked using sterile pipet tip and incubated in 5 mL of LB medium with 100 μg/mL ampicillin and shaken at 37° C. overnight.

Plasmid Isolation: The QIAprep Spin Miniprep Kit purchased from QIAGEN was used to isolate the plasmids from the bacteria. For each sample, 1.5 mL of bacterial overnight culture were pelleted into a 2 mL microcentrifuge tube and centrifuged at 10,000 rpm for 3 minutes. This step was repeated in order to acquire more bacterial cells. The remaining bacterial overnight culture was saved for glycerol stock preparation. The pelleted bacterial cells were resuspended in 250 μL Buffer P1, then 250 μL of Buffer P2 was added and mixed thoroughly until the solution had turned from blue to clear. After five minutes, 350 μL of Buffer N3 was added and mixed thoroughly. Then the microcentrifuge tubes were centrifuged at 13,000 rpm for 10 minutes. After centrifuging, 800 μL of the supernatant from previous step was applied to the appropriately labeled QIAprep 2.0 columns via pipetting. Vacuum manifold processing was used to draw the solution through the columns. For washing, 500 μL of Buffer PB was first added to the columns and the solution was drawn through via the vacuum manifold, then 750 μL of Buffer PE was similarly added and drawn through via the vacuum manifold. The columns were centrifuged at 13,000 rpm for 1 minute to remove the residual wash buffer. The columns were then placed in clean 1.5 mL microcentrifuge tubes and 50 μL of Buffer EB was added to elute the DNA. After 1 minute, the tubes were centrifuged at 13,000 rpm for 1 minute and the plasmids were extracted. The concentrations of the plasmids were then measured using a Nanodrop. From the remaining, unused bacterial overnight cultures, 750 μL of cell culture and 750 μL of glycerol were mixed to form 50% glycerol stocks, before being stored for use in the future.

Plasmid Sequencing: For verification purposes, the isolated plasmids were sequenced from the U6 promoter using the“U6-Fwd primer”: 5′-GAGGGCCTATTTCCCATGATTCC-3′.

### CHO-K1 Cell Transfection

CHO-K1 Cell Seeding: The media in the flask was aspirated, then 5 mL of PBS was added to wash and aspirated. Trypsin was added at a volume of 3 mL and the flask was incubated for 5 minutes. After the incubation, the trypsin/cell solution was pipetted to a 15 mL centrifuge tube and 7 mL of media were added into the flask and pipetted to the 15 mL centrifuge tube. The tube was centrifuged for 3 minutes at 1,500 rpm. After centrifuging, the supernatant was aspirated carefully. The media was added at a volume of 10 mL to resuspend the cells. The resuspended solution was added at a volume of 20 μL to 200 mL of PBS. After the cell count was determined, 20,000 cells were seeded to each well in a 6-well plate and extra media was added to reach a total volume of 2 mL.

Plasmid Purification from E. coli: Liquid bacteria cultures were prepared from the glycerol stocks before experimentation and the plasmids were purified from the bacteria using the QIAprep Spin Miniprep Kit purchased from QIAGEN, similarly to the plasmid isolation step previously described in plasmid isolation part above.

Transfection: For each well of cells on the 6-well plate, 2.5 μL of Lipofectamine 2000 was diluted into 250 μL of Opti-MEM I Reduced Serum Medium without serum, added to the individual wells, and incubated for 5 minutes at room temperature. 2,500 ng of the sequence verified PX458 DNA Plasmid 1 or plasmid 2 or 2,500 ng of Plasmid 1 and 2,500 ng of plasmid 2 was diluted into 250 μL of Opti-MEM I Reduced Serum Medium without serum. After mixing the 250 μL of diluted DNA solution and 250 μL of diluted Lipofectamine 2000 solution, the mixture was incubated for 20 minutes at room temperature to allow the formation of DNA-Lipofectamine 2000 complexes. The growth media was removed from the wells and replaced with 2 mL F-12K growth media without FBS and antibiotics. In each well containing cells, 500 μL of DNA-Lipofectamine 2000 complexes was added dropwise and mixed gently by rocking the plate back and forth. After incubating for 24 hours, fresh F-12K media supplemented with 10% FBS and 2 U/mL penicillin-streptomycin (Gibco).

Cell Sorting: After another 24 hours of incubation, CHO-K1 cells were harvested. GFP positive and propidium iodide (PI) negative CHO-K1 cells were sorted by Fluorescence-Activated Cell Sorting (FACS). Single cells were seeded into the wells of a 96-well plate respectively.

### Validation of Gene Editing

Primer Design: Primers for amplifying a region of genomic DNA with a size of 600 to 1200 base pairs (sgRNA 1 or sgRNA 2), containing the site where sgRNA edited, were designed respectively using online Primer Quest Tool published by Integrated DNA Technologies (IDT) (Table 3).

TABLE 3
Primers used to amplify a region of genomic DNA near the Cas9 cut site of
each gene target

| Plasmid | | | | T_{M} | |
|---|---|---|---|---|---|
| sgRNAs | Primers | Sequence | Length | (° C.) | GC% |
| sgRNA | Forward | 5′-TATCAGCCTGCTCCCTCAA-3′ | 948 | 63 | 52.6 |
| 1 | Reverse | 5′-CTGATGACCTGGGTTTGATCTC-3′ | | 62 | 50 |
| sgRNA | Forward | 5′-AGCCGTGTGTTCCTGTTT-3′ | 619 | 62 | 50 |
| 2 | Reverse | 5′-GAGTAGTGGGAGCAGCTTTAC-3′ | | 62 | 52.4 |

Cell Stock Preparation: From each of three 96-well plates, 6 samples containing cells with ˜100% confluency were selected and seeded into the wells of a 6-well plate respectively. Subsequently, the three 6-well plates containing edited single-cell clones for each CRISPR/Cas9 condition (Plasmid 1, Plasmid 2, Plasmid 1+2) were examined under the light microscope for confluence. From each plate, three of the most confluent colonies were chosen for further analysis. Three quarters of the cell mixture was added to an Eppendorf tube for freezing in 10% DMSO solution. The tubes were inverted gently a few times to mix, and the cells were placed into the −80° C. freezer before being transferred to liquid nitrogen storage. The remaining volume was added to a different Eppendorf tube for mutation detection. The cells were spun down in a microcentrifuge at 2,000 rpm for 3 minutes, and the media and trypsin were aspirated without disturbing the cell pellets.

Genomic DNA Isolation from CHO-K1 Cells: The samples that were to be used for mutation detection were washed two times with 300 μL PBS by adding the PBS to the Eppendorf tubes, resuspending, and spinning down in the microcentrifuge. After the second wash, the pelleted cells were lysed by adding 50 μL Quick Extract DNA Extraction Solution. The cell lysates were transferred to PCR tubes, vortexed, and heated in a thermocycler at 65° C. for 10 minutes followed by 98° C. for 5 minutes. After heating, 100 μL nuclease-free water was added to each sample to dilute the genomic DNA, and the samples were vortexed.

Genomic DNA Amplification by POR: The lyophilized sgRNA primers (IDT) were resuspended in the appropriate volume of nuclease-free water to create a 100 μM solution. From these 100 μM stocks, 9 μM dilutions were created in separate Eppendorf tubes. The PCR reactions were set up using the appropriate volumes of genomic DNA (˜40 ng), forward primer (final conc. 300 nM), reverse primer (final conc. 300 nM), OneTaq Hot Start 2X Master Mix containing the polymerase, and nuclease-free water. The total reaction volume was 30 μL. For the samples with Plasmid 1+2, separate reactions were set up with sgRNA 1 primers and with sgRNA 2 primers. In the thermocycler, the samples were first heated to 95° C. for 5 minutes. The samples then underwent 40 cycles of the following steps: denaturing at 95° C. for 30 seconds, annealing at 57.5° C. for 30 seconds, and extending at 72° C. for 30 seconds. The annealing temperature of 57.5° C. was chosen because it is ˜5° C. below the Tₘ of the primers. Once the 40 cycles were completed, the samples were extended again at 72° C. for 30 seconds. The samples were stored at 4° C. until they were analyzed by agarose gel electrophoresis, sequenced, or used for gene editing.

Agarose Gel Electrophoresis: An agarose gel (1% w/v) was made at a volume of 50 mL by adding 0.5 g of agarose to an Erlenmeyer flask containing 50 mL of 1×Tris Acetate EDTA (TAE) buffer. The flask was microwaved for 90 seconds to allow the dissociation of agarose in the solution. After letting the solution cool at room temperature for 10 minutes, 2 μL of ethidium bromide was added to reach a final concentration of 0.4 μg/mL. The gel was poured from the flask to the set gasket. The gel solidified, and a sufficient amount of TAE buffer was added so that the gel was completely submerged. DNA loading dye was added at a volume of 2 μL into 8 μL of each PCR product and 1 μL of DNA loading dye was added into 4 μL of the 1 kb DNA ladder. All of the samples were loaded into the wells and gel electrophoresis was conducted at 120 volts for 30 minutes.

POR Product Sequencing: PCR purification was carried out on three colony samples of each plasmid with their respective primers. Six colony samples of the combined plasmids, three with primers for sgRNA 1 and three with sgRNA2 were also purified. A commercially available preparation kit from QIAGEN was used (QIaquick PCR Purification Kit, 28106) with modified protocol for low DNA concentrations. Buffer PB (110 μL) was added to each sample (22 μL) at five a five-volume ratio. Each sample was processing using a vacuum manifold, washed with 750 μL Buffer PE, vacuumed, and then centrifuged at 17,900 g for one minute. DNA was eluted by adding 25 μL of distilled water, waiting one minute, and then centrifuging at 17,900 g for one minute. Purified samples were analyzed using a Nanodrop microvolume spectrophotometer for DNA retention by loading 1 μl of each prepared sample. Primers from each plasmid line were selected based on proximity to the target NEU3 sequence. For both plasmids, the forward primer was selected. Sanger sequencing was performed on the 12 purified samples by the Johns Hopkins Genetic Resources Core Facility using an Applied Biosystems 3730xl DNA Analyzer.

### Results

### Bacterial Transformation

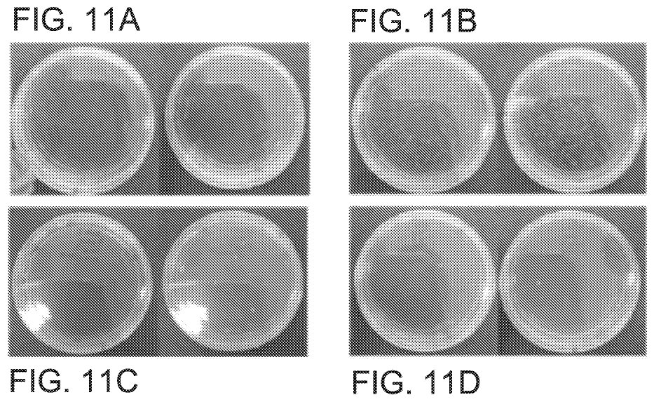

Plasmid Construction and Transformation: As shown in FIGS. 11A and 11B, Stbl1 E. coli transformed with unligated PX458 could not grow on LB plates containing ampicillin, while others transformed with cut PX458 could robustly grow under the same condition. The morphology of the small colonies was similar, with white and round features. In terms of E. coli containing sgRNA 1 or sgRNA 2 constructs, due to the difference in the amount of cell mixture added (20 μL and 80 μL), the number of colonies present on the LB plates was altered. As shown in FIGS. 11C and 11D, the LB plates seeded with 20 μL of cell mixtures (left figures) only displayed one or two colonies, while the other plates seeded with 80 μL of cell mixtures exhibited an increased number of colonies. There were 8 colonies containing sgRNA 1 inserted plasmids and 4 colonies containing sgRNA 2 inserted plasmids.

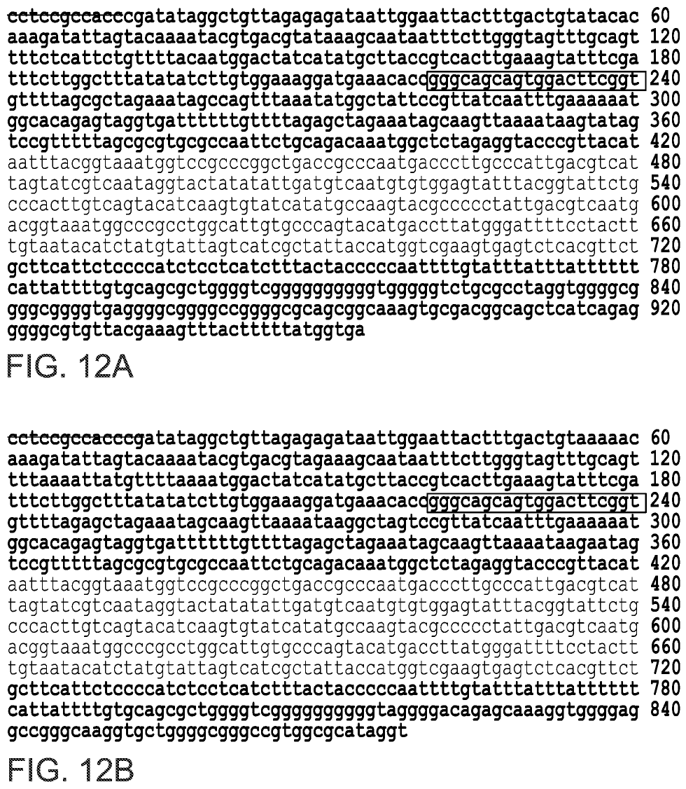

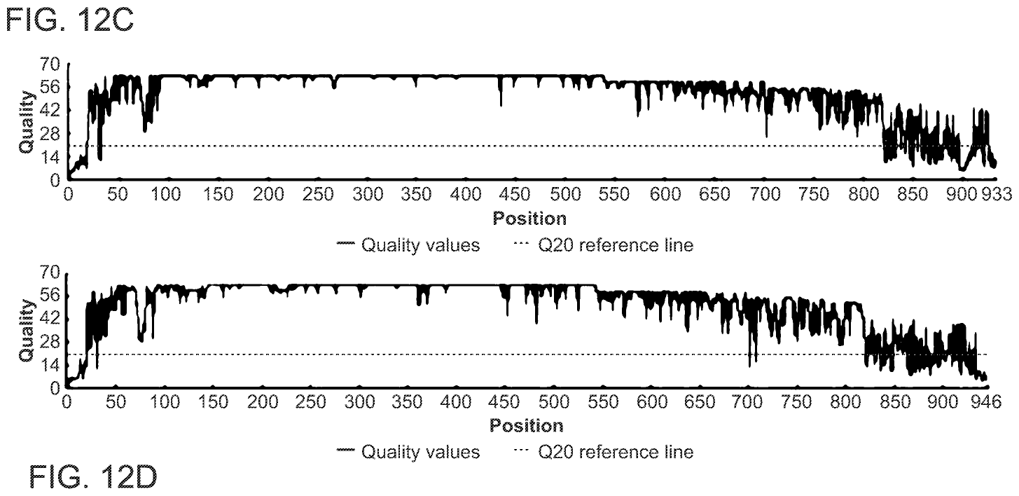

Plasmid Sequencing: The representative sequencing results of Plasmid 1 and 2 are shown in FIGS. 12A and 12B respectively. The highlighted regions represent the designed sgRNA sequences, indicating the successful insertion of sgRNA into the plasmids. Furthermore, the quality score curves were generally flat, and sat above the reference line as indicated in FIGS. 12C and 12D.

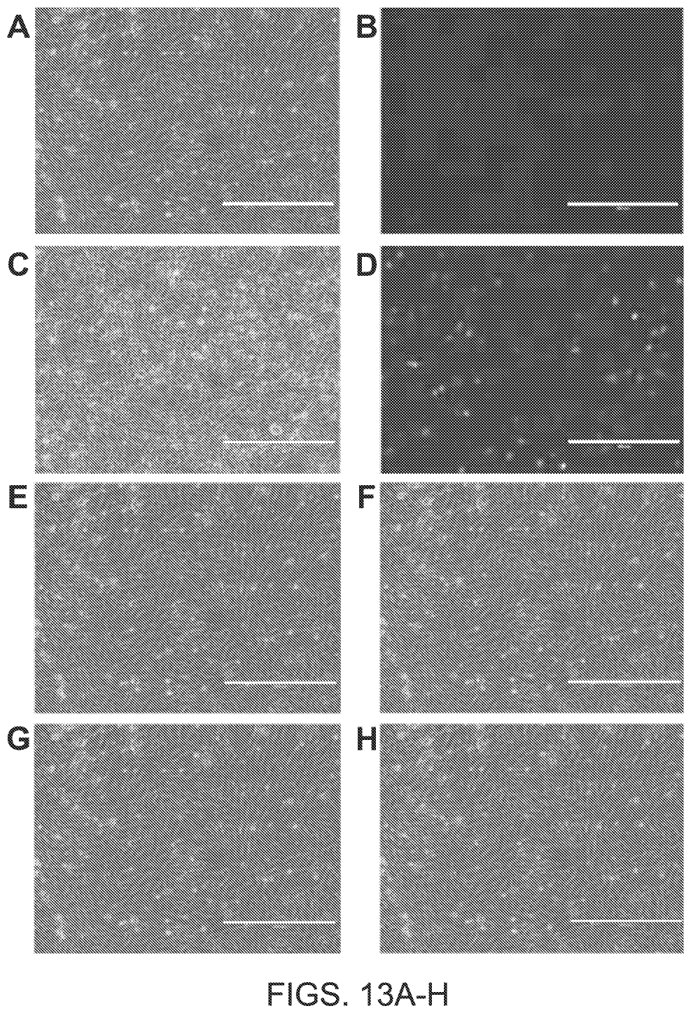

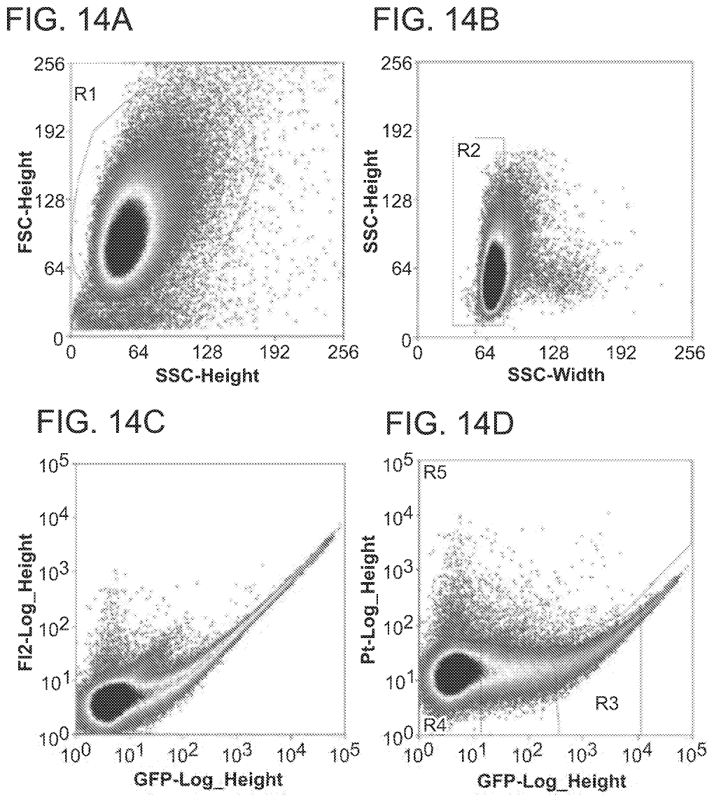

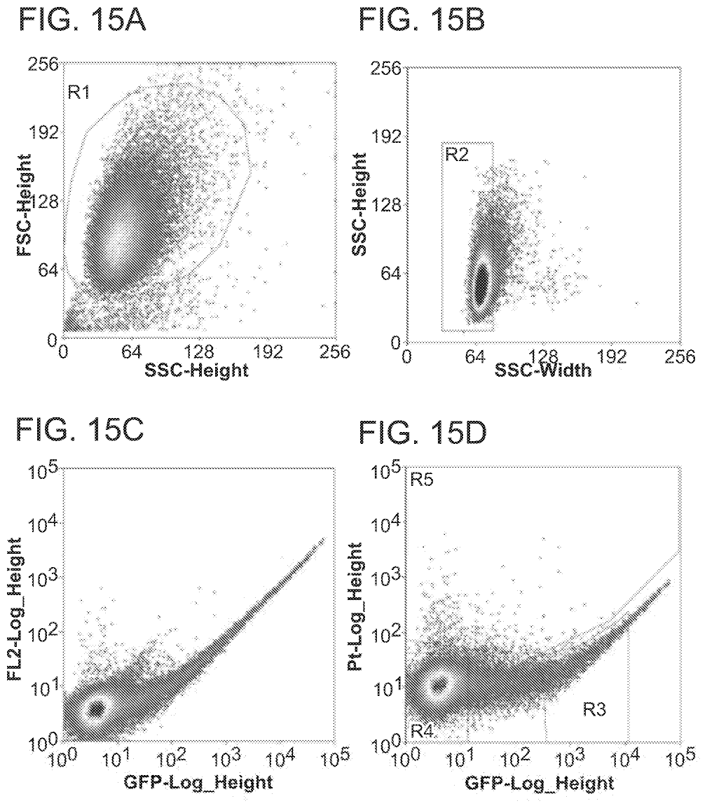

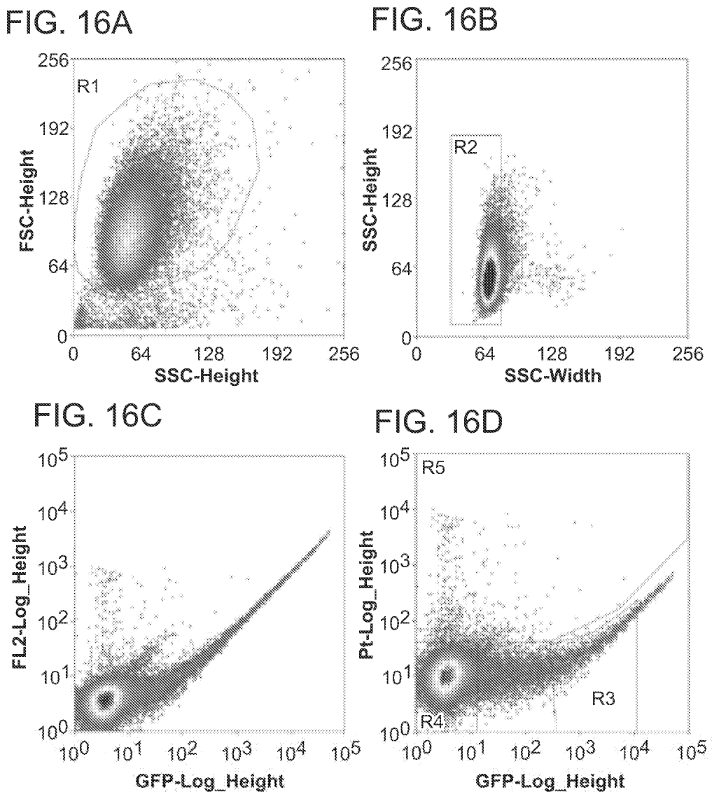

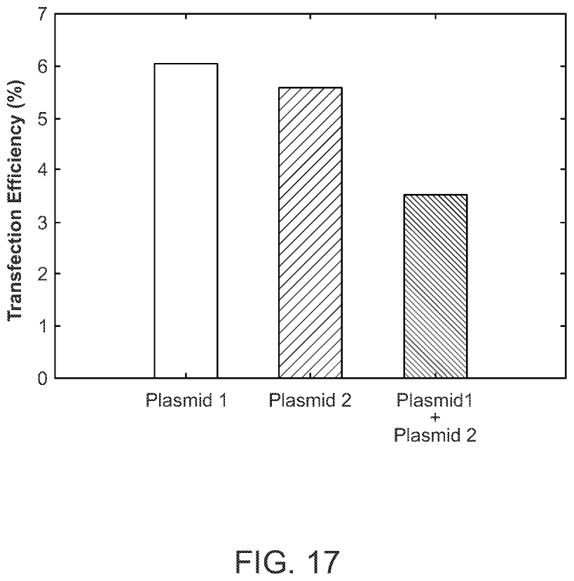

FACS Results: Qualitatively, the control did not exhibit fluorescence, which demonstrated that transfection had not occurred in those CHO-K1 cells (FIGS. 13A-13H). The Plasmid 1, Plasmid 2, and combination of Plasmid 1 and Plasmid 2 conditions displayed GFP fluorescence, indicating that some of the cells had been transfected. FACS data for the transfection of Plasmid 1, Plasmid 2, and the combination of Plasmid 1 and Plasmid 2 into CHO-K1 cells was analyzed via gating, as seen in FIGS. 14A-14D, FIGS. 15A-15D, FIGS. 16A-16D and FIG. 17, respectively. The first gate was based on cell morphology. The data was then gated in order to select single cells, instead of aggregates of cells. In order to select for transfected cells, the data was gated for GFP positive cells and propidium iodide negative cells, with propidium iodide acting as a cell viability stain that indicates dead cells. The transfection efficiency was then calculated by dividing the GFP positive, propidium iodide negative cells (gated by region R3) by the total number of single cells and multiplying by 100 to find the percentage. Region R4 gated the cells that were viable and not transfected. The region between R3 and R4 was not included when calculating the transfection efficiency, due to the ambiguity of whether those cells were transfected or not. Plasmid 1 exhibited a similar transfection efficiency to Plasmid 2, and the combination of Plasmid 1 and Plasmid 2 had a slightly lower transfection efficiency in comparison to the individually transfected plasmids (FIG. 17).

### Validation of Gene Editing

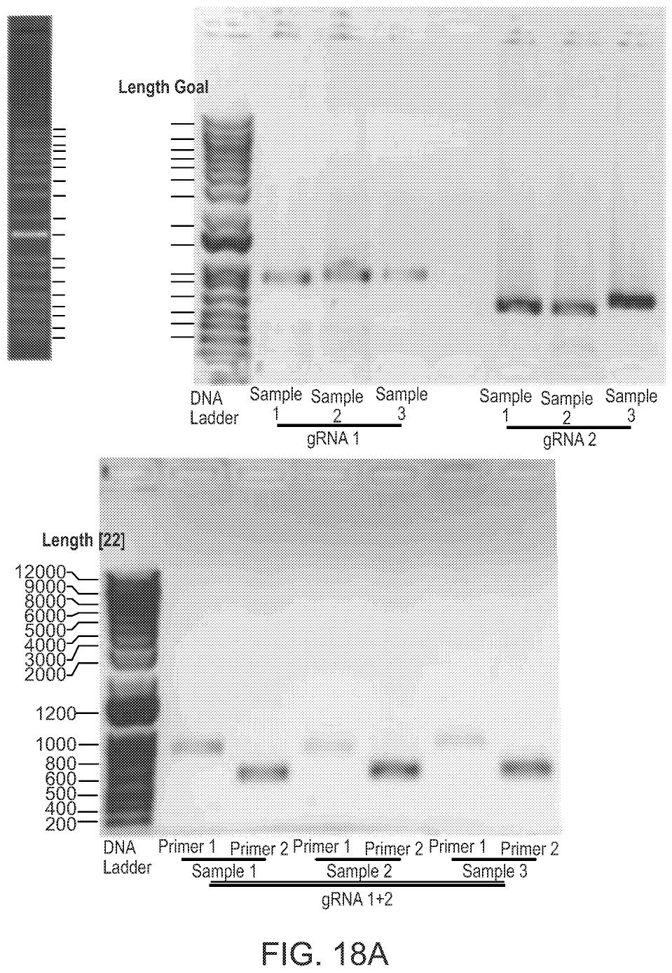

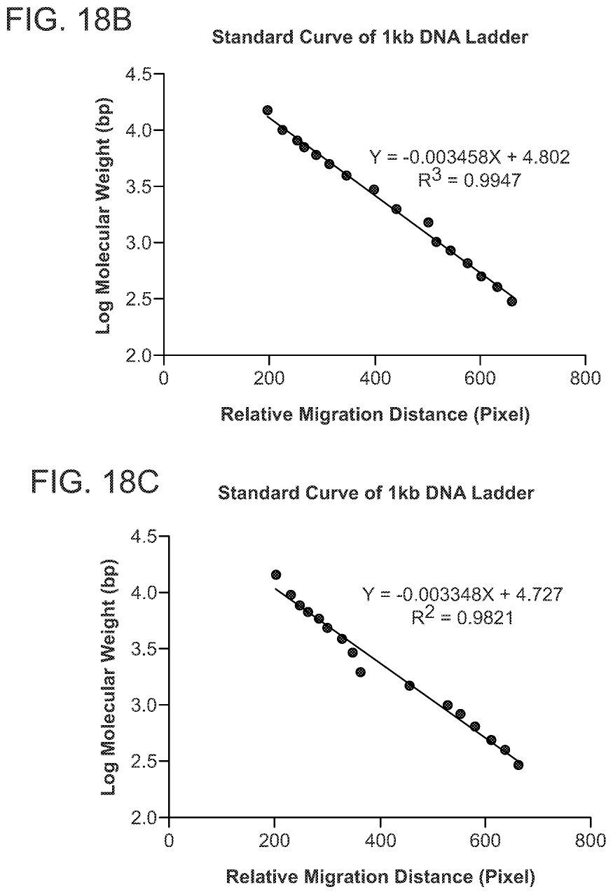

POR Amplification: The left part of FIG. 18A shows a visualization of the PCR products, with the bands representing the genome DNA amplified by either sgRNA 1 primer or sgRNA 2 primer, i.e. contains the sites where sgRNAs should target. The log form of molecular weight was a function of relative migration distance. Based on the equation shown in FIGS. 18B and 18C, the size of each PCR product could be calculated. The length of PCR products with the modification of sgRNA 1 directed CRISPR-Cas9 system was 1140.250 bp, 1040.878 bp, and 1056.818 bp, respectively, while that of the PCR products with the modification on gRNA 2 targeted region was approximately 640.030 bp, 630.377 bp, and 711.847 bp, respectively. According to the calibration curve of the 1 kb DNA ladder indicated in FIG. 18C, the sizes of DNA fragments amplified using sgRNA 1 primer in samples 1, 2, and 3 were 935.829 bp, 912.433 bp, and 900.935 bp, respectively, and the DNA fragments amplified using sgRNA 2 primer in samples 1, 2, and 3 were 669.884 bp, 651.447 bp, and 649.830 bp, respectively. The presence of bands provided evidence that the designed primers functioned correctly.

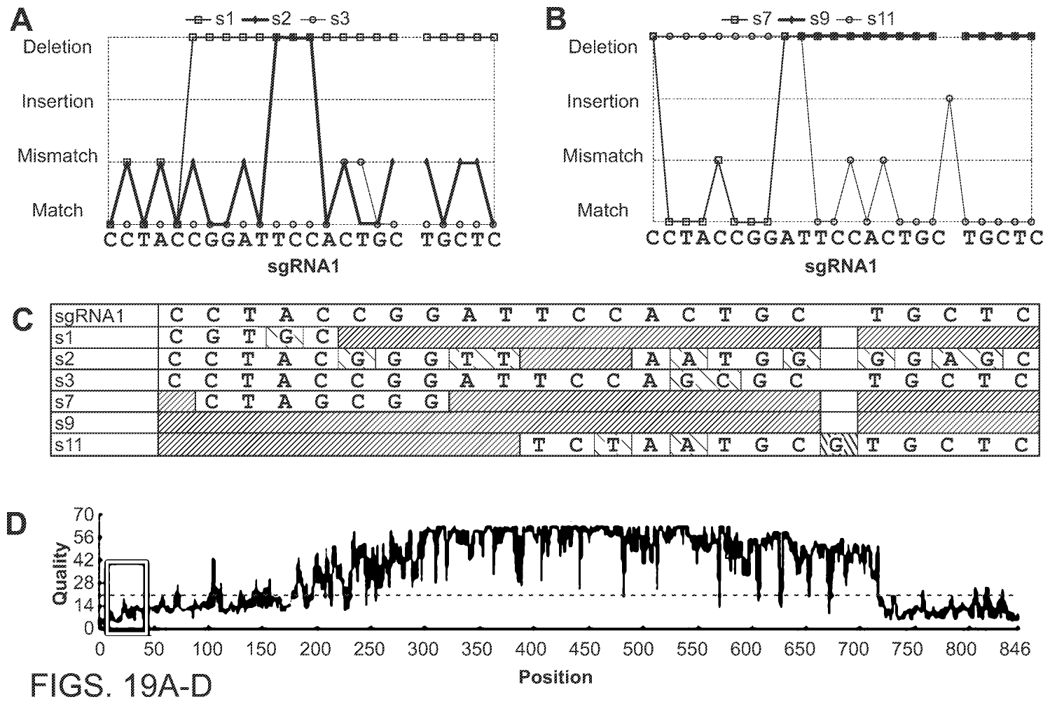

POR Product Sequencing: The purified PCR products were prepared for Sanger sequencing to verify any genomic changes. Primers for each sgRNA target were chosen for their proximity to the target strand. The forward primers previously designed were used for both sgRNA targets. The MUSCLE algorithm was used for alignments. FIGS. 19A-19D shows the effects of CRISPR/Cas9 editing on sgRNA 1. The specific point mutations at the target site are shown by FIGS. 19A-19C while FIG. 19D is a representative quality score from the Sanger sequencing. Samples for Plasmid 1 are given by s1-s3 while the combined Plasmid 1+2 samples are represented by s7, s9, and s11. Consistent knockout regions along the sgRNA target were observed, with the exception of s3 (Plasmid 1, colony 3). One sample also produced a single point insertion (s11). The overall sequencing quality for the region of interest was relatively low, possibly accounting for the point mismatch mutations.

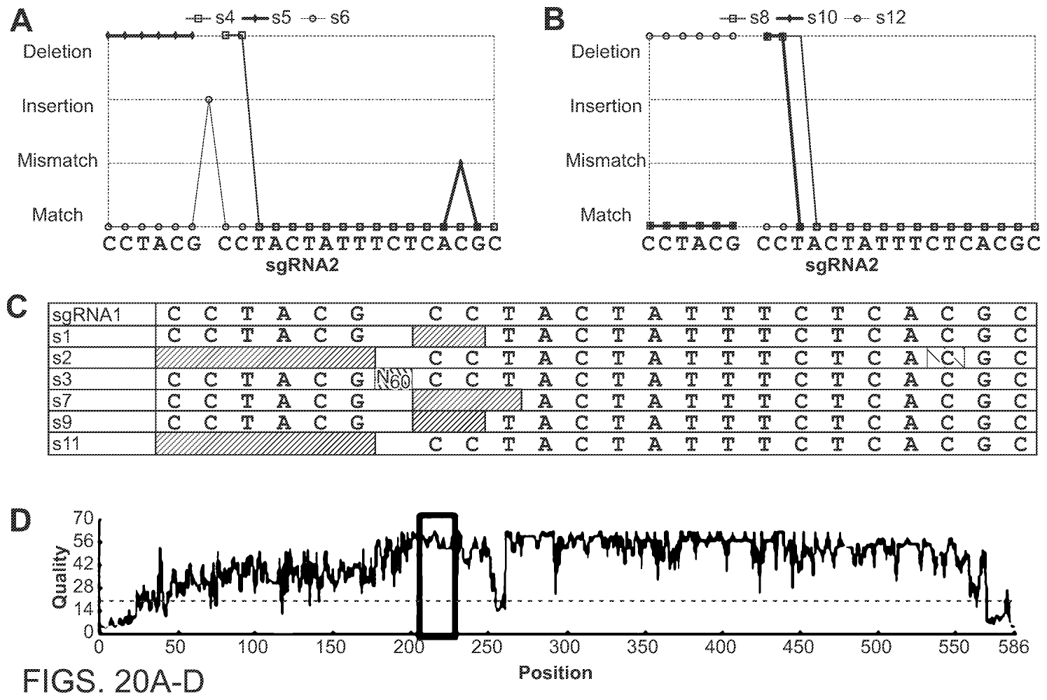

The genome editing efficacy of Plasmid 2 at the target region is given by FIGS. 20A-20D. FIGS. 20A-20C shows the point mutations for isolated plasmid 2 (s4-s6) and combined plasmids (s8, s10, s12). The quality of Sanger sequencing is given by FIG. 20D, showing the quality of s5 specifically.

### (b) Knock out of Gne and Renbp in CHO Cells for Control of Metabolic Flux

### Rationale

Control of metabolic flux required for glycosylation is complex and deliberate manipulation of one facet of the system can counteract complementary endpoints. For example, N-glycan branching to convert prevalent biantennary type N-glycans to larger tri- and tetraantennary glycans that provide therapeutic proteins with improved pharmacokinetic properties requires two components. The first is the presence of GNT4/5 (human) or Gnt4/5 (rodent) enzymes coded by the MGAT4/5 (or Mgat4/5) genes. GNT/Gnt4 installs the GlcNAc residue needed to initiate the third branch of triantennary N-glycans and GNT/Gnt5 installs the GlcNAc residue needed for the fourth branch of tetraantennary N-glycans.

A second requirement for GNT4/5 activity is higher than naturally-occurring levels of UDP-GlcNAc, which is the substrate for these enzymes. Cellular levels of UDP-GlcNAc typically are 0.1 to 1.0 mM, but the Kₘ for GNT4 is ˜5 mM and for GNT5 is ˜11 mM, meaning that these enzymes are minimally active in cells—including CHO cells used in biomanufacturing—under normal conditions. One way to overcome this pitfall is through supplementation of the culture medium with GlcNAc, however this approach is not practical for biomanufacturing because of the high concentrations (e.g., 10 to 50 mM) of this sugar needed to increase intracellular levels of UDP-GlcNAc. Problems with using such high levels range from scientific (e.g., osmotic shock that decreases cell viability) to the economic (GlcNAc is a relatively expensive monosaccharide, e.g., $20/gram from Sigma-Aldrich).

To overcome these problems, high-flux GlcNAc analogs (e.g. 1,3,4-O-Bu₃GlcNAc and Bu₄GalNAc) can be used as part of our biomanufacturing platform (FIG. 6) to increase flux through the hexosamine biosynthetic pathway, and increase the activity of the GNT4/5 branching enzymes. A pitfall of this strategy, however, is that UDP-GlcNAc can be converted to ManNAc by GNE/Gne and GlcNAc can be converted to ManNAc, by RENBP/Renbp. As a result, the use of a supplement such as 1,3,4-O-Bu₃GlcNAc to increase N-glycan branching increases levels of ManNAc in a cell leads to high levels of this sugar, which out competes exogenously added non-natural metabolic precursors for sialic acid such as 1,3,4-O-ManNAz, resulting in 2 to 20% replacement of natural sialic acid with non-natural forms of this sugar. As a consequence, the incorporation of azido-groups for purposes such as creation of antibody-drug conjugates described in Example 1 is suboptimal. This pitfall can be ameliorated by genetically eliminating the routes for endogenous ManNAc flux into the sialic acid pathway non-natural thereby facilitating increased metabolic incorporation of non-naturally modified forms of sialic acid into the glycans of recombinant proteins. We anticipate benefits even in the absence of “high flux” UDP-GlcNAc-promoting metabolites such as 1,3,4-O-Bu₃GlcNAc because even endogenous UDP-GlcNAc levels are sufficient to introduce competing flux into the sialic acid biosynthetic pathway. To overcome this problem, we created gene edited Renbp(−) and Gne(−) CHO cells.

### Methods

The procedure described in detail in subsection (a) above for Neu3 was followed by using the appropriate sgRNAs (Table 4) and sequencing primers (Table 5) for Renbp and Gne.

TABLE 4
Sequences of DNA oligos (sense and anti-sense) cloned into the
pSpCas9(BB)-2A-GFP (PX258) plasmid. Base pairs in bold text were added
to provide overlap between the gRNA and destination vector.

| | gRNA Reverse | Gene |
|---|---|---|
| gRNA Sequence (5′→3′) | Complement Sequence (5′→3′) | target |
| CACCGCTACACACGATCGTTAGAG | AAACCTCTAACGATCGTGTGTAGC | Gne |
| CACCGTTCGAGTGATGCGGAAGAA | AAACTTCTTCCGCATCACTCGAAC | Gne |
| CACCCGGCATCTAGAAGCTCAGCG | AAACCGCTGAGCTTCTAGATGCCG | Renbp |
| CACCTGTGCTAGAGAATGTATCAG | AAACCTGATACATTCTCTAGCACA | Renbp |

TABLE 5
Primers used to amplify a region of genomic DNA near the Cas9 cut site of
each gene target.

| | Amplification | Length | Tₘ | GC content |
|---|---|---|---|---|
| Primer sequence (5′→3′) | site | (base pairs) | (° C.) | (%) |
| GTAACAGTGGGAATCAGGTAGAA | Gne Exon 2 | 23 | 54.0 | 43.5 |
| AGCAAGAGCCGTGAAGTAAA | Gne Exon 2 | 20 | 54.3 | 45.0 |
| ACTGTGGTATCCTACCCTATCC | Gne Exon 4 | 22 | 55.0 | 50.0 |
| CGAACTCCACAGCTGCTATT | Gne Exon 4 | 20 | 55.0 | 50.0 |
| CACTCCCATGACCAGGAATATG | Renbp Exon 3 | 22 | 55.1 | 50.0 |
| GGTGAGGGCTTGGAATATACTG | Renbp Exon 3 | 22 | 55.1 | 50.0 |

### Results

### Gne and Renbp Deletion

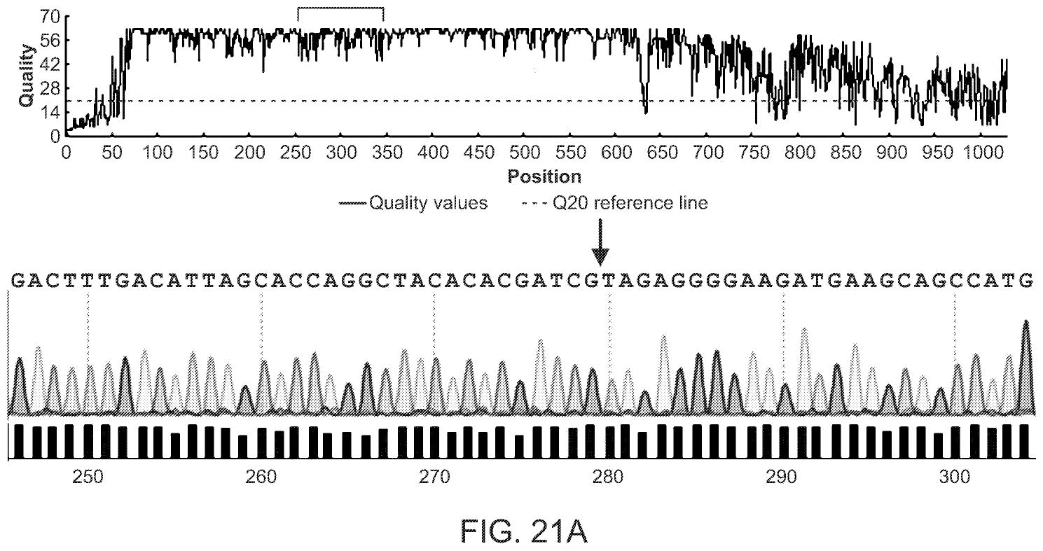

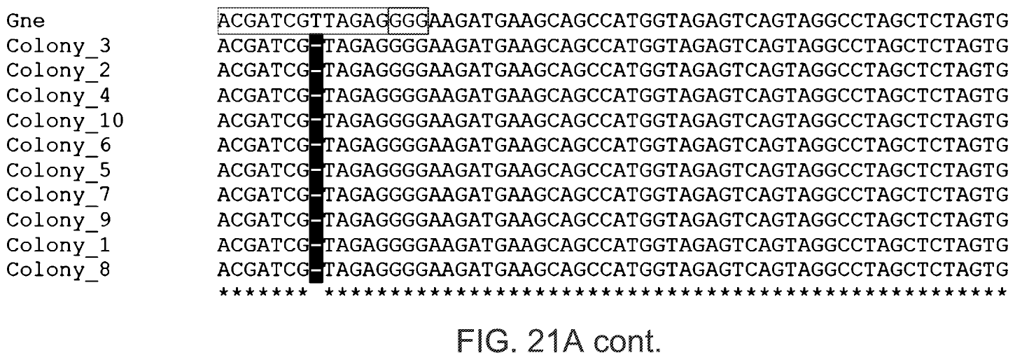

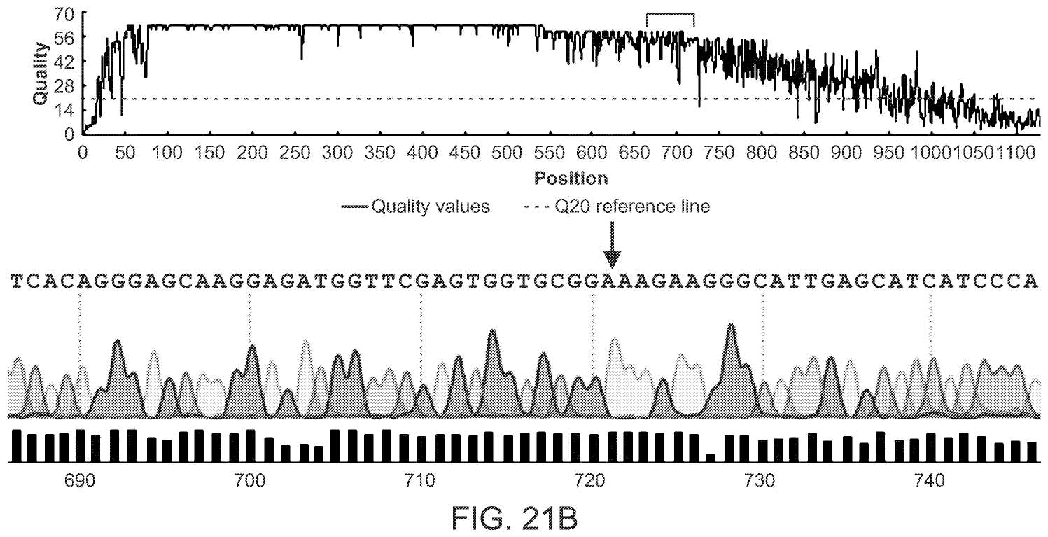

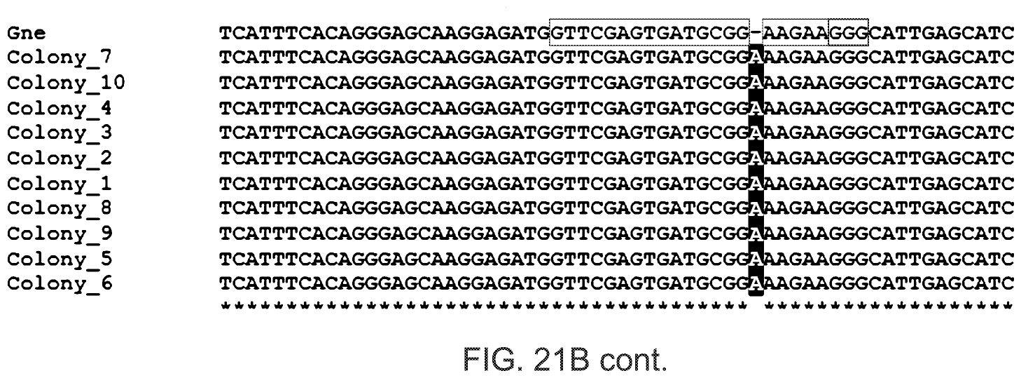

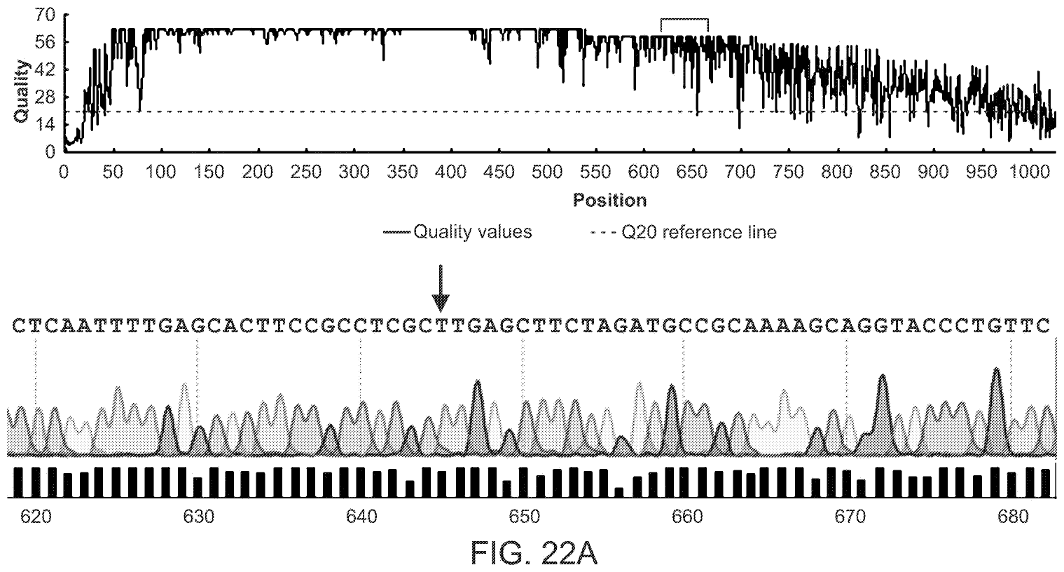

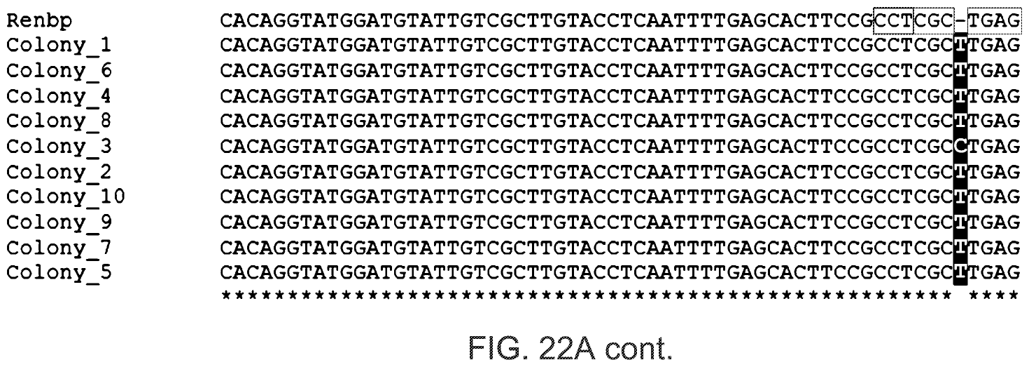

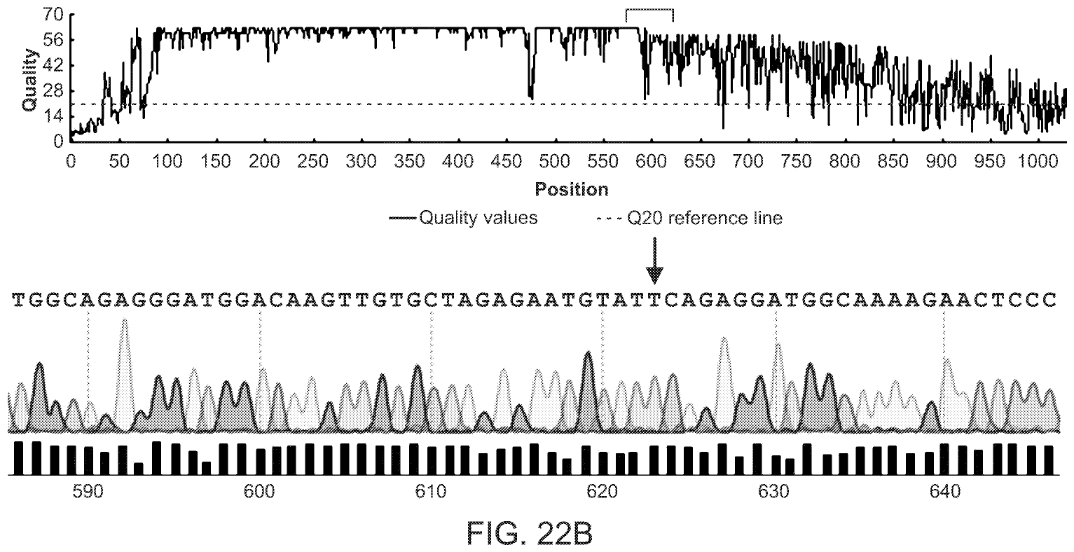

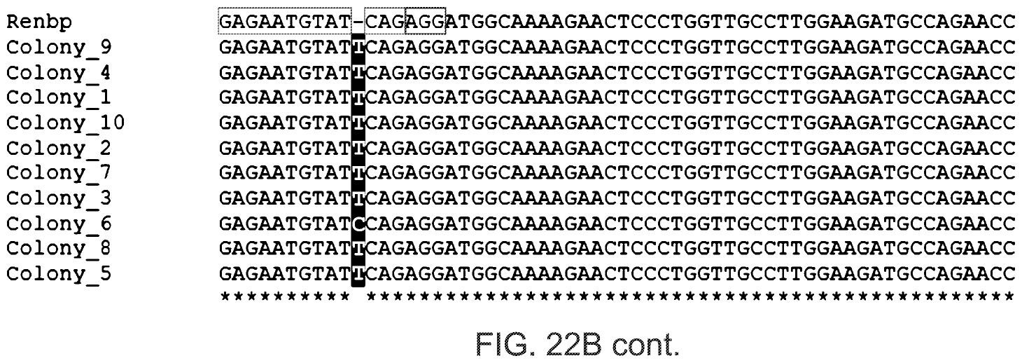

Verification of successful disruption of the targeted exons in CHO cells is provided in FIG. 21 for Gne and FIG. 22 for Renbp. The resulting cell lines are referred to as CHO^{Gne-} and CHO^{Renbp-}, respectively.

### Functional Consequences of GNE Knock Out

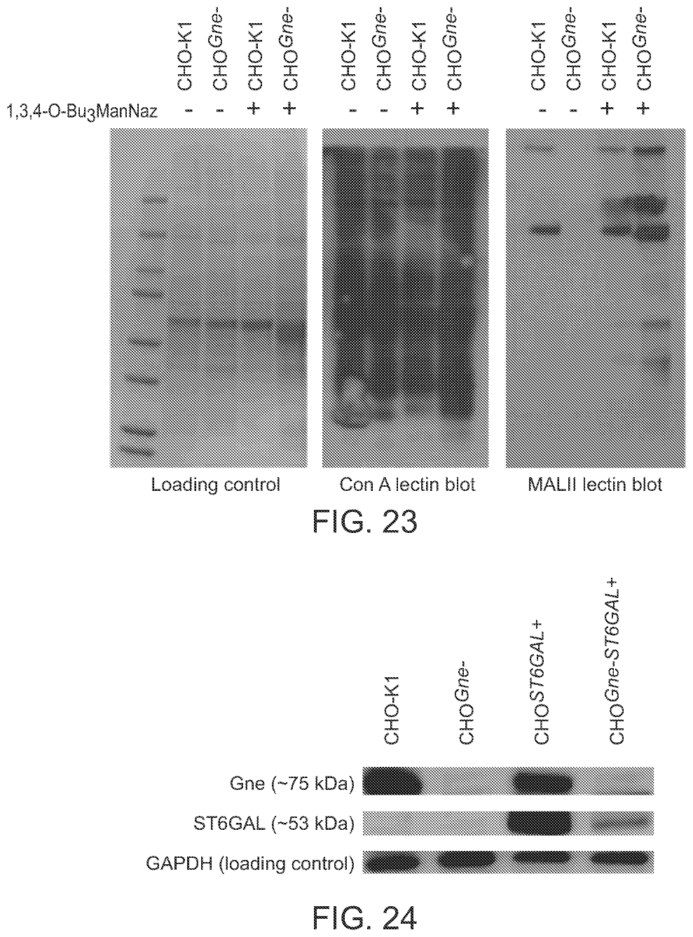

The functional consequences of the knockout of these sialic acid pathway-supplying enzymes was first evaluated in the CHO^{Gne-} cells (Gne was selected for initial characterization because it plays a dominant role in supplying flux into sialic acid biosynthesis compared to Renbp). Secreted proteins were used to demonstrate lack of sialylation in the CHO^{Gne-} cells, as shown in FIG. 23. In this experiment the cells were transferred to serum free media and the secretome was collected after 24 hours and analyzed by gel electrophoresis. Commassie staining (left panel) showed the secretion of a wide range of proteins; Con A lectin staining (middle panel) showed that these proteins were ubiquitously glycosylated, while MAA II staining (right panel) showed that only a small subset of the proteins were α2,3-sialylated. The MAA II blot, however, showed important feature of the CHO^{Gne-} cells including the absence of sialylation in these cells (second lane). Upon treatment with 1,3,4-O-Bu₃ManNAz, a metabolic precursor for non-natural azido-modified sialic acids, only a small gain in signal was observed for the parent cells (c.f., lanes 1 and 3). This result is consistent with competition of flux of endogenously-produced ManNAc into the sialic acid biosynthetic pathway that suppresses the incorporation of the non-natural analog. Finally, a key result shown in FIG. 23 is shown in the fourth lane, where signal from the 1,3,4-O-Bu₃ManNAz-treated CHO^{Gne-} cells is noticeably enhanced consistent with the improved incorporation predicted in the absence of competing flux of endogenously produced ManNAc.

(c) “Knock in” of ST6GAL1 into CHO^{Gne-} Cells

### Rationale

The production of genetically modified CHO cells that over-express the human α2,6-sialyltransferase ST6GAL1 has already been reported but not in the context of cells such as the CHO^{Gne-} line that have been complementarily engineered for control of metabolic flux into the sialic acid biosynthetic pathway. To demonstrate the feasibility of this approach, we have created ST6GAL1 expressing CHO^{Gne-} cells.

### Methods and Results

CHO^{Gne-} cells were seeded in a 6-well plate at 150,000 cells per well in 1 mL of DMEM F-12 media supplemented with 10% FBS and allowed to incubate over night. Approximately 24 hours after plating, the cells were washed, fed with fresh serum-free Opti-MEM media and transduced with ST6GAL1 lentiviral particles. For lentiviral transduction, different multiplicities of infections (MOI; the number of transducing lentiviral particles per cell) were used. An appropriate volume of CD75(ST6GAL1) (NM_173216) Human Tagged ORF Clone Lentiviral Particle (Origene, RC203776L3V) corresponding to an MOI, polybrene (final concentration 8 μg mL-1), and culture media (to a final volume of 500 μL) were added to appropriate wells. The transduced cells were incubated for 18-20 hours with 5% CO₂ in a humidified environment at 37° C. The ST6GAL1 lentiviral particles contain a Myc-DDK tag and the puromycin selection gene. Twenty-four hours after transducing the CHO cells with the ST6GAL1 lentiviral particles, the media was replaced with fresh media supplemented with 10% (v/v) FBS and penicillin-streptomycin. Forty-eight hours post-transduction the CHO cells were treated with 8 μg mL-1 of puromycin to select successfully transduced cells. The puromycin was replaced every third day for a total of 15 days at which point the cells were harvested for western blot analysis to confirm expression of human ST6GAL1 protein (FIG. 24).

### Example 4: Non-Cellular Therapy Designed to Selectively Potentiate Antigen-Specific T_{Reg} Cells Directly in Patients

Regulatory T (T_{Reg}) cells comprise a heterogeneous subset of lymphocytes that inhibit effector T cell activity to orchestrate immune tolerance (1). Recent work has shown that adoptive transfer of T_{Reg} cells can suppress effector T cells to ameliorate autoimmune diseases (1-3). Unfortunately, logistical and manufacturing issues, coupled with concerns about the safety and stability of ex vivo-expanded T_{Reg} cells, impede widespread adoption of this approach (1, 2). Moreover, expansion of antigen-specific T_{Reg} cells, which are significantly more effective in suppressing effector T cell activity than polyclonal T_{Reg} cells, is challenging (4, 5). Thus, there is an unmet clinical need for an off-the-shelf, non-cellular therapy that selectively potentiates antigen-specific T_{Reg} cells directly in patients. Such a therapy would be transformative for treatment of autoimmune diseases such as type 1 diabetes, multiple sclerosis, Crohn's disease, and ulcerative colitis, as well as for transplantation medicine.

To capitalize on the immunosuppressive potential of T_{Reg} cells, low-dose Interleukin-2 (IL-2) therapy has been implemented as a non-cellular alternative to adoptive T_{Reg} therapy (10, 20, 26). IL-2 is a multi-functional cytokine that modulates immune cell differentiation, proliferation, survival, and activity. IL-2 forms a high-affinity (K_{d}˜10 pM) quaternary complex with the IL-2 receptor-α (IL-2Rα, also CD25), IL-2Rβ, and common γ (γ_{c}) chains or an intermediate-affinity (K_{d}˜1 nM) ternary complex with only the IL-2Rβ and γ_{c} chains (7,9). Thus, expression of the non-signaling IL-2Rα subunit modulates cytokine sensitivity whereas IL-2Rβ and γ_{c} mediate signaling (7,10). Since IL-2Rα is abundantly expressed on T_{Reg} cells, but virtually absent from naïve immune effector cells (i.e. CD4⁺ T, CD8⁺ T, and natural killer [NK] cells), low-dose IL-2 treatment preferentially stimulates polyclonal expansion of T_{Reg} over effector cells (6,7). Extensive preclinical and clinical work demonstrates that low-dose IL-2 effectively promotes T_{Reg} expansion; however, IL-2 also expands effector cells, which leads to undesirable off-target effects and toxicities (6,8). Development of a ‘biased’ version of IL-2 that potentiates activity of T_{Reg} but not effector cells would represent a monumental advance for autoimmune disease therapy.

### Materials and Methods

Identification of solvent-exposed loops for introducing minimally disruptive N-glycans. To preserve antibody function, solvent-exposed loops distal to the antigen-binding region of an anti-hIL2 antibody, termed F5111 (Trotta E. et al., Nat Med. 2018 July; 24(7):1005-1014. PMID: 29942088), were identified as potential sites for introducing glycosylation using the protein visualization software Pymol. These loops were predicted to have high solvent exposure to increase the likelihood of glycosylation and the size of the potential glycan modification.

Selection of N-linked glycosylation consensus sequence sites within loops of interest. Using a Python script, a sliding window method was employed to identify all possible sites for insertion of an N-linked glycosylation sequence. N-linked glycosylation sites can be engineered into a protein by modifying existing sequences to code for the Asn-X-Ser/Thr consensus sequence, where X is any amino acid except proline (Pro). For each loop of interest, every amino acid triplet was enumerated to produce a set of possible N-linked glycosylation positions. Amongst these triplets, all single and double amino acid substitutions were considered as potential sites for N-linked glycosylation sequence insertion. Triple amino acid substitutions were not considered to avoid potential disruption to the F5111 antibody structure. The output of this process was a series of modified loops containing the N-X-S and N-X-T motifs.

Computational screening of potential sites for engineered N-linked glycosylation sequence insertion. The NetNGlyc 1.0 Server, an online N-linked glycosylation prediction server, was used to estimate the likelihood that each of the possible engineered glycosylation sites within the F5111 antibody would be successfully glycosylated. Sites with low likelihood of glycosylation (glycosylation likelihood of <0.55) were discarded.

Computational modeling of the engineered glycosylation site. Using either the Pymol mutagenesis wizard or the Rosetta software package, the structures of each engineered glycosylation site within the F5111 antibody were modeled to: (i) avoid steric interference with the antibody itself; (ii) avoid steric interference with IL-2 antigen recognition; and (iii) avoid steric interference with other proteins that form complexes with the antibody.

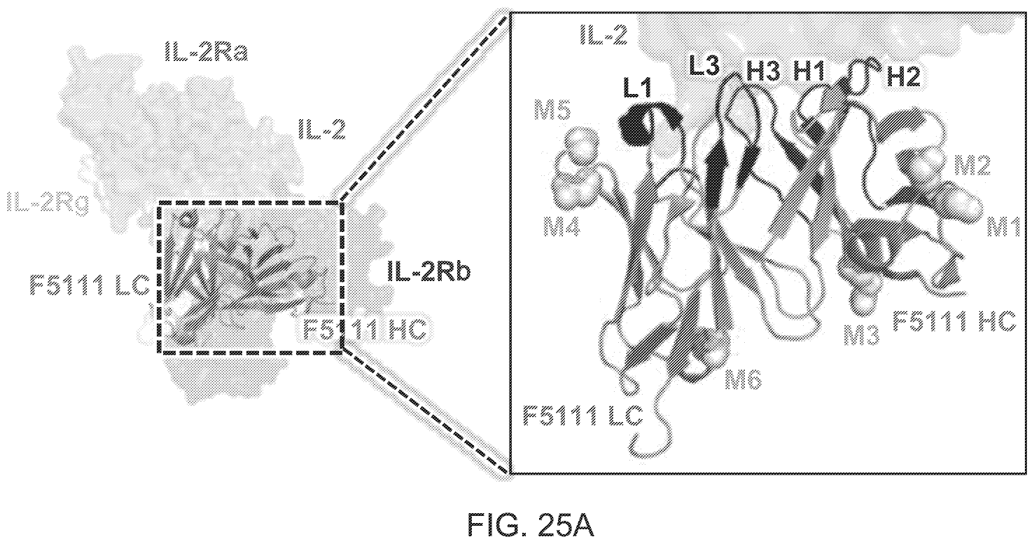

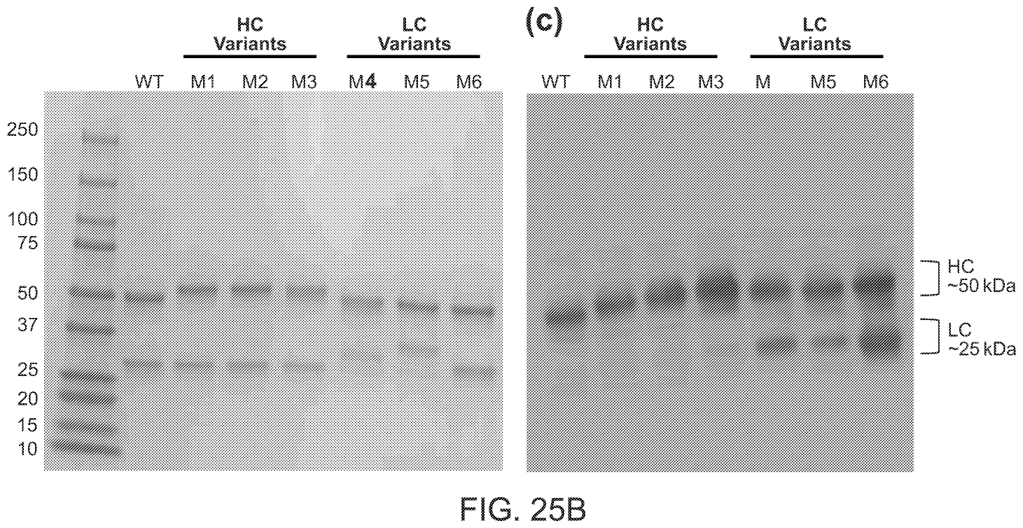

Selection of potential sites for introduction of glycosylation sequences. For the F5111 antibody, eleven solvent-exposed loops containing 62 possible N-linked glycosylation sequences were identified using a sliding window method. This initial set of sequences was reduced down to 36 by considering only those sequences with a likelihood of glycosylation greater than 55% (predicted by NetNGlyc 1.0 server). From this set of 36, we selected 6 mutants (3 heavy chain mutations and 3 light chain mutations) based on structural modeling and steric considerations (FIG. 25A).

Design of F5111 antibody variants with engineered N-linked glycosylation sites. Commercial QUIKCHANGE® mutagenesis kits (Agilent) were used to create plasmid vectors containing mutated DNA sequences encoding the F5111 glycovariant heavy chain (HC) and light chain (LC) sequences. The HC sequences for the F5111 human immunoglobulin G1 (IgG1) and the LC sequences for the human lambda light chain were separately cloned into the gWiz vector (Genlantis). Likewise, the 6 glycoengineered mutants of F5111 (denoted M1-M6) were also separately cloned into the gWiz vector. The resulting 7 antibodies were expressed recombinantly in Chinese hamster ovary (CHO) cells (Thermo Fisher Scientific) via transient co-transfection of plasmids encoding the HC and LC (at a 2:1 ratio of HC:LC DNA), following manufacturer protocols.

Expression and purification of F5111 antibody variants with engineered N-linked glycosylation sites. The M1-M6 glycovariants of the F5111 antibody were expressed via transient transfection of CHO cells, purified via protein G affinity chromatography, and subjected to SDS-PAGE analysis (FIG. 25B). Based on molecular weight shifts apparent under reducing conditions, all 3 glycovariants with engineered N-linked glycosylation sites in the LC (M4, M5, and M6) exhibited an increase in the size of the LC, indicative of successful glycosylation of these mutants. Molecular weight shifts of the 3 glycovariants with engineered N-linked glycosylation sites in the HC (M1, M2, and M3) were more subtle due to the larger size of the HC, as well as the native N-linked glycosylation site found in the constant region of all antibody HCs. Nonetheless, there appear to be increases in the HC sizes for M1, M2, and M3 relative to the wild type F5111 antibody, providing evidence of successful glycosylation of these mutants. Con-A lectin blots verified glycosylation of these engineered F5111 glycovariants (FIG. 25C).

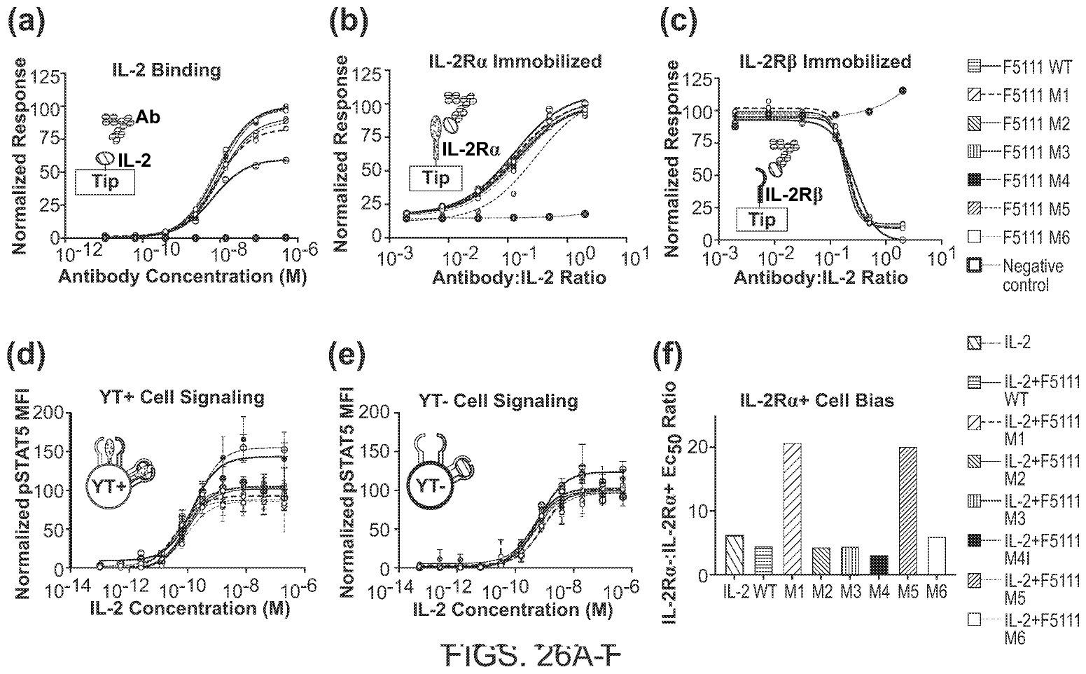

F5111 glycovariants show similar binding and functional properties to parent mAb. Biolayer-interferometry (BLI) studies using immobilized IL-2 demonstrated that glycovariants bound the target cytokine with similar affinity and kinetic properties compared to the wild type antibody (FIG. 26A, Table 6). F5111 biases the IL-2 cytokine by sterically blocking interaction with the IL-2Rb subunit and imposing mild allosteric disruption on the IL-2:IL-2Ra interaction (Trotta E. et al., Nat Med. 2018 July; 24(7):1005-1014. PMID: 29942088). To demonstrate that the competitive binding behaviors of F5111 were still retained by the glycovariants, BLI studies were conducted in which binding of saturating concentrations of soluble IL-2 to immobilized IL-2 receptor subunits was measured in the presence of F5111 antibody. Like the parent F5111 mAb, all six F5111 glycovariants did not affect the IL-2: IL-2Ra interaction (FIG. 26B, Table 6), but fully blocked the IL-2: IL-2Rb interaction (FIG. 26C, Table 6). Interestingly, the glycovariant M5 showed was slightly more competitive with the IL-2:IL-2Ra interaction compared to the parent F5111 mAb and the other glycovariants (FIG. 26B).

To determine whether the functional activity of F5111 was retained by our engineered glycovariants, we made use of the IL-2 responsive human NK cell line denoted YT-1. In this system, IL-2Ra− YT-1 cells (YT−) and IL-2Ra+ YT-1 cells (YT+) serve as surrogates for IL-2Ra_{Low} effector immune cells and IL-2Ra^{High} T_{Reg} cells, respectively. Phosphorylation of signal transducer and activator of transcription (STAT5) in YT− and YT+ cells was quantified as a readout for IL-2 signaling. The parent F5111 biases signaling effects toward cells that express the IL-2Ra subunit, which results in preferential expansion of T_{Reg} cells (Trotta E. et al., Nat Med. 2018 July; 24(7):1005-1014. PMID: 29942088), and consistent with this report, we observed more potent activation of YT+ cells compared to YT− cells by complexes of IL-2 and the wild type F5111 antibody (FIGS. 26D-F, Table 6). Encouragingly, all F5111 glycovariants reproduced the parent antibody's bias toward activation of YT+versus YT− cells. Using the EC₅₀ ratio of IL-2Ra−:IL-2Ra+ as a metric for bias towards IL-2Ra+ cells, we observed that whereas most glycovariants showed similar bias relative to wild type F5111, two glycovariants (M1 and M5) elicited improved bias toward IL-2Ra+ cells (FIG. 26F). This important finding suggests that glycovariants can potentially augment the functional activities of therapeutic antibodies. Overall, these binding and activation studies demonstrate that our pipeline can be used to design atypical N-linked glycans that do not disrupt, and could possibly enhance, the bioactivity an example mAb.

TABLE 6
Fit parameters from binding and signaling pathway
studies with glycoengineered F5111 antibodies.

| | kₒₙ | k_{off} | Kinetic K_{D} | Equilibrium K_{D} | YT+ EC₅₀ | YT− EC₅₀ |
|---|---|---|---|---|---|---|
| | (×10⁻⁶ M⁻¹s⁻¹) | (×10³ s⁻¹) | (nM) | (nM) | (pM) | (pM) |
| IL-2 | N.A. | N.A. | N.A. | N.A. | 60 ± 28 | 370 ± 190 |
| F5111 WT | 0.36 ± 0.00 | 0.22 ± 0.06 | 0.63 ± 0.16 | 6.5 ± 3 | 150 ± 150 | 660 ± 320 |
| F5111 M1 | 0.33 ± 0.00 | 0.38 ± 0.09 | 1.1 ± 0.3 | 7.8 ± 2.7 | 43 ± 27 | 890 ± 690 |
| F5111 M2 | 0.27 ± 0.00 | 0.21 ± 0.08 | 0.76 ± 0.3 | 9.6 ± 3.2 | 98 ± 43 | 440 ± 240 |
| F5111 M3 | 0.31 ± 0.00 | 0.17 ± 0.09 | 0.55 ± 0.28 | 8.3 ± 2.4 | 89 ± 56 | 390 ± 140 |
| F5111 M4 | 0.27 ± 0.00 | 0.14 ± 0.08 | 0.50 ± 0.30 | 9.7 ± 3.0 | 170 ± 370 | 520 ± 260 |
| F5111 M5 | 0.28 ± 0.00 | 0.23 ± 0.10 | 0.82 ± 0.36 | 9.8 ± 2.3 | 39 ± 20 | 790 ± 310 |
| F5111 M6 | 0.37 ± 0.00 | 1.8 ± 0.0 | 5.0 ± 0.1 | 7.0 ± 1.6 | 86 ± 76 | 500 ± 340 |

### Sequences

SEQ ID NO: 5: F5111 and M4-M6 Heavy Chain
Signal sequence - F5111 V_{H} - Human IgG1 C_{H}1, C_{H}2, and C_{H}3
METDTLLLWVLLLWVPGSTGDQLQLQESGPGLVKPSQTLSLTCTVSGGSISSGGYYWS
WIRQHPGKGLEWIGYIYYSGSTYYNPSLKSRVTISVDTSKNQFSLKLSSVTAADTAV
YYCARTPTVTGDWFDPWGRGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIA VEWES
NGQPENNYKTTPPVLDSDGSFFLYSKLTVDKSRWQQGNVFSCSVMHEALHNHYTQKSL
SLSPGK
SEQ ID NO: 6 F5111 and M1-M3 Light Chain
Signal sequence - F5111 V_{L} - Human lambda C_{L}
MRVPAQLLGLLLLWLPGARCGSNFMLTQPHSVSESPGKTVTISCTRSSGSIASNYVQWY
QQRPGSSPTTVIYEDNQRPSGVPDRFSGSIDSSSNSASLTISGLKTEDEADYYCQSYDS
SNVVFGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPT
ECS
SEQ ID NO: 7 M1 Heavy Chain
Signal sequence - F5111 V_{H} - Human IgG1 C_{H}1, C_{H}2, and C_{H}3
Highlighting denotes inserted N-linked glycosylation site

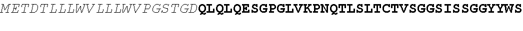

WIRQHPGKGLEWIGYIYYSGSTYYNPSLKSRVTISVDTSKNQFSLKLSSVTAADTAV
YYCARTPTVTGDWFDPWGRGTLVTVSSASTKGPSVFPLAPSSKSTSGGTAALGCLVK
DYFPEPVTVSWNSGALTSGVHTFPAVLOSSGLYSLSSVVTVPSSSLGTQTYICNVNHKPS
NTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSL
SLSPGK
SEQ ID NO: 8 M2 Heavy Chain
Signal sequence - F5111 V_{H} - Human IgG1 C_{H}1, C_{H}2, and C_{H}3
Highlighting denotes inserted N-linked glycosylation site
METDTLLLWVLLLWVPGSTGDQLQLQESGPGLVKPSQTLSLTCTVSGGSISSGGYYWS

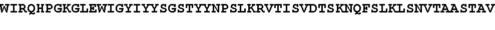

YYCARTPTVTGDWFDPWGRGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSL
SLSPGK
SEQ ID NO: 9 M3 Heavy Chain
Signal sequence - F5111 V_{H} - Human IgG1 C_{H}1, C_{H}2, and C_{H}3
Highlighting denotes inserted N-linked glycosylation site
METDTLLLWVLLLWVPGSTGDQLQLQESGPGLVKPSQTLSLTCTVSGGSISSGGYYWS
WIRQHPGKGLEWIGYIYYSGSTYYNPSLKSRVTISVDTSKNQFSLKLSSVTAADTAV

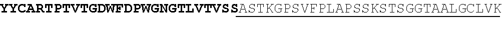

DYFPEPVTVSWNSGALTSGVHTFPAVLOSSGLYSLSSVVTVPSSSLGTQTYICNVNHKPS
NTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSL
SLSPGK
SEQ ID NO: 10 M4 Light Chain
Signal sequence - F5111 V_{L} - Human lambda C_{L}
Highlighting denotes inserted N-linked glycosylation site
MRVPAQLLGLLLLWLPGARCGSNFMLTQPHSVSESPGKTVTISCTRSSGSIASNYVQWY

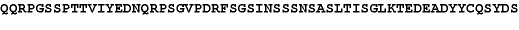

SNVVFGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPT
ECS
SEQ ID NO: 11 M5 Light Chain
Signal sequence - F5111 V_{L} - Human lambda C_{L}
Highlighting denotes inserted N-linked glycosylation site
MRVPAOLLGLLLLWLPGARCGSNFMLTQPHSVSESPGKTVTISCTRSSGSIASNYVQWY

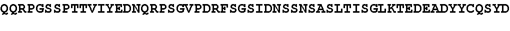

SSNVVFGGGTKLTVLGQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVEKTVAP
TECS
SEQ ID NO: 12 F5111 and M1-M3 Light Chain
Signal sequence - F5111 V_{L} - Human lambda C_{L}
Highlighting denotes inserted N-linked glycosylation site
MRVPAOLLGLLLLWLPGARCGSNFMLTQPHSVSESPGKTVTISCTRSSGSIASNYVQWY

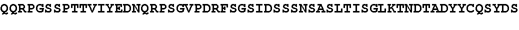

SNVVFGGGTKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGSTVEKTVAPT
ECS

### REFERENCES

1. El Essawy B, Li X C. Type 1 diabetes and T regulatory cells. Pharmacological Research. 2015 Aug. 1; 98 (Supplement C):22-30.
2. Battaglia M, Roncarolo M-G. Immune intervention with T regulatory cells: Past lessons and future perspectives for type 1 diabetes. Seminars in Immunology. 2011 Jun. 1; 23(3): 182-194.
3. Marek-Trzonkowska N, Mysliwiec M, Dobyszuk A, Grabowska M, Techmanska I, Juscinska J, Wujtewicz M A, Witkowski P, Mlynarski W, Balcerska A, Mysliwska J, Trzonkowski P. Administration of CD4+CD25highCD127− regulatory T cells preserves β-cell function in type 1 diabetes in children. Diabetes Care. 2012 September; 35(9):1817-1820. PMCID: PMC3425004.
4. Lee K, Nguyen V, Lee K-M, Kang S-M, Tang Q. Attenuation of donor-reactive T cells allows effective control of allograft rejection using regulatory T cell therapy. Am J Transplant. 2014 January; 14(1):27-38. PMCID: PMC5262439.
5. Tang Q, Vincenti F. Transplant trials with Tregs: perils and promises. J Clin Invest. 2017 Jun. 30; 127(7):2505-2512.
6. Boyman O, Sprent J. The role of interleukin-2 during homeostasis and activation of the immune system. Nat Rev Immunol. 2012 Feb. 17; 12(3):180-190. PMID: 22343569.
7. Liao W, Lin J-X, Leonard W J. Interleukin-2 at the crossroads of effector responses, tolerance, and immunotherapy. Immunity. 2013 Jan. 24; 38(1):13-25. PMCID: PMC3610532.
8. Klatzmann D, Abbas A K. The promise of low-dose interleukin-2 therapy for autoimmune and inflammatory diseases. Nat Rev Immunol. 2015 May; 15(5):283-294. PMID: 25882245.
9. Wang X, Rickert M, Garcia K C. Structure of the quaternary complex of interleukin-2 with its alpha, beta, and gammac receptors. Science. 2005 Nov. 18; 310(5751): 1159-1163. PMID: 16293754.
10. Malek T R. The Biology of Interleukin-2. Annual Review of Immunology. 2008; 26(1):453-479. PMID: 18062768.
11. Boyman O, Kovar M, Rubinstein M P, Surh C D, Sprent J. Selective stimulation of T cell subsets with antibody-cytokine immune complexes. Science. 2006 Mar. 31; 311(5769): 1924-1927. PMID: 16484453.
12. Tang Q, Adams J Y, Penaranda C, Melli K, Piaggio E, Sgouroudis E, Piccirillo C A, Salomon B L, Bluestone J A. Central role of defective interleukin-2 production in the triggering of islet autoimmune destruction. Immunity. 2008 May; 28(5):687-697. PMCID: PMC2394854.
13. Grinberg-Bleyer Y, Baeyens A, You S, Elhage R, Fourcade G, Gregoire S, Cagnard N, Carpentier W, Tang Q, Bluestone J, Chatenoud L, Klatzmann D, Salomon B L, Piaggio E. IL-2 reverses established type 1 diabetes in NOD mice by a local effect on pancreatic regulatory T cells. Journal of Experimental Medicine. 2010 Aug. 30; 207(9):1871-1878. PMID: 20679400.
14. Webster K E, Walters S, Kohler R E, Mrkvan T, Boyman O, Surh C D, Grey S T, Sprent J. In vivo expansion of T reg cells with IL-2-mAb complexes: induction of resistance to EAE and long-term acceptance of islet allografts without immunosuppression. Journal of Experimental Medicine. 2009 Apr. 13; 206(4):751-760. PMID: 19332874.
15. Liu R, Zhou Q, La Cava A, Campagnolo D I, Van Kaer L, Shi F-D. Expansion of regulatory T cells via IL-2/anti-IL-2 mAb complexes suppresses experimental myasthenia. Eur J Immunol. 2010 Jun. 1; 40(6): 1577-1589.
16. Spangler J B, Tomala J, Luca V C, Jude K M, Dong S, Ring A M, Votavova P, Pepper M, Kovar M, Garcia K C. Antibodies to Interleukin-2 Elicit Selective T Cell Subset Potentiation through Distinct Conformational Mechanisms. Immunity. 2015 May 19; 42(5):815-825.
17. Trotta E, Bessette P H, Silveria S L, Ely L K, Jude K M, Le D T, Holst C R, Coyle A, Potempa M, Lanier L L, Garcia K C, Crellin N K, Rondon I J, Bluestone J A. A human anti-IL-2 antibody that potentiates regulatory T cells by a structure-based mechanism. Nat Med. 2018 July; 24(7): 1005-1014. PMID: 29942088.

### Example 5: Improving the Pharmacodynamics and In Vivo Activity of ENPP1, a Therapeutic Enzyme, Through Protein and Glycosylation Engineering

The ectonucleotide pyrophosphatase/phosphodiesterase (ENPP) enzyme family catalyzes phosphoryl-transfer reactions on extracellular phosphoanhydrides. The products of these reactions, such as ATP, lysophosphatidic acid, and pyrophosphate, are essential extracellular signaling molecules that govern whole organismal fate through the regulation of essential biological functions such as angiogenesis (1-3), cell motility (4, 5), tumor metastasis (6-10), bone mineralization (11-14), vascular calcification (15-17), and hemostasis (18). Non-enzymatic phosphoryl-transfer reactions that produce these critical signaling molecules have some of the slowest reaction rates known; as a result, living systems depend on tremendous acceleration from ENPPs that serves as a critical biologic catalyst. Quantitatively, ENPPs accelerate reaction rates by as much as 1027-fold (19) and, as outlined below, a devastating impact on normal biological function occurs with ENPP deficits.

ENPP1 generates nucleoside 5′ monophosphates and phosphoanhydride pyrophosphate (PPi) from extracellular nucleotide triphosphates. ENPP1 is the only human enzyme which synthesizes extracellular PPi, and deficiencies of ENPP1 result in a range of human disorders. Levels of PPi in healthy individuals have been measured at 2.0-2.2 μM in patients (20), compared to patients with heterozygous ENPP1 deficiency who exhibit half-normal plasma PPi (21), while homozygous ENPP1 deficient patients exhibit nearly absent levels of plasma PPi-typically below 250 nM (15, 16).

The clinical consequences of ENPP deficiency are severe and therapeutic options are limited because the bioactive metabolic products of this enzyme (PPi and nucleoside 5′ monophosphates) are not bioavailable. To improve ENPP1 to meet or exceed a once a month or better benchmark for dosing frequency provided impetus to implement a series of glycosylation and protein engineering strategies that were further combined with production in a novel biomanufacturing platform. This resulted in an increase in serum half-life by ˜5.5-fold for ENPP1 (204 vs. 37 h) and 13-fold for AUC (45,000 vs 3,400), which is the critical metric for in vivo bioavailability. Importantly, these improvements were achieved without compromising the disease-modifying enzyme activity of ENPP1.

### Methods

### Mice

All animal procedures complied with the US National Institutes of Health guide for the care and use of laboratory animals and were approved by the Animal Care and Use Committee of Yale University. Animal care and maintenance were provided through Yale University Animal Resource Center. Pharmacokinetic analysis was performed on 6-week-old C57BL/6J Male mice purchased from The Jackson Laboratory. Pharmacodynamic analysis was performed on Enpp1^{asj/asj} mice (Jackson Laboratory stock number 012810)

### Three-Dimensional Structural Modelling

A model of the tertiary structure of ENPP-1 was based on close homologs in the Protein Data Bank PDB derived from the RaptorX protein structure prediction server(43). Protein structure visualization and assessment were carried out using PyMOL3 version 2.0.7.

### Cloning and Protein Expression and Purification

A codon optimized synthetic gene of a secretion optimized extracellular ENPP1 construct previously described (24) was purchased from Invitrogen GeneArt (ThermoFischer Scientific, Waltham, MA) and sub-cloned into pFUSE-h IgG₁-Fc1 (InvivoGen, San Diego, CA) resulting in an “in frame” c-terminal fusion with a human Fc Domain of IgG₁ hereafter referred to as ENPP1-Fc or construct #770. All mutations introduced into construct #770 were engineered using the QuikChange II XL site directed mutagenesis kit from Agilent Technologies following the manufacturer's protocols.

CHO cells were stably selected for either ENPP1-Fc or ENPP1-Fc and α-2,6-sialyltransferase expression and were adapted for suspension growth in PeproGrow serum free media from PeproTech (Rocky Hill, NJ). Cells were expanded with or without 100 μM 1,3,4-O-Bu₃ManNAc supplementation using conditions previously optimized by the Yarema group (44), and secreted protein was purified to homogeneity as previously described (24). Purified proteins were stored as frozen stocks in PBS at −80° C.

### Glycan Analysis

Glycan analysis was performed at the Complex Carbohydrate Research Center (Athens, Ga.). Approximately 400 μg of each sample was dissolved in 50 μL digestion buffer (50 mM aq. NH₄CO₃), subjected to reduction (25 μL, 25 mM DTT, 45 min, 45° C.) followed by alkylation (25 μL, 90 mM Iodoacetamide, 45 min, RT) in the dark. The samples were then dialyzed against nanopure water using 4 kDa tube dialyzer overnight. Then N-glycans were released by enzymatic cleavage with PNGase F at 37° C. and purified of any contaminants with a C18 Sep-Pak cartridge. The carbohydrate fraction was eluted with 5% acetic acid and dried by lyophilization. Released N-linked oligosaccharides were permethylated by using methyl iodide on DMSO/NaOH mixture, dried with nitrogen gas and profiled by MALDI-TOF analysis. A portion of the samples (20 μg) after reduction/alkylation were treated with sequencing grade trypsin and analyzed by LC-MS/MS. The glycopeptides were detected, assigned and quantitated by search using Byonic software and manual method.

For HPAEC-PAD analysis samples were dissolved in water, and an aliquot (˜160 μg) was allocated for neutral and amino sugars and a similar amount of aliquot was taken for sialic acid analysis. The aliquots for neutral and amino sugars were hydrolyzed with 2 M trifluoroacetic acid (4 h at 100° C.), whereas the aliquots for sialic acids were hydrolyzed with 2 M acetic acid (3 h at 80° C.). The monosaccharides were analyzed by HPAEC-PAD using a Dionex ICS3000.

### Enzyme Kinetics

Cell media from transiently transfected CHO cells was analyzed for ENPP1 enzyme activity by diluting 10 μL of conditioned media into 90 μL of buffer containing 1 M Tris pH 8.0, 50 mM NaCl, 20 μM CaCl₂), 20 μM ZnCl₂, and 1 mM thymidine 5′-monophosphate p-nitrophenyl (TMP-pNP) from Sigma-Aldrich (cat #T4510). The mean velocity of the chromogenic product was measured in triplicate in the linear range using a Synergy Mx microplate reader with Gen5 software, and reported as change at absorbance per min (a.u. min⁻¹) at 405 nm light.

The Michaelis-Menten kinetic constants of ENPP1-Fc glycoforms with TMP-pNP as substrate were obtained on a Synergy Mx microplate reader using Gen5 software. Briefly, enzyme kinetics were measured at room temperature at two concentrations (50 ng and 100 ng) in 100 mL of a buffer containing 250 mM Trizma pH 9.4, 500 mM NaCl, 0.05% Triton X-100. The absorbance at 405 nM was recorded every 4 seconds immediately after the addition of 8 concentrations of the TMP-pNP substrate (50, 100, 200, 300, 400, 500, 750, and 1000 μM) and the mean initial velocity of change of absorbance in unit time was derived from the first 2 time points and TMP-pNP concentration dependence of the initial velocity was used to calculate the Vmax and Km using the equation Vinit=Vmax*[TMP−pnP]/(Km+[TMP−pNP]). The data was analyzed with GraphPad Prism 8.

### Pharmacokinetics

Six-week-old C57BL/6J male mice were injected with a single subcutaneous dose of purified enzyme and blood was collected up to 4 times retro-orbitally at the various time points. The blood was centrifuged at 900×g for 10 min from which the plasma was transferred to a new tube and frozen. Plasma ENPP1-Fc activity in plasma was quantified using the enzyme velocity assay on 2 μL of plasma diluted into 98 μL of TMP buffer, and was calculated from velocity values in the linear range as absorbance per min (a.u. min⁻¹) at 405 nm light. For half-life calculations the velocity values were converted to percent activity and plotted in GraphPad Prism 8. Pharmacokinetic parameters were calculated by plotting the fractional enzymatic activity (F) of ENPP1-Fc at each time point to determine the elimination (kₑ) and absorption (kₐ) rate constants by fitting the experimental curves to equation 1.

$$F = \frac{{k}_{a}}{( {k}_{a} - {k}_{e} )} [ {e}^{- {k}_{e} t} - {e}^{- {k}_{a} t} ]$$

### Eq. 1: Relationship Between Systemic Fractional Concentration and Time of a Drug Administered to a Subcutaneous Depot in a Single Injection.

Samples of blood were collected at 4 time points that initially were between 12 and 75 h post injection; later, with PK improvements, time points were taken between 21 and 263 h post injection. The initial fractional activity from the first bleed was assigned a fractional activity of 0.9. Mice were also immunosuppressed with an i.p. injection of Anti-CD4 clone Gk1.5 24 h before s.c. injection of enzyme to mitigate any negative immune responses mounted against the longer lasting therapeutic. The data was analyzed and visualized with GraphPad Prism 8.

### Data Set Availability

The data that support the findings of this study are available from corresponding author upon reasonable request.

### Results

### Design Criteria for Addition of New N-Glycans to ENPP1-Fc

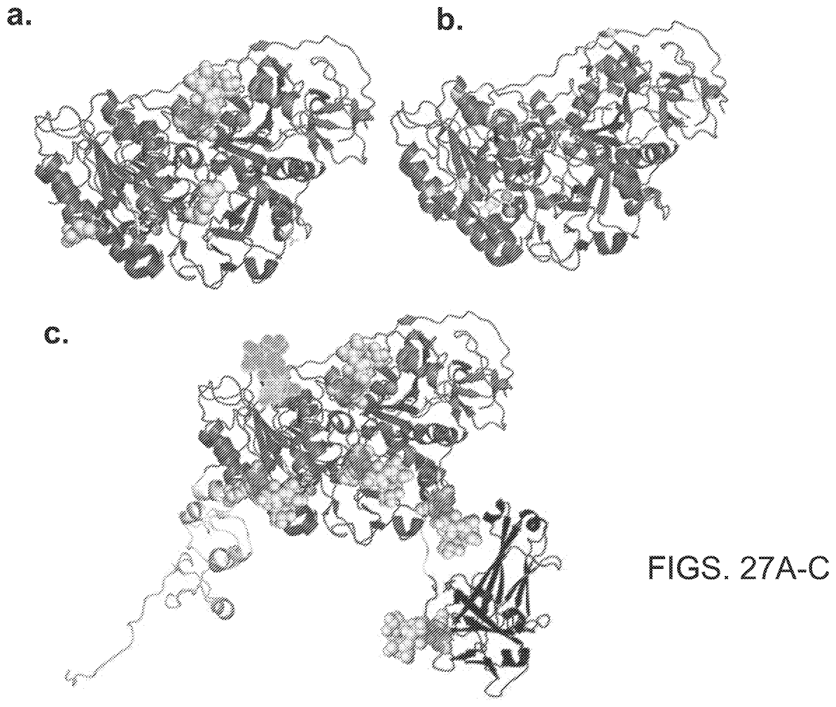

Based on evidence that therapeutic proteins benefit from the addition of N-glycans when none are originally present (insulin (32)) as well as when a protein is already glycosylated (erythropoietin (33)), this strategy was applied to ENPP1. Although the general principle of adding an N-glycan to a therapeutic protein is established, at present the exact site to add the glycan for maximal benefit can only be determined empirically. Nevertheless, a semi-rational approach was used, which first required knowledge of existing glycans. The crystal structure of murine Enpp1 exhibits 4 glycans (34) which was reasoned that these were also present in (highly homologous) human ENPP1. In addition, human ENPP1 has six additional N-glycan consensus sequences (N-GCS) that, (FIG. 27A), although their glycosylation status in unknown, can be incorporated into structural models that integrate clinical and genetic data obtained from GACI patients to help design improved glycoforms/glycovariants. To assist the protein design, structural models were created, integrating the above glycosylation sites with the locations of ENPP1 loss of function mutations obtained from the clinical and genetic data reported in GACI patients (FIG. 27B). The protein sequence database was used to identify N-GCS in ENPP family members (ENPP2-ENPP7) from all mammalian species with available protein sequences, and modeled these sites onto mouse Enpp1 (PDB 4GTW). Using these tools, a list of potential N-GCS sites was established by avoiding sites near inactivating ENPP1 mutations (FIG. 27B) and sites that would disrupt the disulfide bonding pattern.

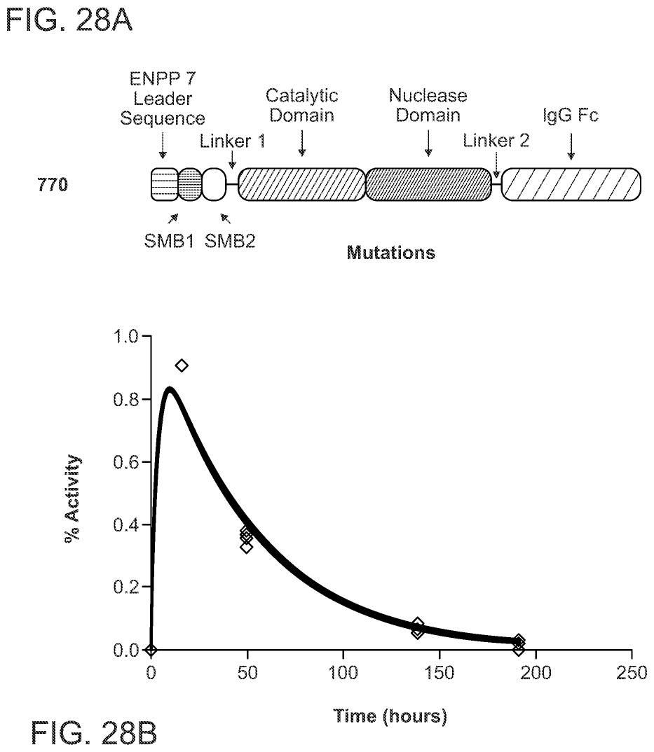

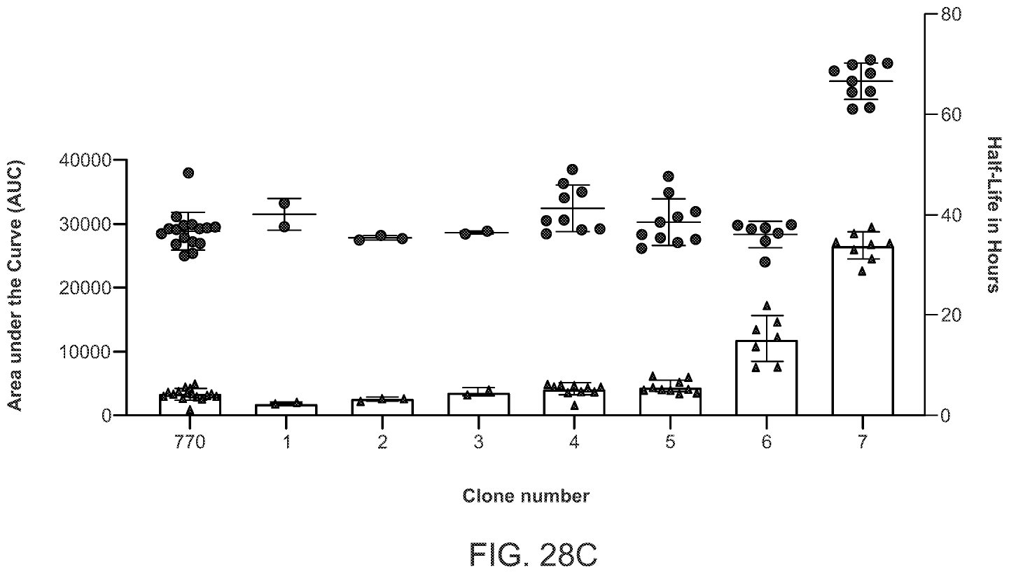

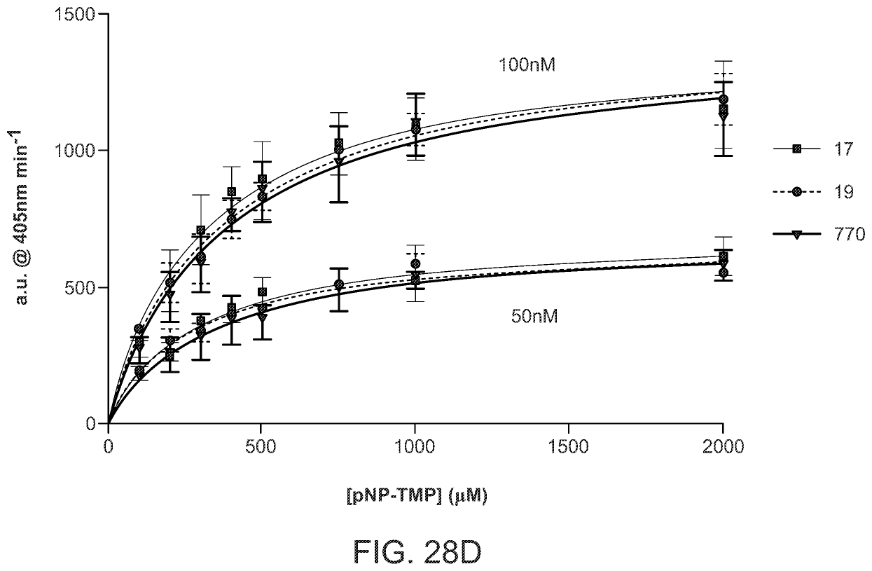

Disordered sequences were targeted on the exterior surface of ENPP1-Fc that permitted the introduction of an N-GCS through a single amino acid substitution as sites to add N-glycans. If the location of a proposed N-GCS were near inactivating mutations in GACI patients or interfered with the ENPP1 disulfide bonds, the modifications were not pursued. By using these criteria, we selected 31 possible sites to add N-glycans, attempting to individually sample the entire surface area of ENPP1. Each of these 31 N-GCS were introduced individually or in some cases in combination the parent ENPP1-Fc (construct #770, FIG. 28A) via site directed mutagenesis resulting in a final pool of 53 ENPP1-Fc glycoforms. Protein production levels from transient expression in Chinese hamster ovary (CHO) cells were measured for each form of ENPP1-Fc and enzymatic activity was quantitated as described in methods. The nine most promising N-GCS isoforms, as determined from their reaction velocities, were stably selected as single alterations or as double mutants by establishing adherent individual CHO K1 cell clones, and the glycoforms were individually expressed and purified to homogeneity.

### Pharmacokinetic Effects of Adding N-Glycans to ENPP1-Fc

Pharmacokinetics of the glycoengineering forms of ENPP1-Fc were tested in C57BL6 mice using a single subcutaneous injection, with doses varying from 5 mg/Kg for the early constructs to 0.3 mg/Kg for the more potent latter constructs. Representative plots for the parent ENPP1-Fc (clone 770) are displayed in FIG. 28B, yielding a half-life of 37 h and an area under the curve (AUC) of 3,382. The effect of adding an additional N-GCS to most of the ENPP1-Fc glycoforms was modest with the notable exception of the I256T mutation, which was designed to add an N-glycan at asparagine 254, which is located in the catalytic domain near the active site. This N-glycan, which is present at the analogous position in human ENPP3, increased the bioavailability of ENPP1-Fc (i.e., AUC) by ˜8 fold and its half-life by a factor of 1.8 (construct 7, Table 7 and FIG. 28C).

TABLE 7
Pharmacokinetic effects of added N-GCS

| | | | | | Half- | |
|---|---|---|---|---|---|---|
| | Signal | | Nuclease | | life | |
| Construct | Sequence | CatalyticDomain | Domain | Linker2 | (Hours) | AUC |
| 770 | | | | | 37 | 3382 |
| 1 | | | E592N | | 40 | 1935 |
| 2 | | K369N/I371T | | | 35 | 2561 |
| 3 | C25N K27T | | | | 36 | 3636 |
| 4 | V29N | | | E864NL866T | 41 | 4134 |
| 5 | C25N K27T | | S766N | | 38 | 4537 |
| 6 | C25N K27T | | | E864N | 36 | 11997 |
| | | | | L866T | | |
| 7 | | I256T | | | 66 | 26596 |

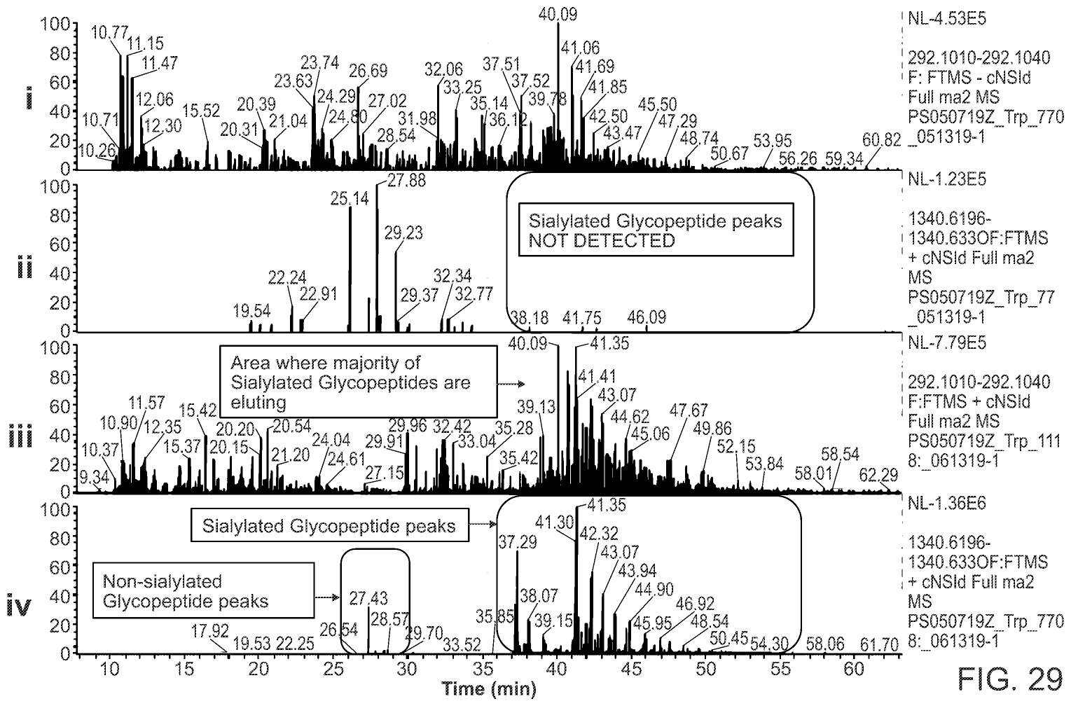

To determine whether the ˜8-fold increase in bio-availability resulted from improved pharmacokinetics or a gain of function in catalytic activity, we compared the Michaelis-Menten kinetic constants of the parent ENPP1-Fc with two I256T containing constructs (clone 17 and clone 19) at two different concentrations, and found no significant differences between the Km or Kcat of either enzyme (FIG. 28D). Next, the presence of a glycan at the Asn254 of ENPP1-Fc clone 19 was confirmed using mass spectrometry. Specifically, the I256T mutation was located in the digested peptide fragment ²⁴¹SGTFFWPGSDVEINGTFPDIYK²⁶². and glycosylation at position Asn254 was indicated by the gain of sialyl glycopeptide peaks (FIG. 29), compared to parent ENPP1-Fc, which lacks the I256T mutation. These findings demonstrate that the increased half-life and AUC induced by the additional N-glycan at residue 256 resulted from enhanced PK properties rather than enhanced catalytic function of the enzyme.

PK Effects of Optimizing pH-Dependent FcRn Recycling

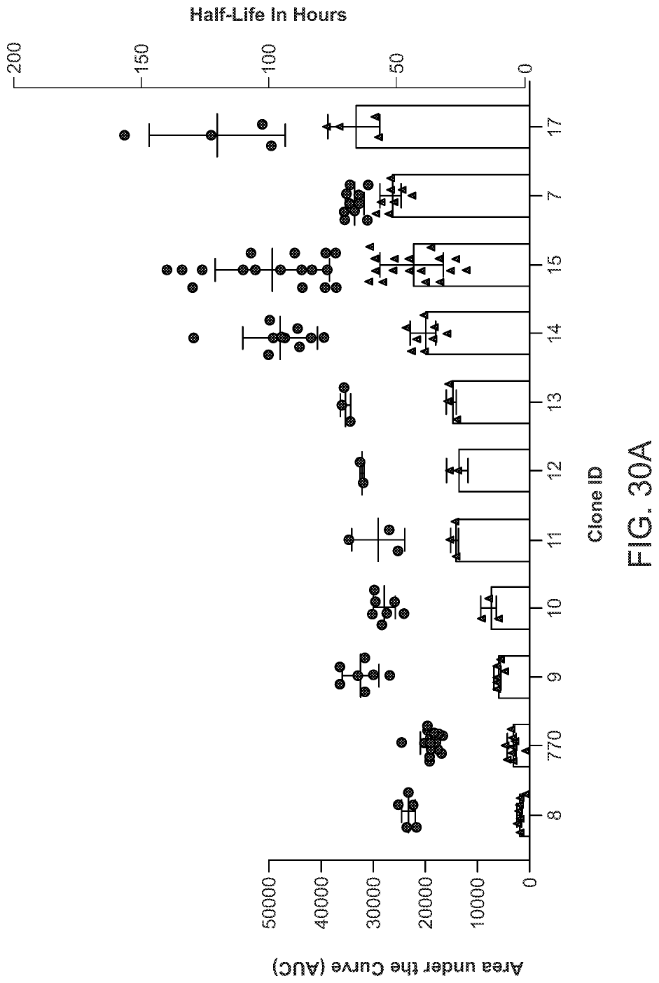

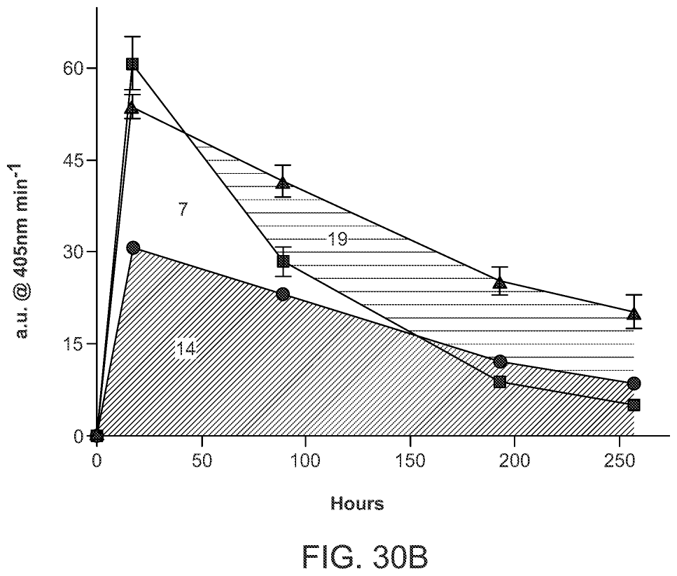

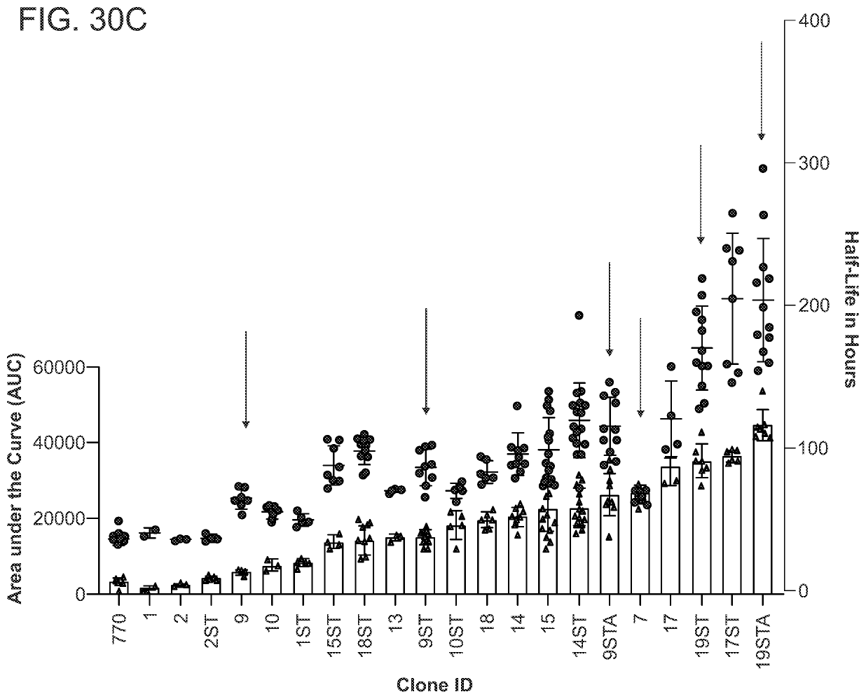

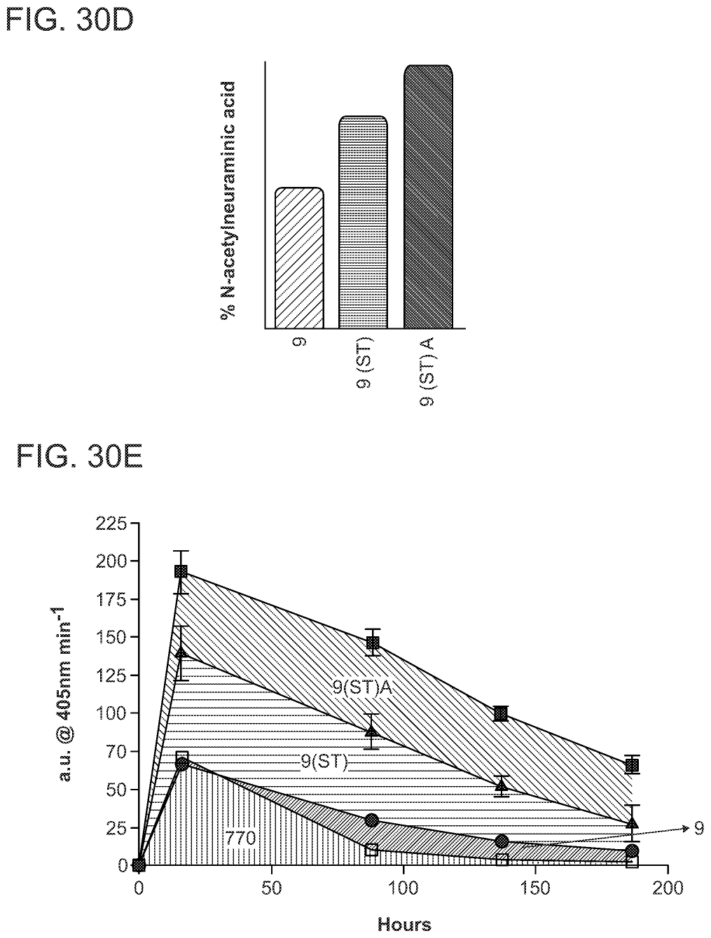

Mutations in the Fc domain of therapeutic antibodies are known that enhance the pH dependent interactions of Fc with FcRn and thereby extend circulatory half-life (35). The effect of two sets of such Fc mutations—H433K/N434F (i.e., “HN” mutations) and M242Y/S254T/T246E (“MST” mutations) were examined. Both Fc variants were combined with N-GCS isoforms to test the combined effect of hyperglycosylation and enhanced FcRn recycling. In general, the Fc MST mutations increased biologic exposure (AUC) to a greater degree than the Fc HN mutations (Table 8, FIG. 30A). For example, adding the MST mutations to parent ENPP1-Fc (construct 14) increased AUC by ˜6-fold and half-life by ˜2.6-fold, in comparison to the HN mutations (constructs 9-12) which increased AUC≤4.1 fold in the presence of additional glycans (Table 8 and FIG. 30A). However, in some cases specific N-GCS mutations decreased bioavailability when combined with the Fc mutations, i.e., the combination of an N-glycan at residue 766 with either Fc mutation was detrimental (MST in construct 8 vs. construct 14 and HN in construct 9 vs. construct 11, Table 8 and FIG. 30A). Reproducibility was assessed by establishing two independent CHO cell clones producing the same ENPP1-Fc glycoform (clones 11 and 12); little variation in the PK of these two clones was observed (Table 8). Finally, the improvement in AUC achieved solely by optimizing FcRn recycling was less than that achieved from adding an N-glycan at residue 254 (clone 7 vs. clone 14, FIG. 30A). PK plots illustrate that Fc optimization enhanced PK by increasing the half-life (decreasing the slope of the velocity vs time curve in FIG. 30B), whereas the N254 N-glycan enhanced PK by increasing drug absorption into plasma i.e., increasing Cmax (greater maximal activity of clone 7 in FIG. 30B). Finally, combining the Fc mutation and the N254 N-glycan increased both the half-life and the Cmax, as observed by the greater maximal activity and reduced slope of the activity vs. time curve in clone 19 (ST).

TABLE 8
Pharmacokinetic effects combining N-GCS and Fc mutations

| | | | | | Half- | |
|---|---|---|---|---|---|---|
| | | | | | Life | |
| Construct | SignalSequence | CatalyticDomain | NucleaseDomain | Fc Domain | (Hours) | AUC |
| 8 | | | S766N | M883YS885T | 45 | 1912 |
| | | | | T887E | | |
| 770 | | | | | 37 | 3382 |
| 9 | V29N | | S766N | H1064K N1065F | 65 | 6047 |
| 10 | C25NK27T | | S766N | H1064K N1065F | 55 | 7735 |
| 11 | V29N | | | H1064KN1065F | 57 | 14506 |
| 12 | V29N | | | H1064K N1065F | 63 | 13812 |
| 13 | V29N | | E592N | M883YS885T | 70 | 14978 |
| | | | | T887E | | |
| 14 | | | | M883YS885T | 95 | 20360 |
| | | | | T887E | | |
| 15 | V29N | | E592N | H1064K N1065F | 99 | 22690 |
| 7 | | I256T | | | 66 | 26596 |
| 17 | V29N | I256T | P534NV536T | M883YS885T | 120 | 33751 |
| | | | | T887E | | |

### PK Effects of “Humanized” α-2,6-Sialyation

One reason that CHO cells are now used for biomanufacturing protein therapeutics is that their glycosylation patterns are sufficiently similar to humans to avoid any overt safety concerns (for example, CHO cells do not produce the immunogenic alpha-Gal epitope (36, 37). Differences between human and hamster cells do exist, however, and these differences can compromise efficacy; for example, standard CHO cells used in biomanufacturing lack α-2,6-linked sialic acids linked to favorable PK properties of therapeutic proteins. To overcome this deficit, a CHO cell line was established, stably expressing human β-galactoside α-2,6-sialytransferase (α-2,6-ST) and evaluated ENPP1-Fc glycoforms produced in these cells. It was found that production of ENPP1-Fc in these cells improved the PK properties as expected; for example, PK increased 12-70% for clones 2 and 14 (Table 9 and FIG. 30C).

PK Effects of Increased Flux-Based Sialylation from 1,3,4-O-Bu₃ManNAc

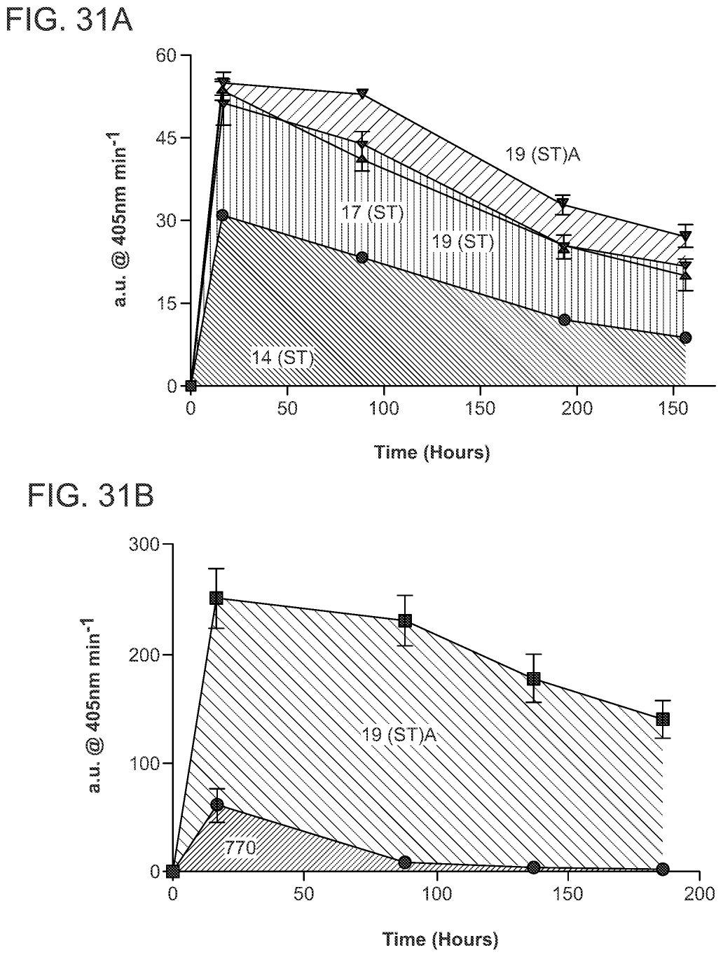

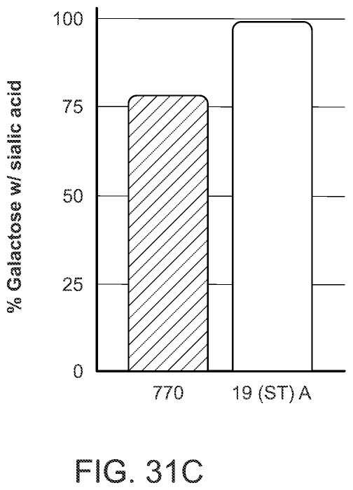

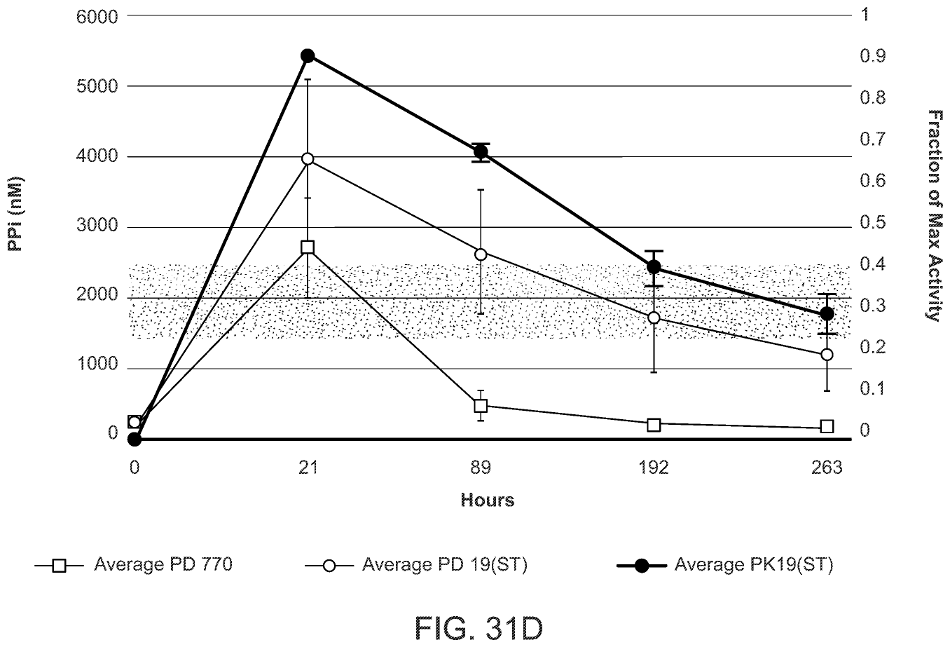

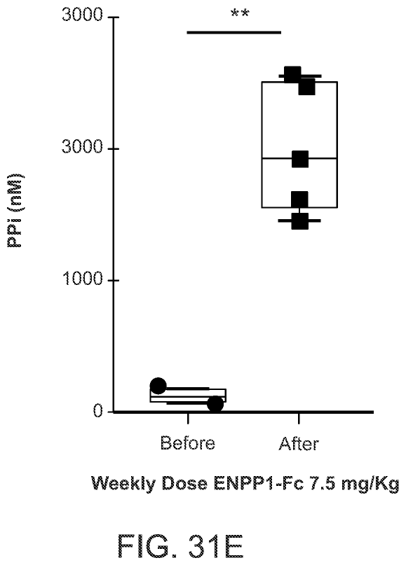

The benefits derived from producing ENPP1 in α-2,6-ST over-expressing CHO cells have two sources. One, as mentioned, is the from the gain of α-2,6-linked sialic acids. A second is that sialyltransferase over-expression increases overall sialylation, which also improves PK properties. To further exploit this putative mechanism, the production cells were supplemented with 1,3,4-O-Bu₃ManNAc, a “high-flux” metabolic precursor that supplies flux into the sialic acid biosynthetic pathway and increases glycoconjugatesialylation (38, 39). This metabolite provided added benefit when combined with the previously described components of the biomanufacturing platform as illustrated by clone 9 (FIG. 30C), which possesses two additional glycan consensus sequons. Specifically, this ENPP1-Fc glycoform exhibited only modestly increased biologic effects when produced in standard CHO cells; its PK increased by 2.6-fold when expressed in CHO cells over-expressing α-2,6-ST; and 1,3,4-O-Bu₃ManNAc supplementation provided an added ˜1.7-fold benefit (clones 9, 9(ST), and 9(ST)A, respectively, Table 9 and FIG. 30E). Overall the combined use of ST6-over-expressing CHO cells and the metabolic precursor for sialylation provided a ˜4.3-fold improvement for clone 9. Finally, expressing clone 19(ST) in media containing the sialic acid precursor 1,3,4-O-Bu₃ManNAc yielded the best performing ENPP1-Fc glycoform, increasing bioavailability by ˜13 fold above baseline (clones 770 vs. 19(ST)A, FIGS. 31A and 31B). Mass spectrometry analysis confirmed that sialic acid content was increased in 19(ST)A (FIG. 31C).

TABLE 9
Pharmacokinetic effects of increased and α-2,6-siaylation

| | | | | | Half- | |
|---|---|---|---|---|---|---|
| | | | | | Life | |
| Construct | SignalSequence | CatalyticDomain | NucleaseDomain | Fc Domain | (Hours) | AUC |
| 770 | | | | | 37 | 3381 |
| 1 | | | E592N | | 40 | 1935 |
| 2 | | K369N/I371T | | | 35 | 2561 |
| 2(ST) | | K369N/I371T | | | 36 | 4426 |
| 9 | V29N | | S766N | H1064KN1065F | 65 | 6047 |
| 10 | C25NK27T | | S766N | H1064K N1065F | 55 | 7735 |
| 1(ST) | | | E592N | | 49 | 8379 |
| 15(ST) | V29N | | E592N | H1064K N1065F | 88 | 13871 |
| 13 | V29N | | E592N | M883Y | 70 | 14978 |
| | | | | S885TT887E | | |
| 9(ST) | V29N | | S766N | H1064KN1065F | 86 | 15099 |
| 18 | | | E592N | M883Y | 83 | 19638 |
| | | | | S885TT887E | | |
| 14 | | | | M883Y | 96 | 20360 |
| | | | | S885TT887E | | |
| 10(ST) | C25NK27T | | S766N | H1064K N1065F | 70.1 | 18207 |
| 15 | V29N | | E592N | H1064K N1065F | 99 | 22620 |
| 14(ST) | | | | M883Y | 119 | 22793 |
| | | | | S885TT887E | | |
| 9(ST)A | V29N | | S766N | H1064K N1065F | 115 | 26312 |
| 18(ST) | | | E592N | M883Y | 97.5 | 14263 |
| | | | | S885TT887E | | |
| 7 | | I256T | | | 67 | 26598 |
| 17 | V29N | I256T | P534N, V536T | M883Y | 120 | 33752 |
| | | | | S885TT887E | | |
| 19(ST) | | I256T | | M883Y | 170 | 35252 |
| | | | | S885TT887E | | |
| 17(ST) | V29N | I256T | P534N, V536T | M883YS885TT887E | 205 | 36595 |
| 19(ST)A | | I256T | | M883Y | 204 | 44742 |
| | | | | S885TT887E | | |

### In Vivo Confirmation of the Disease-Reversing Ability of Bio Molecularly Engineered ENPP1-Fc

The results presented above demonstrate that by evaluating a modestly-sized panel of glycosylation- and protein-engineered variants of ENPP1-Fc and by further producing these nascent therapeutics using a novel biomanufacturing platform that ensures a high level of “humanized” sialylation, specific candidates with substantially improved PK properties were identified. It was confirmed that the newly engineered forms of ENPP1-Fc retained disease-reversing activity in vivo by monitoring PPi in plasma, which is a biomarker for clinical efficacy of ENPP1 enzyme replacement therapy (16). To compare the pharmacodynamic effects of the above alterations, Enpp1^{asj/asj} mice were dosed with a single subcutaneous dose of 0.3 mg/Kg of 770 and 19 (ST) and measured plasma PPi and enzyme presence in plasma for 11 days (FIG. 31D). Plasma PPi in mice dosed with parent ENPP1-Fc (clone 770) required a weekly dose of 7.5 mg/Kg to maintain plasma PPi in the normal range (FIG. 31E), whereas a single dose of 19 (ST) elevated plasma PPi at or above the normal range for approximately 250 hours (FIG. 31D), representing a PK gain of ˜37-fold. FIG. 31D also demonstrates that plasma PPi was more variable than plasma enzyme concentration, providing evidence that the pharmacodynamic response has greater variability than enzyme clearance.

### Discussion

In this study, complementary strategies were sequentially applied to optimize the pharmacologic and pharmacodynamic properties of a therapeutic enzyme designed for the treatment of ENPP1 deficiency while fully maintaining catalytic activity. This approach is novel in two ways. First, in the past the various strategies used (e.g., Fc fusion proteins and mutations, building in N-glycosylation sites, and using downstream m biomanufacturing strategies to increase sialylation) have been evaluated individually but have not been combined to collectively gain the aggregate benefit of each effect. Second, although certain of these methods have been used to optimize monoclonal antibodies or hormone therapeutics, they have not been applied to enzyme therapeutics. It was demonstrated that by sequentially combining these various orthogonal and complementary strategies, pharmacokinetic responses could be additively improved and enhance the in vivo efficacy of a subcutaneously dosed enzyme biologic.

The single beneficial N-GCS site we identified, however, provided substantially greater improvement than any of other technique employed singly. The beneficial N-GCS was identified in a rational manner by referencing isoenzymes and other ENPP family members, and by avoiding areas of ENPP1 where inactivating mutations have been reported. These strategies allowed for prioritizing several dozen N-GCS sites for high throughput screening, which identified potentially favorable N-GCS sites in every ENPP1 protein domain designed to install N-glycans that would shield a majority of the ENPP1 protein surface. It was found that the introduction of N-GCS reduced either the protein expression or the catalytic activity of most of these ENPP1 glycoforms with the substantial benefit only observed in one instance, which was the I256T mutation in clone 7. Interestingly, this N-GCS was an outlier in that it was not designed to cover the surface of the ENPP1 protein but instead occurs in the insertion loop near the catalytic residue responsible for the nucleophilic attack of the catalyst on the substrate; this unusually positioned glycan was included in our screening strategy because of homology with the ENPP3 family member. The introduction of this site into ENPP1 increased the bioavailability by approximately ˜8-fold, primarily by increasing Cmax after subcutaneous dosing, presumably by enhancing the absorbance of the subcutaneous bolus into the blood (FIG. 30B).

The second strategy, was based on protein engineering, we optimized FcRN recycling of the Fc domain; in contrast to the glycoengineering strategy, this method enhanced PK by increasing the half-life with little effect on Cmax. Quantitatively comparing the two techniques, adding a glycan at position 254 had a greater impact than Fc optimization, increasing AUC by ˜8-fold compared with a ˜6-fold increase from MST Fc mutations (clones 7 and 14 vs 770, FIG. 30A). Using both techniques in combination further increased AUC to 10-fold above the parent 770, or an additional 2-fold increase.

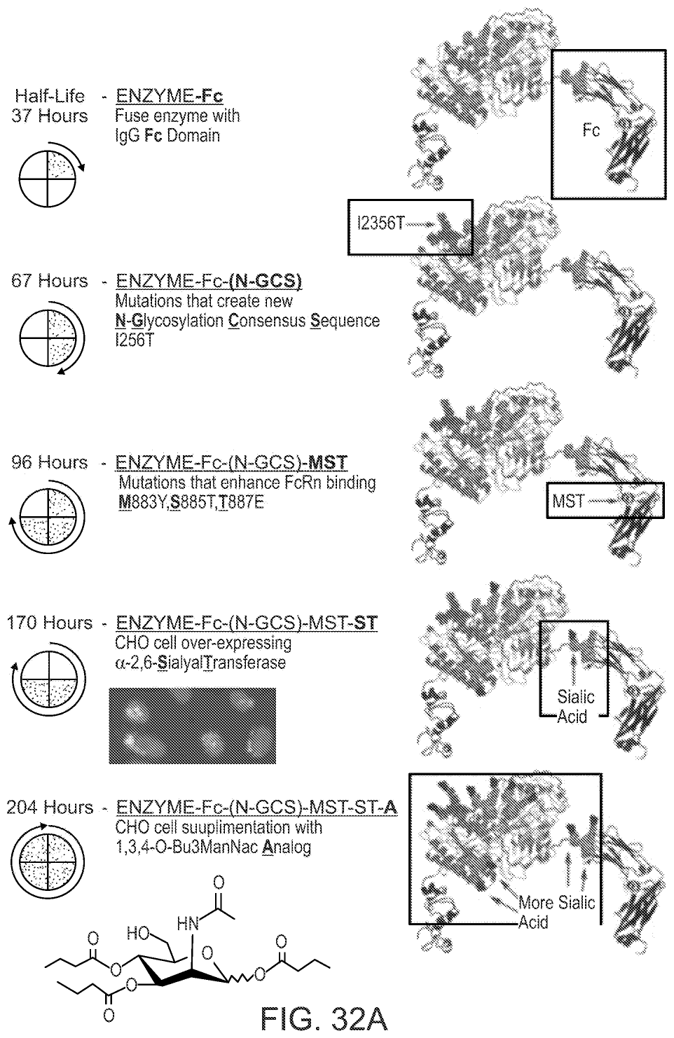

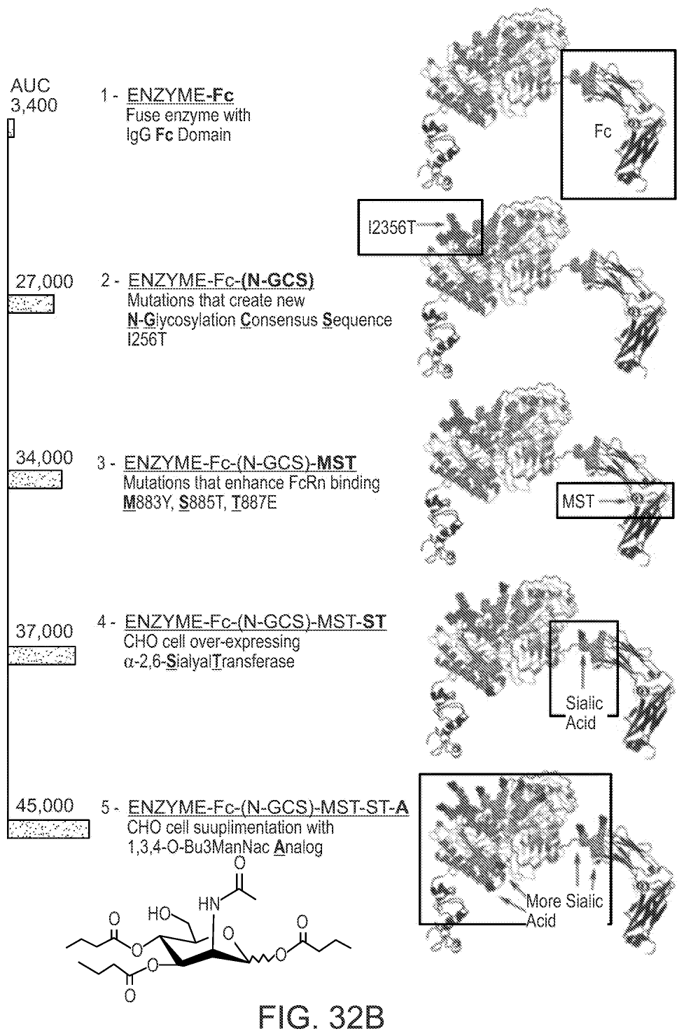

Glyco- and protein engineering represented by clone 17, moreover usually applied separately, constitute the current limits of “upstream” efforts to improve therapeutic proteins. In this study it was reasoned that additional benefits could be obtained by implementing downstream biomanufacturing advances. This premise was demonstrated by expressing ENPP1-Fc with combined 1256T and the MST mutations in α-2,6-ST over-expressing CHO cells and growing the cultures in the sialic acid precursor 1,3,4-O-Bu₃ManNAc; these two steps increased the AUC an additional 3 fold, resulting in a biologic with a ˜13-fold increase over the parent biologic (Clone 19(ST) A vs 770, FIG. 31B), demonstrating the importance of glyco-polishing to the pharmacokinetic properties of the biologic. The pharmacodynamic (PD) effect of these changes was substantial; the increase in potency of the optimized therapeutic, as judged from enhancement of PD, was ˜37 fold (i.e., whereas construct 770 required 7.5 mg/Kg to normalize plasma PPi for 7 days, 0.3 mg/Kg of construct 19(ST) was able to normalize plasma PPi for 10.4 days). The overall strategy with a step by step illustration in PK improvements is provided in FIG. 32.

The results obtained herein, provide for a bi-monthly or monthly dosing schedule of best ENPP1-Fc construct, a dosing scheme highly favorable for the chronic therapy required for life-threatening and debilitating diseases of ENPP1 deficiency such as GACI and ARHR2, and for diseases of vascular and soft tissue calcification induced by low PPi such as Pseudoxanthoma Elasticum (PXE) and Chronic Kidney Disease Bone Mineralization Disorder (CKD-MBD).

### REFERENCES

1. van Meeteren L A, Ruurs P, Christodoulou E, Goding J W, Takakusa H, Kikuchi K, Perrakis A, Nagano T, Moolenaar W H. Inhibition of autotaxin by lysophosphatidic acid and sphingosine 1-phosphate. J Biol Chem. 2005; 280(22):21155-61. PubMed PMID: 15769751.
2. Tanaka M, Okudaira S, Kishi Y, Ohkawa R, Iseki S, Ota M, Noji S, Yatomi Y, Aoki J, Arai H. Autotaxin stabilizes blood vessels and is required for embryonic vasculature by producing lysophosphatidic acid. J Biol Chem. 2006; 281(35):25822-30. PubMed PMID: 16829511.
3. Nam S W, Clair T, Kim Y S, McMarlin A, Schiffmann E, Liotta L A, Stracke M L. Autotaxin (NPP-2), a metastasis-enhancing motogen, is an angiogenic factor. Cancer Res. 2001; 61(18):6938-44. PubMed PMID: 11559573.
4. Manning T J, Jr., Rosenfeld S S, Sontheimer H. Lysophosphatidic acid stimulates actomyosin contraction in astrocytes. J Neurosci Res. 1998; 53(3):343-52. PubMed PMID: 9698162.
5. Umezu-Goto M, Kishi Y, Taira A, Hama K, Dohmae N, Takio K, Yamori T, Mills G B, Inoue K, Aoki J, Arai H. Autotaxin has lysophospholipase D activity leading to tumor cell growth and motility by lysophosphatidic acid production. J Cell Biol. 2002; 158(2):227-33. PubMed PMID: 12119361.
6. Nam S W, Clair T, Campo C K, Lee H Y, Liotta L A, Stracke M L. Autotaxin (ATX), a potent tumor motogen, augments invasive and metastatic potential of ras-transformed cells. Oncogene. 2000; 19(2):241-7. PubMed PMID: 10645002.
7. Hoelzinger D B, Nakada M, Demuth T, Rosensteel T, Reavie L B, Berens M E. Autotaxin: a secreted autocrine/paracrine factor that promotes glioma invasion. J Neurooncol. 2008; 86(3):297-309. PubMed PMID: 17928955.
8. Saunders L P, Ouellette A, Bandle R, Chang W C, Zhou H, Misra R N, De La Cruz E M, Braddock D T. Identification of small-molecule inhibitors of autotaxin that inhibit melanoma cell migration and invasion. Mol Cancer Ther. 2008; 7(10):3352-62. PubMed PMID: 18852138.
9. Liu S, Umezu-Goto M, Murph M, Lu Y, Liu W, Zhang F, Yu S, Stephens L C, Cui X, Murrow G, Coombes K, Muller W, Hung M C, Perou C M, Lee A V, Fang X, Mills G B. Expression of autotaxin and lysophosphatidic acid receptors increases mammary tumorigenesis, invasion, and metastases. Cancer Cell. 2009; 15(6):539-50. PubMed PMID: 19477432.
10. Kishi Y, Okudaira S, Tanaka M, Hama K, Shida D, Kitayama J, Yamori T, Aoki J, Fujimaki T, Arai H. Autotaxin is overexpressed in glioblastoma multiforme and contributes to cell motility of glioblastoma by converting lysophosphatidylcholine to lysophosphatidic acid. J Biol Chem. 2006; 281(25): 17492-500. PubMed PMID: 16627485.
11. Okawa A, Nakamura I, Goto S, Moriya H, Nakamura Y, Ikegawa S. Mutation in Npps in a mouse model of ossification of the posterior longitudinal ligament of the spine. Nature genetics. 1998; 19(3):271-3. Epub 1998/07/14. doi: 10.1038/956. PubMed PMID: 9662402.
12. Nakamura I, Ikegawa S, Okawa A, Okuda S, Koshizuka Y, Kawaguchi H, Nakamura K, Koyama T, Goto S, Toguchida J, Matsushita M, Ochi T, Takaoka K, Nakamura Y. Association of the human NPPS gene with ossification of the posterior longitudinal ligament of the spine (OPLL). Human genetics. 1999; 104(6):492-7. Epub 1999/08/24. PubMed PMID: 10453738.
13. Levy-Litan V, Hershkovitz E, Avizov L, Leventhal N, Bercovich D, Chalifa-Caspi V, Manor E, Buriakovsky S, Hadad Y, Goding J, Parvari R. Autosomal-recessive hypophosphatemic rickets is associated with an inactivation mutation in the ENPP1 gene. American journal of human genetics. 2010; 86(2):273-8. Epub 2010/02/09. doi: 10.1016/j.ajhg.2010.01.010. PubMed PMID: 20137772; PMCID: 2820183.
14. Lorenz-Depiereux B, Schnabel D, Tiosano D, Hausler G, Strom T M. Loss-of-function ENPP1 mutations cause both generalized arterial calcification of infancy and autosomal-recessive hypophosphatemic rickets. American journal of human genetics. 2010; 86(2):267-72. doi: 10.1016/j.ajhg.2010.01.006. PubMed PMID: 20137773; PMCID: PMC2820166.
15. Rutsch F, Ruf N, Vaingankar S, Toliat M R, Suk A, Hohne W, Schauer G, Lehmann M, Roscioli T, Schnabel D, Epplen J T, Knisely A, Superti-Furga A, McGill J, Filippone M, Sinaiko A R, Vallance H, Hinrichs B, Smith W, Ferre M, Terkeltaub R, Nurnberg P. Mutations in ENPP1 are associated with ‘idiopathic’ infantile arterial calcification. Nature genetics. 2003; 34(4):379-81. Epub 2003/07/26. doi: 10.1038/ng1221. PubMed PMID: 12881724.
16. Rutsch F, Vaingankar S, Johnson K, Goldfine I, Maddux B, Schauerte P, Kalhoff H, Sano K, Boisvert W A, Superti-Furga A, Terkeltaub R. PC-1 nucleoside triphosphate pyrophosphohydrolase deficiency in idiopathic infantile arterial calcification. The American journal of pathology. 2001; 158(2):543-54. Epub 2001/02/13. doi: 10.1016/S0002-9440(10)63996-X. PubMed PMID: 11159191; PMCID: 1850320.
17. Li Q, Guo H, Chou D W, Berndt A, Sundberg J P, Uitto J. Mutant Enpplasj mice as a model for generalized arterial calcification of infancy. Disease models & mechanisms. 2013; 6(5): 1227-35. doi: 10.1242/dmm.012765. PubMed PMID: 23798568; PMCID: 3759342.
18. Albright R A, Chang W C, Robert D, Ornstein D L, Cao W, Liu L, Redick M E, Young J I, De La Cruz E M, Braddock D T. NPP4 is a procoagulant enzyme on the surface of vascular endothelium. Blood. 2012; 120(22):4432-40. Epub 2012/09/22. doi: 10.1182/blood-2012-04-425215. PubMed PMID: 22995898; PMCID: PMC4017314.
19. Zalatan J G, Fenn T D, Brunger A T, Herschlag D. Structural and functional comparisons of nucleotide pyrophosphatase/phosphodiesterase and alkaline phosphatase: implications for mechanism and evolution. Biochemistry. 2006; 45(32):9788-803. PubMed PMID: 16893180.
20. O'Neill W C, Sigrist M K, McIntyre C W. Plasma pyrophosphate and vascular calcification in chronic kidney disease. Nephrol Dial Transplant. 2010; 25(1): 187-91. doi: 10.1093/ndt/gfp362. PubMed PMID: 19633093; PMCID: 4326300.
21. Oheim R, Zimmerman K, Maulding N D, Stürznickel J, v. Kroge S, Kavanagh D, Stabach P, Kornak U, Tommasini S M, Horowitz M, Amling M, Thompson D, Schinke T, Busse B, Carpenter T O, Braddock D T. Human heterozygous ENPP1 deficiency is associated with early onset osteoporosis, a phenotype recapitulated in a mouse model of Enpp1 deficiency. Journal of Bone and Mineral Research. 2019. Epub November 5th. doi: 10.1002/jbmr.3911.
22. Meyer J L. Can biological calcification occur in the presence of pyrophosphate? Arch Biochem Biophys. 1984; 231(1): 1-8. PubMed PMID: 6326671.
23. Bryant J, White W. A case of calcification of the arteries and obliterative endarteritis, associated with hydronephrosis, in a child aged six months. Guy's Hospital Reports. 1901; 55:17-28.
24. Albright R A, Stabach P, Cao W, Kavanagh D, Mullen I, Braddock A A, Covo M S, Tehan M, Yang G, Cheng Z, Bouchard K, Yu Z X, Thorn S, Wang X, Folta-Stogniew E J, Negrete A, Sinusas A J, Shiloach J, Zubal G, Madri J A, De La Cruz E M, Braddock D T. ENPP1-Fc prevents mortality and vascular calcifications in rodent model of generalized arterial calcification of infancy. Nat Commun. 2015; 6:10006. Epub 2015/12/02. doi: 10.1038/ncomms10006. PubMed PMID: 26624227; PMCID: PMC4686714.
25. Nitschke Y, Yan Y, Buers I, Kintziger K, Askew K, Rutsch F. ENPP1-Fc prevents neointima formation in generalized arterial calcification of infancy through the generation of AMP. Exp Mol Med. 2018; 50(10): 139. Epub 2018/10/30. doi: 10.1038/s12276-018-0163-5. PubMed PMID: 30369595; PMCID: PMC6204430.
26. Rutsch F, Boyer P, Nitschke Y, Ruf N, Lorenz-Depierieux B, Wittkampf T, Weissen-Plenz G, Fischer R J, Mughal Z, Gregory J W, Davies J H, Loirat C, Strom T M, Schnabel D, Nurnberg P, Terkeltaub R, Group G S. Hypophosphatemia, hyperphosphaturia, and bisphosphonate treatment are associated with survival beyond infancy in generalized arterial calcification of infancy. Circulation Cardiovascular genetics. 2008; 1(2):133-40. doi: 10.1161/CIRCGENETICS.108.797704. PubMed PMID: 20016754; PMCID: 2794045.
27. Sholler G F, Yu J S, Bale P M, Hawker R E, Celermajer J M, Kozlowski K. Generalized arterial calcification of infancy: three case reports, including spontaneous regression with long-term survival. The Journal of pediatrics. 1984; 105(2):257-60. Epub 1984/08/01. PubMed PMID: 6747757.
28. Brachet C, Mansbach A L, Clerckx A, Deltenre P, Heinrichs C. Hearing loss is part of the clinical picture of ENPP1 loss of function mutation. Horm Res Paediatr. 2014; 81(1):63-6. Epub 2013/11/13. doi: 10.1159/000354661. PubMed PMID: 24216977.
29. Steichen-Gersdorf E, Lorenz-Depiereux B, Strom T M, Shaw N J. Early onset hearing loss in autosomal recessive hypophosphatemic rickets caused by loss of function mutation in ENPP1. J Pediatr Endocrinol Metab. 2015; 28(7-8):967-70. Epub 2015/03/06. doi: 10.1515/jpem-2014-0531. PubMed PMID: 25741938.
30. Kotwal A, Ferrer A, Kumar R, Singh R J, Murthy V, Schultz-Rogers L, Zimmermann M, Lanpher B, Zimmerman K, Stabach P R, Klee E, Braddock D T, Wermers R A. Clinical and Biochemical Phenotypes in a Family with ENPP1 Mutations. Journal of bone and mineral research: the official journal of the American Society for Bone and Mineral Research. 2019. Epub 2019/12/12. doi: 10.1002/jbmr.3938. PubMed PMID: 31826312.
31. Marrott P K, Newcombe K_{D}, Becroft D M, Friedlander D H. Idiopathic infantile arterial calcification with survival to adult life. Pediatr Cardiol. 1984; 5(2):119-22. doi: 10.1007/BF02424963. PubMed PMID: 6473121.
32. Guan X, Chaffey P K, Wei X, Gulbranson D R, Ruan Y, Wang X, Li Y, Ouyang Y, Chen L, Zeng C, Koelsch T N, Tran A H, Liang W, Shen J, Tan Z. Chemically Precise Glycoengineering Improves Human Insulin. ACS Chem Biol. 2018; 13(1):73-81. Epub 2017/11/02. doi: 10.1021/acschembio.7b00794. PubMed PMID: 29090903; PMCID: PMC6287623.
33. Egrie J C, Dwyer E, Browne J K, Hitz A, Lykos M A. Darbepoetin alfa has a longer circulating half-life and greater in vivo potency than recombinant human erythropoietin. Exp Hematol. 2003; 31(4):290-9. Epub 2003/04/15. doi: 10.1016/s0301-472x(03)00006-7. PubMed PMID: 12691916.
34. Kato K, Nishimasu H, Oikawa D, Hirano S, Hirano H, Kasuya G, Ishitani R, Tokunaga F, Nureki O. Structural insights into cGAMP degradation by Ecto-nucleotide pyrophosphatase phosphodiesterase 1. Nat Commun. 2018; 9(1):4424. Epub 2018/10/26. doi: 10.1038/s41467-018-06922-7. PubMed PMID: 30356045; PMCID: PMC6200793.
35. Dall'Acqua W F, Woods R M, Ward E S, Palaszynski S R, Patel N K, Brewah Y A, Wu H, Kiener P A, Langermann S. Increasing the affinity of a human IgG1 for the neonatal Fc receptor: biological consequences. J Immunol. 2002; 169(9):5171-80. Epub 2002/10/23. PubMed PMID: 12391234.
36. Chung C H, Mirakhur B, Chan E, Le Q T, Berlin J, Morse M, Murphy B A, Satinover S M, Hosen J, Mauro D, Slebos R J, Zhou Q, Gold D, Hatley T, Hicklin D J, Platts-Mills T A. Cetuximab-induced anaphylaxis and IgE specific for galactose-alpha-1,3-galactose. N Engl J Med. 2008; 358(11): 1109-17. Epub 2008/03/14. doi: 10.1056/NEJMoa074943. PubMed PMID: 18337601; PMCID: PMC2361129.
37. Buettner M J, Shah S R, Saeui C T, Ariss R, Yarema K J. Improving Immunotherapy Through Glycodesign. Front Immunol. 2018; 9:2485. Epub 2018/11/20. doi: 10.3389/fimmu.2018.02485. PubMed PMID: 30450094; PMCID: PMC6224361.
38. Aich U, Campbell C T, Elmouelhi N, Weier C A, Sampathkumar S G, Choi S S, Yarema K J. Regioisomeric SCFA attachment to hexosamines separates metabolic flux from cytotoxicity and MUCI suppression. ACS Chem Biol. 2008; 3(4):230-40. Epub 2008/03/15. doi: 10.1021/cb7002708. PubMed PMID: 18338853.
39. Almaraz R T, Aich U, Khanna H S, Tan E, Bhattacharya R, Shah S, Yarema K J. Metabolic oligosaccharide engineering with N-Acyl functionalized ManNAc analogs: cytotoxicity, metabolic flux, and glycan-display considerations. Biotechnol Bioeng. 2012; 109(4): 992-1006. Epub 2011/11/10. doi: 10.1002/bit.24363. PubMed PMID: 22068462; PMCID: PMC3288793.
40. Sinha S D, Bandi V K, Bheemareddy B R, Thakur P, Chary S, Mehta K, Pinnamareddy V R, Pandey R, Sreepada S, Durugkar S. Efficacy, tolerability and safety of darbepoetin alfa injection for the treatment of anemia associated with chronic kidney disease (CKD) undergoing dialysis: a randomized, phase-III trial. BMC Nephrol. 2019; 20(1):90. Epub 2019/03/15. doi: 10.1186/s12882-019-1209-1. PubMed PMID: 30866856; PMCID: PMC6417108.
41. Song R, Oren D A, Franco D, Seaman M S, Ho D D. Strategic addition of an N-linked glycan to a monoclonal antibody improves its HIV-1-neutralizing activity. Nat Biotechnol. 2013; 31(11): 1047-52. Epub 2013/10/08. doi: 10.1038/nbt.2677. PubMed PMID: 24097413; PMCID: PMC3825789.
42. Weng Y, Ishino T, Sievers A, Talukdar S, Chabot J R, Tam A, Duan W, Kerns K, Sousa E, He T, Logan A, Lee D, Li D, Zhou Y, Bernardo B, Joyce A, Kavosi M, O'Hara D M, Clark T, Guo J, Giragossian C, Stahl M, Calle R A, Kriz R, Somers W, Lin L. Glyco-engineered Long Acting FGF21 Variant with Optimal Pharmaceutical and Pharmacokinetic Properties to Enable Weekly to Twice Monthly Subcutaneous Dosing. Sci Rep. 2018; 8(1):4241. Epub 2018/03/11. doi: 10.1038/s41598-018-22456-w. PubMed PMID: 29523796; PMCID: PMC5844872.
43. Kallberg M, Wang H, Wang S, Peng J, Wang Z, Lu H, Xu J. Template-based protein structure modeling using the RaptorX web server. Nat Protoc. 2012; 7(8): 1511-22. Epub 2012/07/21. doi: 10.1038/nprot.2012.085. PubMed PMID: 22814390; PMCID: PMC4730388.
44. Yin B, Wang Q, Chung C Y, Bhattacharya R, Ren X, Tang J, Yarema K J, Betenbaugh M J. A novel sugar analog enhances sialic acid production and biotherapeutic sialylation in CHO cells. Biotechnology and Bioengineering. 2017; 114(8): 1899-902. doi: doi: 10.1002/bit.26291.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of producing modified host cells for producing engineered proteins or peptides comprising: transfecting host cells with an expression vector encoding a protein or peptide comprising one or more genetic mutations, wherein the one or more genetic mutations encode for one or more N-glycans; thereby, producing a modified protein peptide.

2. The method of claim 1, wherein the host cell comprises one or more enzymes that modulate N-glycan branching, sialylation or combinations thereof.

3. The method of claim 2, wherein the one or more enzymes that modulate N-glycan branching comprise N-acetylglucosaminyltransferases.

4. The method of claim 2, wherein the one or more enzymes that modulate sialylation comprise sialyltransferases.

5. The method of claim 2, wherein the one or more proteins that modulate sialylation comprise Golgi-Neu5Ac transporters.

6. The method of claim 2, wherein the one or more enzymes that modulate sialylation comprise sialic acid 9-phosphate synthase (SAS) or other bottlenecks in a sialic acid biosynthetic pathway.

7. The method of claim 2, wherein the host cell does not produce or encode one or more enzymes that modulate sialic acid metabolic flux.

8. The method of claim 7, wherein the host cell nucleic acid sequences lacks one or more enzymes that modulate sialic acid metabolic flux.

9. The method of claim 7, wherein the one or more enzymes that modulate sialic acid metabolic flux comprise GNE (UDP-N-acetylglucosamine 2-epimerase/N-acetylmannosamine kinase), RENBP (Renin-binding protein; N-GlcNAc 2-epimerase), NPL (N-acetylneuraminic acid lyase), NEU1 (Neurimindase 1), NEU2 (Neurimindase 2), NEU3 (Neurimindase 3), NEU4 (Neurimindase 4), Klotho or combinations thereof.

10. The method of claim 1 further comprising contacting the host cell with one or more hexosamine analogs.

11. The method of claim 10, wherein the hexosamine analog comprises high flux ManNAc analogs.

12. The method of claim 10, wherein the hexosamine analog comprises ManNAc analogs appended with N-acyl chemical functional groups comprising a hydrogen, an oxygen, a hydroxyl group, a halide, an amide, a methyl, —C(O)alkyl, —C(O)(CH₂)nCH₃, —(CH₂)₂CH₃, —(CH₂)₃CH₃, —(CH₂)₄CH₃, —CH(CH₃)₂ or —CH₂CH(CH₃)₂, unsaturated alkyl or a saturated alkyl.

13. The method of claim 10, wherein the hexosamine analog comprises GlcNAc analogs appended with N-acyl chemical functions groups comprising a hydrogen, an oxygen, a hydroxyl group, a halide, an amide, a methyl, —C(O)alkyl, —C(O)(CH₂)nCH₃, —(CH₂)₂CH₃, —(CH₂)₃CH₃, —(CH₂)₄CH₃, —CH(CH₃)₂ or —CH₂CH(CH₃)₂, unsaturated alkyl or a saturated alkyl.

14. The method of claim 10, wherein the hexosamine analog comprises GalNAc analogs appended with N-acyl chemical functional groups comprising a hydrogen, an oxygen, a hydroxyl group, a halide, an amide, a methyl, —C(O)alkyl, —C(O)(CH₂)nCH₃, —(CH₂)₂CH₃, —(CH₂)₃CH₃, —(CH₂)₄CH₃, —CH(CH₃)₂ or —CH₂CH(CH₃)₂, unsaturated alkyl or a saturated alkyl.

15. The method of claim 10, wherein the hexosamine analog is a fucose analog appended with N-acyl chemical functional groups comprising a hydrogen, an oxygen, a hydroxyl group, a halide, an amide, a methyl, —C(O)alkyl, —C(O)(CH₂)nCH₃, —(CH₂)₂CH₃, —(CH₂)₃CH₃, —(CH₂)₄CH₃, —CH(CH₃)₂ or —CH₂CH(CH₃)₂, unsaturated alkyl or a saturated alkyl.

16-27. (canceled)

28. A modified host cell comprising one or more nucleic acid sequences encoding one or more enzymes that modulate N-glycan branching, sialylation or combinations thereof.

29-36. (canceled)

37. An engineered protein comprising one or more modified amino acid sequences, wherein the modified amino acid sequences comprise an N-glycan consensus sequence.

38-47. (canceled)

48. A modified immunoglobulin comprising one or more N-glycan groups.

49. The modified immunoglobulin of claim 48, wherein the immunoglobulin comprises at least one amino acid sequence comprising SEQ ID NO: 1-12.

50. A method of treating cancer comprising administering to a subject in need thereof, a pharmaceutical composition comprising a therapeutically effective amount of the engineered protein of claim 37.

51-54. (canceled)


SEQUENCE LISTING (60 sequences)

SEQ ID NO: 1 - protein, 465 aa, Artificial Sequence
  Description of Artificial Sequence Synthetic polypeptide
METDTLLLWVLLLWVPGSTGDEVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRTPYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 237 aa, Artificial Sequence
  Description of Artificial Sequence Synthetic polypeptide
MRVPAQLLGLLLLWLPGARCAGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 465 aa, Artificial Sequence
  Description of Artificial Sequence Synthetic polypeptide
METDTLLLWVLLLWVPGSTGDEVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRTPYWGNGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 237 aa, Artificial Sequence
  Description of Artificial Sequence Synthetic polypeptide
MRVPAQLLGLLLLWLPGARCAGSNITVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 472 aa, Artificial Sequence
  Description of Artificial Sequence Synthetic polypeptide
METDTLLLWVLLLWVPGSTGDQLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARTPTVTGDWFDPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 238 aa, Artificial Sequence
  Description of Artificial Sequence Synthetic polypeptide
MRVPAQLLGLLLLWLPGARCGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 472 aa, Artificial Sequence
  Description of Artificial Sequence Synthetic polypeptide
METDTLLLWVLLLWVPGSTGDQLQLQESGPGLVKPNQTLSLTCTVSGGSISSGGYYWSWIRQHPGKGLEWIGYIYYSGSTYYNPSLKSRVTISVDTSKNQFSLKLSSVTAADTAVYYCARTPTVTGDWFDPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 472 aa, Artificial Sequence
  Description of Artificial Sequence Synthetic polypeptide
METDTLLLWVLLLWVPGSTGDQLQLQESGPGLVKPSQTLSLTCTVSGGSISSGGYYWSWIRQHPGKGLEWIGYIYYSGSTYYNPSLKSRVTISVDTSKNQFSLKLSNVTAADTAVYYCARTPTVTGDWFDPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 472 aa, Artificial Sequence
  Description of Artificial Sequence Synthetic polypeptide
METDTLLLWVLLLWVPGSTGDQLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARTPTVTGDWFDPWGNGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, Artificial Sequence
  Description of Artificial Sequence Synthetic polypeptide
MRVPAQLLGLLLLWLPGARCGSNFMLTQPHSVSESPGKTVTISCTRSSGSIASNYVQWYQQRPGSSPTTVIYEDNQRPSGVPDRFSGSINSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 238 aa, Artificial Sequence
  Description of Artificial Sequence Synthetic polypeptide
MRVPAQLLGLLLLWLPGARCGSNFMLTQPHSVSESPGKTVTISCTRSSGSIASNYVQWYQQRPGSSPTTVIYEDNQRPSGVPDRFSGSIDNSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 238 aa, Artificial Sequence
  Description of Artificial Sequence Synthetic polypeptide
MRVPAQLLGLLLLWLPGARCGSNFMLTQPHSVSESPGKTVTISCTRSSGSIASNYVQWYQQRPGSSPTTVIYEDNQRPSGVPDRFSGSIDSSSNSASLTISGLKTNDTADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 22 aa, Artificial Sequence
  Description of Artificial Sequence Synthetic peptide
SGTFFWPGSDVEINGTFPDIYK

SEQ ID NO: 14 - DNA, 23 bp, Artificial Sequence
  Description of Artificial Sequence Synthetic primer
gagggcctatttcccatgattcc

SEQ ID NO: 15 - DNA, 24 bp, Artificial Sequence
  Description of Artificial Sequence Synthetic oligonucleotide
caccgctacacacgatcgttagag

SEQ ID NO: 16 - DNA, 24 bp, Artificial Sequence
  Description of Artificial Sequence Synthetic oligonucleotide
caccgttcgagtgatgcggaagaa

SEQ ID NO: 17 - DNA, 24 bp, Artificial Sequence
  Description of Artificial Sequence Synthetic oligonucleotide
cacccggcatctagaagctcagcg

SEQ ID NO: 18 - DNA, 24 bp, Artificial Sequence
  Description of Artificial Sequence Synthetic oligonucleotide
cacctgtgctagagaatgtatcag

SEQ ID NO: 19 - DNA, 24 bp, Artificial Sequence
  Description of Artificial Sequence Synthetic oligonucleotide
aaacctctaacgatcgtgtgtagc

SEQ ID NO: 20 - DNA, 24 bp, Artificial Sequence
  Description of Artificial Sequence Synthetic oligonucleotide
aaacttcttccgcatcactcgaac

SEQ ID NO: 21 - DNA, 24 bp, Artificial Sequence
  Description of Artificial Sequence Synthetic oligonucleotide
aaaccgctgagcttctagatgccg

SEQ ID NO: 22 - DNA, 24 bp, Artificial Sequence
  Description of Artificial Sequence Synthetic oligonucleotide
aaacctgatacattctctagcaca

SEQ ID NO: 23 - DNA, 23 bp, Artificial Sequence
  Description of Artificial Sequence Synthetic primer
gtaacagtgggaatcaggtagaa

SEQ ID NO: 24 - DNA, 20 bp, Artificial Sequence
  Description of Artificial Sequence Synthetic primer
agcaagagccgtgaagtaaa

SEQ ID NO: 25 - DNA, 22 bp, Artificial Sequence
  Description of Artificial Sequence Synthetic primer
actgtggtatcctaccctatcc

SEQ ID NO: 26 - DNA, 20 bp, Artificial Sequence
  Description of Artificial Sequence Synthetic primer
cgaactccacagctgctatt

SEQ ID NO: 27 - DNA, 22 bp, Artificial Sequence
  Description of Artificial Sequence Synthetic primer
cactcccatgaccaggaatatg

SEQ ID NO: 28 - DNA, 22 bp, Artificial Sequence
  Description of Artificial Sequence Synthetic primer
ggtgagggcttggaatatactg

SEQ ID NO: 29 - DNA, 24 bp, Artificial Sequence
  Description of Artificial Sequence Synthetic oligonucleotide
caccgagcagcagtggaatccggt

SEQ ID NO: 30 - DNA, 24 bp, Artificial Sequence
  Description of Artificial Sequence Synthetic oligonucleotide
aaacaccggattccactgctgctc

SEQ ID NO: 31 - DNA, 24 bp, Artificial Sequence
  Description of Artificial Sequence Synthetic oligonucleotide
caccgcgtgagaaatagtaggcgt

SEQ ID NO: 32 - DNA, 24 bp, Artificial Sequence
  Description of Artificial Sequence Synthetic oligonucleotide
aaacacgcctactatttctcacgc

SEQ ID NO: 33 - DNA, 19 bp, Artificial Sequence
  Description of Artificial Sequence Synthetic primer
tatcagcctgctccctcaa

SEQ ID NO: 34 - DNA, 22 bp, Artificial Sequence
  Description of Artificial Sequence Synthetic primer
ctgatgacctgggtttgatctc

SEQ ID NO: 35 - DNA, 18 bp, Artificial Sequence
  Description of Artificial Sequence Synthetic primer
agccgtgtgttcctgttt

SEQ ID NO: 36 - DNA, 21 bp, Artificial Sequence
  Description of Artificial Sequence Synthetic primer
gagtagtgggagcagctttac

SEQ ID NO: 37 - DNA, 23 bp, Artificial Sequence
  Description of Artificial Sequence Synthetic oligonucleotide
cctaccggattccactgctgctc

SEQ ID NO: 38 - DNA, 20 bp, Artificial Sequence
  Description of Artificial Sequence Synthetic oligonucleotide
cctacgggttaatggggagc

SEQ ID NO: 39 - DNA, 23 bp, Artificial Sequence
  Description of Artificial Sequence Synthetic oligonucleotide
cctaccggattccagcgctgctc

SEQ ID NO: 40 - DNA, 14 bp, Artificial Sequence
  Description of Artificial Sequence Synthetic oligonucleotide
tctaatgcgtgctc

SEQ ID NO: 41 - DNA, 23 bp, Artificial Sequence
  Description of Artificial Sequence Synthetic oligonucleotide
cctacgcctactatttctcacgc

SEQ ID NO: 42 - DNA, 21 bp, Artificial Sequence
  Description of Artificial Sequence Synthetic oligonucleotide
cctacgtactatttctcacgc

SEQ ID NO: 43 - DNA, 17 bp, Artificial Sequence
  Description of Artificial Sequence Synthetic oligonucleotide
cctactatttctcatgc

SEQ ID NO: 44 - DNA, 111 bp, Artificial Sequence
  Description of Artificial Sequence Synthetic polynucleotide
  modified_base (7)..(94): a, c, t, g, unknown or other
cctacgnnnnnnnnnnnnnnnnnnnnnnnnnnnnnnnnnnnnnnnnnnnnnnnnnnnnnnnnnnnnnnnnnnnnnnnnnnnnnnnnnnnnnnnncctactatttctcacgc

SEQ ID NO: 45 - DNA, 20 bp, Artificial Sequence
  Description of Artificial Sequence Synthetic oligonucleotide
cctacgactatttctcacgc

SEQ ID NO: 46 - DNA, 17 bp, Artificial Sequence
  Description of Artificial Sequence Synthetic oligonucleotide
cctactatttctcacgc

SEQ ID NO: 47 - DNA, 59 bp, Artificial Sequence
  Description of Artificial Sequence Synthetic oligonucleotide
gactttgacattagcaccaggctacacacgatcgtagaggggaagatgaagcagccatg

SEQ ID NO: 48 - DNA, 60 bp, Artificial Sequence
  Description of Artificial Sequence Synthetic oligonucleotide
acgatcgttagaggggaagatgaagcagccatggtagagtcagtaggcctagctctagtg

SEQ ID NO: 49 - DNA, 59 bp, Artificial Sequence
  Description of Artificial Sequence Synthetic oligonucleotide
acgatcgtagaggggaagatgaagcagccatggtagagtcagtaggcctagctctagtg

SEQ ID NO: 50 - DNA, 61 bp, Artificial Sequence
  Description of Artificial Sequence Synthetic oligonucleotide
tcacagggagcaaggagatggttcgagtggtgcggaaagaagggcattgagcatcatccca

SEQ ID NO: 51 - DNA, 59 bp, Artificial Sequence
  Description of Artificial Sequence Synthetic oligonucleotide
tcatttcacagggagcaaggagatggttcgagtgatgcggaagaagggcattgagcatc

SEQ ID NO: 52 - DNA, 60 bp, Artificial Sequence
  Description of Artificial Sequence Synthetic oligonucleotide
tcatttcacagggagcaaggagatggttcgagtgatgcggaaagaagggcattgagcatc

SEQ ID NO: 53 - DNA, 64 bp, Artificial Sequence
  Description of Artificial Sequence Synthetic oligonucleotide
ctcaattttgagcacttccgcctcgcttgagcttctagatgccgcaaaagcaggtaccctgttc

SEQ ID NO: 54 - DNA, 59 bp, Artificial Sequence
  Description of Artificial Sequence Synthetic oligonucleotide
cacaggtatggatgtattgtcgcttgtacctcaattttgagcacttccgcctcgctgag

SEQ ID NO: 55 - DNA, 60 bp, Artificial Sequence
  Description of Artificial Sequence Synthetic oligonucleotide
cacaggtatggatgtattgtcgcttgtacctcaattttgagcacttccgcctcgcttgag

SEQ ID NO: 56 - DNA, 60 bp, Artificial Sequence
  Description of Artificial Sequence Synthetic oligonucleotide
cacaggtatggatgtattgtcgcttgtacctcaattttgagcacttccgcctcgcctgag

SEQ ID NO: 57 - DNA, 61 bp, Artificial Sequence
  Description of Artificial Sequence Synthetic oligonucleotide
tggcagagggatggacaagttgtgctagagaatgtattcagaggatggcaaaagaactccc

SEQ ID NO: 58 - DNA, 59 bp, Artificial Sequence
  Description of Artificial Sequence Synthetic oligonucleotide
gagaatgtatcagaggatggcaaaagaactccctggttgccttggaagatgccagaacc

SEQ ID NO: 59 - DNA, 60 bp, Artificial Sequence
  Description of Artificial Sequence Synthetic oligonucleotide
gagaatgtattcagaggatggcaaaagaactccctggttgccttggaagatgccagaacc

SEQ ID NO: 60 - DNA, 60 bp, Artificial Sequence
  Description of Artificial Sequence Synthetic oligonucleotide
gagaatgtatccagaggatggcaaaagaactccctggttgccttggaagatgccagaacc